(19) United States
(12) Patent Application Publication
(10) Pub. No.: US 2023/0123856 A1
(43) Pub. Date: Apr. 20, 2023

(54) 5-FLUORONICOTINAMIDE DERIVATIVES AND USES THEREOF

(71) Applicant: Tenaya Therapeutics, Inc., South San Francisco, CA (US)

(72) Inventors: Mohammad A. MANDEGAR, South San Francisco, CA (US); Snahel PATEL, South San Francisco, CA (US); Ulhas BHATT, South San Francisco, CA (US); Pingyu DING, South San Francisco, CA (US); Martin HOLAN, South San Francisco, CA (US); John LEE, South San Francisco, CA (US); Yihong LI, South San Francisco, CA (US); Julio MEDINA, South San Francisco, CA (US); Alok NERURKAR, South San Francisco, CA (US); Frederick SEIDL, South San Francisco, CA (US); David SPERANDIO, South San Francisco, CA (US); Tien WIDJAJA, South San Francisco, CA (US); Xiaodong WANG, South San Francisco, CA (US)

(73) Assignee: Tenaya Therapeutics, Inc., South San Francisco, CA (US)

(21) Appl. No.: 17/712,780

(22) Filed: Apr. 4, 2022

Related U.S. Application Data
(63) Continuation of application No. PCT/US2020/054134, filed on Oct. 2, 2020.
(60) Provisional application No. 62/910,278, filed on Oct. 3, 2019.

(51) Int. Cl. C07D 213/82; C07D 401/04; C07D 405/12; C07D 405/06; C07D 413/06; C07D 405/14; C07D 401/12; C07D 471/04; C07D 401/06; C07D 498/04; C07D 495/04; C07D 401/14; C07D 491/052

(52) U.S. Cl. CPC C07D 213/82; C07D 401/04; C07D 405/12; C07D 405/06; C07D 413/06; C07D 405/14; C07D 401/12; C07D 471/04; C07D 401/06; C07D 498/04; C07D 495/04; C07D 401/14; C07D 491/052

(57) ABSTRACT

Provided herein is a compound of Formula (I), or pharmaceutically acceptable salt thereof, wherein R¹, Y, X, and n are defined herein. Also provided herein are compositions comprising a compound of Formula (I) or pharmaceutically acceptable salt thereof, and methods of using a compound of Formula (I) or pharmaceutically acceptable salt thereof, e.g., in the treatment of heart disease.

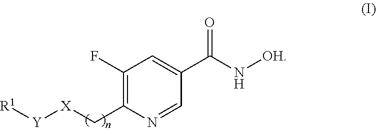

### CROSS-REFERENCE TO RELATED APPLICATIONS

This application claims the benefit of priority to U.S. Provisional Application Ser. No. 62/910,278, filed Oct. 3, 2019, the contents of which are hereby incorporated by reference in their entirety for all purposes.

### BACKGROUND

Histone deacetylase (HDAC) are a class of enzymes with deacetylase activity with a broad range of genomic and non-genomic substrates. There are eleven zinc-dependent HDAC enzymes classified based on sequence identity and catalytic activity.

Histone deacetylase inhibitors have been described and used in various therapeutic applications, including oncology, neurodegeneration, autoimmune disease, chemotherapy-induced peripheral neuropathy and cardiac indications. However, many HDAC inhibitors are nonspecific (i.e., they inhibit the activity of more than one HDAC with more or less the same affinity). When administered to humans, these so-called pan-HDAC inhibitors (e.g., SAHA and Panabinostat) exhibit significant adverse effects such as fatigue, nausea, diarrhea and thrombocytopenia. Thus, there is a need for FIDAC inhibitors that selectively target a pardcular MAC, such as HDAC6.

### SUMMARY

The present disclosure is directed to compounds that selectively inhibit HDAC6 activity and uses thereof in treating various diseases and disorders. For example, the present disclosure provides small molecules and compositions as well as therapeutic compositions and uses of specific small molecule compounds.

In one aspect, the present disclosure provides a compound of Formula (I) or pharmaceutically acceptable salt thereof:

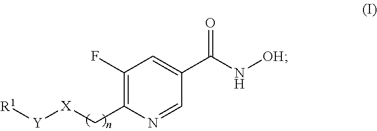

wherein:

n is 0 or 1;

X is O, NR⁴, or CR⁴R⁴′;

Y is a bond, CR²R³ or S(O)₂;

R¹ is selected from the group consisting of H, amido, carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)aryl, and —(CH₂)-heteroaryl; or

R¹ and R² taken together with the carbon atom to which they are attached form a carbocyclyl or heterocyclyl; or

R² and R³ taken together with the carbon atom to which they are attached form a carbocyclyl or heterocyclyl; and

R⁴ and R⁴′ are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-alyl, and —(CH₂)-heteroaiyl; or

R⁴ and R⁴′ taken together with the carbon atom to which they are attached form a carbocyclyl or heterocyclyl;

wherein each alkyl, carbocyclyl, heterocyclyl, aryl, and heteroaryl is independently optionally substituted with one or more substituents selected from the group consisting of halogen, haloalkyl, oxo, hydroxy, alikoxy, —OCH₃, —CO₂CH₃, —C(O)NH(OH), CH₃, morpholine, and —C(O)N-cyclopropyl.

In some embodiments, the present disclosure provides a compound of Formula (Ia) or pharmaceutically acceptable salt thereof:

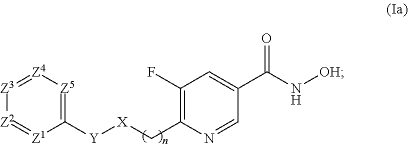

wherein:

n, X, and Y are as defined above for Formula (I); and

Z¹, Z², Z³, Z⁴ and Z⁵ are independently selected from N and CR⁵;

wherein R⁵ is independently selected from the group consisting of H, halogen, alkyl, haloalkyl, carbocyclyl, heterocyclyl, aryl, heteroary —CO₂H, —CO₂-alkyl, —O-alkyl, haloalkyl, —O-aryl. —O-heteroaryl, —SO²-alkyl, and —CN.

In some embodiments, the present disclosure provides a compound of Formula (fb) or pharmaceutically acceptable salt thereof:

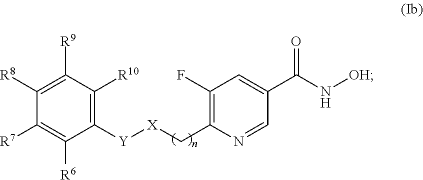

wherein:

n, X, and Y are as defined above for Formula (I); and

R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O— haloalkyl, —O-aryl, —O-heteroaryl, —SO₂-alkyl, and —CN.

In some embodiments, the present disclosure provides a compound of Formula (Ic) or pharmaceutically acceptable salt thereof:

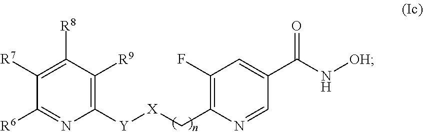

wherein:

n, X, and Y are as defined above for Formula (I); and

R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O— haloalkyl, —O-aryl, —O-heteroaryl, —SO₂-alkyl, and —CN.

In another aspect, the present disclosure provides a compound of Formula (II) or pharmaceutically acceptable salt thereof:

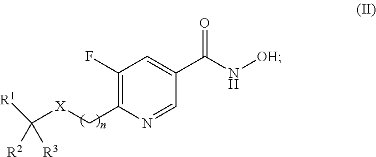

wherein:

n is 0 or 1;

X is NR⁴ or CR⁴R⁴′;

R¹ is selected from the group consisting of carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl, or

R² and R³ taken together with the carbon atom to which they are attached form a carbocyclyl or heterocyclyl; and

R⁴ and R⁴′ are independently selected from the group consisting of alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R⁴ and R⁴′ taken together with the carbon atom to which they are attached form a carbocyclyl or heterocyclyl; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

In some embodiments, the present disclosure provides a compound of Formula (IIa) or pharmaceutically acceptable salt thereof:

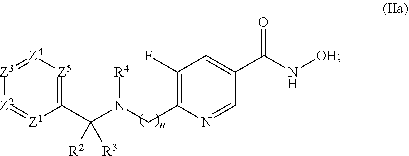

wherein:

n, R², R³, and R⁴ are as defined above in Formula (II);

Z¹, Z², Z³, Z⁴ and Z¹ are independently selected from N and CR⁵;

wherein R⁵ is independently selected from the group consisting of H, halogen, alkyl, haloalkyl, carbocyclyl, heterocyclyl, aryl, heteroaryl, —CO₂H, CO₂-alkyl, —O-alkyl, —O-haloalkyl, —O-aryl, —O-heteroaryl, —SO₂-alkyl, and —CN.

In some embodiments, the present disclosure provides a compound of Formula (IIb) or pharmaceutically acceptable salt thereof:

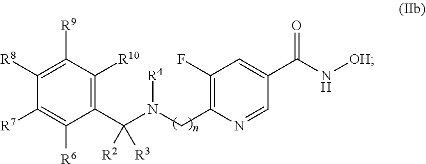

wherein:

n, R², R³, and R⁴ are as defined above in Formula (II);

R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O— haloalkyl, —O-heteroaryl, —SO₂-alkyl, and —CN.

In some embodiments, the present disclosure provides a compound of Formula (IIc) or pharmaceutically acceptable salt thereof:

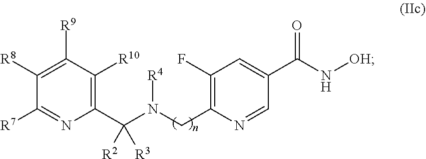

wherein:

n, R², R³, and R⁴ are as defined above in Formula (II);

R⁶, R⁷, R⁸, and R⁹ are independently selected from the group consisting of H, halogen, alkyl, haloalkyl, carbocyclyl, heterocyclyl, aryl, heteroaryl, —CO₂-alkyl, —O-alkyl, haloalkyl, —O-aryl, —O-heteroaryl, —SO₂-alkyl, and —CN.

In some embodiments, the present disclosure provides a compound of Formula (III) or pharmaceutically acceptable salt thereof:

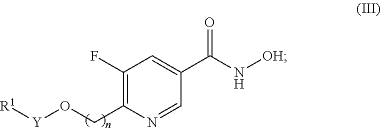

wherein:

n is 0 or 1;

Y is a bond or CR²R³;

R¹ is selected from the group consisting of H, carbocyclyl, heterocyclyl, aryl, and heteroaryl;

R² and R³ are independently selected from the group consisting of H, halogen, alkyl, carbocyclyl, heterocyclyl, aryl, heteroaryl, carbocyclyl, —(CH₂)-carbocyclyl, —(CH₂)-heterocyclyl, —(CH₂)-aryl, and —(CH₂)-heteroaryl; or

R¹ and R² taken together with the carbon atom to which they are attached form a carbocyclyl or heterocyclyl; or

R² and R³ taken together with the carbon atom to which they are attached form a carbocyclyl or heterocyclyl; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

In some embodiments, the present disclosure provides a compound of Formula (IV) or pharmaceutically acceptable salt thereof:

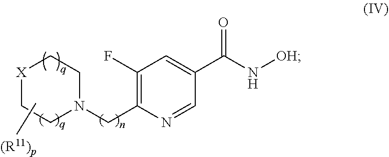

wherein:

n is 0 or 1;

p is 0, 1, 2, 3, or 4;

q is each independently 0, 1, or 2;

X is O, S(O)₂, NR¹², or CHR¹²;

R¹¹ is each independently H, F, alkyl, or oxo; or

two adjacent R¹¹ taken together with the carbon atoms to which they are attached form an aryl, heteroaryl, or heterocyclyl ring; or

two non-adjacent R¹¹ taken together with the atoms to which they are attached form a carbocyclyl or heterocyclyl ring;

R¹² is selected from the group consisting of alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R¹¹ and R¹² taken together with the carbon and/or nitrogen atoms to which they are attached form an aryl or heteroaryl ring; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

In some embodiments, the present disclosure provides therapeutic methods comprising use of the compounds disclosed herein (i.e., Formula (I), Formula (Ia), Formula (II)), Formula (Ic), Formula (II), Formula (IIa), Formula (IIb), Formula (IIc), Formula (III), and Formula (IV)) in treating patients suffering from aberrant cell proliferative disorders, β-amyloid protein aggregation, polyglutamine protein aggregation, neurodegeneration, stroke, psychiatric disorders, depression, autoimmune disease, chemotherapy-induced neuropathy, Charcot-Marie-Tooth disease, idiopathic pulmonary fibrosis, erectile dysfunction, hypertension, muscular dystrophy, and/or cardiac diseases or disorders. Proliferative disorders include, but are not limited to, malignant gliomas, breast cancer, basal cell carcinoma, medulloblastomas, neuroectodermal tumors, and ependvinomas. Cardiac diseases or disorders that can be treated with the compounds of the present disclosure include, but art not limited to, coronary heart disease, cardiomyopathy, endocarditis, congenital cardiovascular defects, congestive heart failure, dilated cardiomyopathy, hypertrophic cardiomyopathy, valvular heart disease, myocardial infarction, congestive heart failure, long QT syndrome, atrial arrhythmia, ventricular arrhythmia, diastolic heart failure, systolic heart failure, cardiac valve disease, cardiac valve calcification, left ventricular non-compaction, ventricular septal defect, and ischemia.

### Definitions

While the following terms are believed to be well understood by one of ordinary skill in the art, the following definitions are set forth to facilitate explanation of the presently disclosed subject matter.

The term “a” or “an” refers to one or more of that entity; for example, “an HDAC6 inhibitor” refers to one or more HDAC6 inhibitors or at least one HDAC6 inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, sec-propyl, n-butyl, i-butyl, see-butyl, 1-butyl, n-pentyl, n-hexyl, n-heptyl, n-octyl, n-nonyl, n-decyl, n-undecyl, and n-dodecyl, Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included, An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls, A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₅ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally, substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethenylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenantlitylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R_{b} is an alkylene, alkenylene, or alkynylene group as defined above and R_{d} is a carbocyclyl radical as defined above. Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enol and the like, Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclycl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazotyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ Where R_{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amities, amides, alkylamines, dialkylamines, arylamines, alkylarylamines, diarylamines, N-oxides, imides, and enamines; a silicon atom in groups such as trialkylsityl groups, dialkylarylsityl groups, alkyldiarylsityl groups, and triaryisilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂NR_{g}Rₕ, In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroaryl alkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

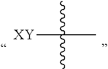

indicates that the chemical entity “XY” is bonded to another chemical entity via the point of attachment bond. Furthermore, the specific point of attachment to the non-depicted chemical entity can be specified by inference. For example, the compound CH₃-R³, wherein R³ is H or

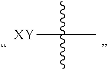

infers that when R³ is “XY”, the point of attachment bond is the same bond as the bond by which R³ is depicted as being bonded to CH₃.

### DETAILED DESCRIPTION

Histone deacetylases (“HDAC”) are a class of enzymes with deacetylase activity with a broad range of genomic and non-genomic substrates. There are eleven Zinc-dependent HDAC enzymes classified based on sequence identity and catalytic activity (Habed and et al., 2009).

Histone deacetylase inhibitors have been described as a therapeutic agents in oncology (Poon and Eom, 2016), neurodegeneration (Butler et at, 2010) autoimmune disease (Choi et al., 2018), chemotherapy-induced peripheral neuropathy (Krukowski et al., 2017) and cardiac indications (Zhang et al., 2002). Given the role of nuclear HDACs on regulating gene transcription, inhibition of these class of targets is known to have pleiotropic effects in various cell types; most notably resulting in cell toxicities. Therefore, limiting the toxicity of pan-HDAC inhibitors has been a major obstacle in wide-spread utilization for this class of compounds. In addition, significant adverse effects of pan-HDAC inhibitors (e.g. SAHA and Panabinostat) has been observed in the clinic including fatigue, nausea, diarrhea and thrombocytopenia. (Subramanian et al., 2010).

In the cardiac-indication space, most studies have utilized pan-HDAC inhibitors (e.g. SAHA, TSA and Givinostat) for the treatment of pressure-overload rodent models including transverse aortic constriction (TAC) (Cao et al., 2011), hypertension in Dahl salt-sensitive rats (Jeong et al., 2018) and myocardial infarction(Nagata et al., 2019). In addition, HDAC6-selective inhibitors have been used to ameliorate the effects of pressure overload in rodent models (Demos-Davies et al., 2014) and provide protection against proteotoxicity in a transgenic cardiomyopathy mouse model (McLendon et al., 2014).

HDAC6 belongs to the class IIb enzyme and contains two catalytic domains, a ubiquitin binding domain and a cytoplasmic retention domain (Haberland et al., 2009). HDAC6 is predominately a cytoplasmic enzyme and its best-characterized substrates include tubulin, HSP90 and cortactin (Brindisi et al., 2019).

Pharmacological inhibition of HDAC6 blocks its deacetylase activity, thus resulting in hyperacetylation of its substrates, most notably tubulin (Hubbert et al., 2002).

HDAC6-selective inhibitors are known to have reduced cytotoxicity due to the cytoplasmic nature of HDAC6 substrates and reduced effects on nuclear targets (including H3K9 and c-MYC) and on global transcription (Nebbioso et al, 2017).

Hydroxamic acids are zinc chelators and have been used extensively in the development of pan- and HDAC-selective inhibitors. However, most hydroxamic-acid based HDAC inhibitors either lack the desired selectivity or show poor bioavailability with a poor pharmacokinetic profile (Butler et al., 2010; Santo et al., 2012).

The present disclosure provide hydroxamic acid compounds that, in some embodiments, selectively inhibit HDAC6.

### Compounds of Formulas (I)-(III)

In one aspect, the present disclosure provides a compound of Formula (I) or pharmaceutically acceptable salt thereof:

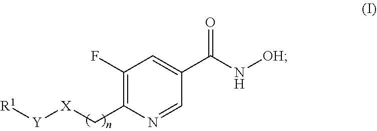

wherein:

n is 0 or 1;

X is O, —NR⁴, or CR⁴R⁴′;

Y is a bond, CR²R³ or S(O)₂;

R¹ is selected from the group consisting of H, amido, carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R¹ and R² taken together with the carbon atom to which they are attached form a carbocyclyl or heterocyclyl; or

R² and R³ taken together with the carbon atom to which they are attached form a carbocyclyl or heterocyclyl; and

R⁴ and R⁴′ are each independently selected from the group consisting of H, alkyl, —CO₂-alkyl, carbocyclyl, heterocyclyl, aryl, heteroaryl, carbocyclyl, —(CH₂)-heterocyclyl, —(CH₂)-aryl, and —(CH₂)-heteroaryl; or

R⁴ and R⁴′ taken together with the carbon atom to which they are attached form a carbocyclyl or heterocyclyl;

wherein each alkyl, carbocyclyl, heterocyclyl, aryl, and heteroaryl is independently optionally substituted with one or more substituents selected from the group consisting of halogen, haloalkyl, hydroxy, alkoxy, —OCH₃, CO₂CH₃, —C(O)NH(OH), —CH₃, morpholine, and —C(O)N-cyclopropyl.

In one aspect, the present disclosure provides a compound of Formula (I) or pharmaceutically acceptable salt thereof:

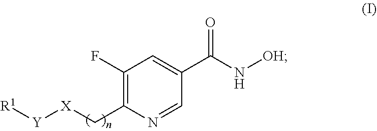

wherein

n is 0 or 1;

X is O, NR⁴, or CR⁴R⁴′;

Y is a bond, CR²R³ or S(O)₂;

R¹ is selected from the group consisting of H, amido, carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂) heteroaryl; or

R¹ and R² taken together with the carbon atom to which they are attached form a carbocyclyl or heterocyclyl; or

R² and R³ taken together with the carbon atom to which they are attached form a carbocyclyl or heterocyclyl; and

R⁴ and R⁴′ are each independently selected from the group consisting of H, alkyl, carbocyclyl, heterocyclyl, aryl, heteroaryl, —(CH₂) carbocyclyl, —(CH₂)-heterocyclyl, —(CH₂)-aryl, and (CH₂)-heteroaryl; or

R⁴ and R⁴′ taken together with the carbon atom to which they are attached form a carbocyclyl or heterocyclyl;

wherein each alkyl, carbocyclyl, heterocyclyl, aryl, and heteroaryl is independently optionally substituted with one or more halogen, haloalkyl, oxo, hydroxy, alkoxy, —OCH₃, —CO₂CH₃, —C(O)NH(OH), and —CH₃.

In some embodiments, a compound of Formula (I) or a pharmaceutically acceptable salt thereof is provided:

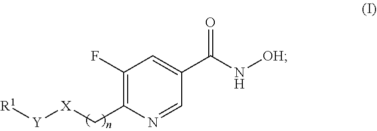

wherein:

n is 0 or 1;

X is NR⁴ or CR⁴R⁴′;

Y is CR²R³ or S(O)₂;

R¹ is selected from the group consisting of carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂) heterocyclyl, —(CH₂)-aryl, and —(CH₂)-heteroaryl, or

R² and R³ taken together with the carbon atom to which they are attached form a carbocyclyl or heterocyclyl, each of which is optionally substituted; and

R⁴ and R⁴′ are independently selected from the group consisting of H, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R⁴ and R⁴′ taken together with the carbon atom to which they are attached form a carbocyclyl or heterocyclyl, each of which is optionally substituted; and

wherein each alkyl, carbocyclyl, heterocyclyl, aryl, and heteroaryl is independently optionally substituted with one or more substituents selected from the group consisting of halogen, oxo, hydroxy, alkoxy, —CO₂CH₃ and —CH₃.

In some embodiments of Formula (I), n is 1. In some embodiments, n is 0.

In some embodiments of Formula (I), X is NR⁴ or CR⁴R⁴′. In some embodiments, X is NR⁴ or O. In some embodiments, X is NR⁴. In some embodiments, X is CR⁴R⁴′. In some embodiments, X is O.

In some embodiments of Formula (I), X is NR⁴, and R⁴ is H.

In some embodiments of Formula (I), Y is a bond or CR²R³. In some embodiments, Y is S(O)₂ or CR²R³. In some embodiments, Y is a bond. In some embodiments, Y is CR²R³. In some embodiments, Y is S(O)₂.

In some embodiments of Formula (I), X is NR⁴ and Y is CR²R³. In some embodiments, X is NR⁴ and Y is S(O)₂. In some embodiments, X is NR⁴ and Y is a bond. In some embodiments, X is CR⁴R⁴′ and Y is CR²R³. In some embodiments, X is CR⁴R⁴′ and Y is a bond. In some embodiments, X is O and Y is CR²R³. In some embodiments, X is O and Y is a bond.

In some embodiments of Formula (I), R¹ is selected from the group consisting of amido, carbocyclyl, heterocyclyl, aryl, and heteroaryl. In some embodiments, R¹ is selected from the group consisting of carbocyclyl, heterocyclyl, aryl, and heteroaryl. In some embodiments, R¹ is selected from the group consisting of H, cyclopropyl, phenyl, 6-membered heterocyclyl, 8-10 membered fused bicyclic heterocyclyl and 11-13 membered fused tricyclic heterocyclyl, wherein each heterocyclyl contains 1-3 heteroatoms selected from the group consisting of N, O, and S(O)_{w} (wherein w is 0, 1, or 2). In some embodiments, each cyclopropyl, phenyl, and heterocyclyl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl. In some embodiments, R¹ is a heteroaryl selected from the group consisting of pyrimidinyl, pyridinyl, pyridazine, and pyrazine. In some embodiments, R¹ is pyridinyl. In some embodiments, R¹ is phenyl. In some embodiments, R¹ is H. In some embodiments, R¹ is cyclopropyl. In some embodiments, R¹ is selected from the group consisting of pyridinyl, hydrogen, cyclopropyl, and phenyl.

In some embodiments of Formula (I), X is NR⁴. Y is CR²R³, and R¹ is aryl or heteroaryl. In some embodiments, X is NR⁴, Y is CR²R³, and R¹ is aryl. In some embodiments, X is NR⁴, Y is CR²R³, and R¹ is heteroaryl. In some embodiments, n is 0. In some embodiments, n is 1.

In some embodiments of Formula (I), X is Y is a bond, and R¹ is H. In some embodiments, X is CR⁴R⁴′, Y is a bond, and R¹ is H. In some embodiments, X is O, Y is CR²R³, and R¹ is H. In some embodiments, n is 0. In some embodiments, n is 1.

In some embodiments of Formula (I), R¹ and R² taken together with the carbon atom to which they are attached form a C₃₋₁₂ carbocyclyl. In some embodiments, the C₃₋₁₂ carbocyclyl is a propyl ring. In some embodiments, the C₃₋₁₂ carbocyclyl is a cyclobutyl ring. In another embodiment, the C₃₋₁₂ carbocyclyl is an indane ring. In some embodiments of Formula (I), R¹ and IC taken together with the carbon atom to which they are attached form a C₃₋₁₂ heterocyclyl. In some embodiments, the C₃₋₁₂ heterocyclyl is an oxetanyl ring.

In some embodiments of Formula (I), R² and R³ are independently selected from the group consisting of F, C₁₋₆ alkyl, C₃₋₆ cycloalkyl, —(CH₂)—C₃₋₆ cycloalkyl, 4- to 6-membered heterocyclyl, and —(CH₂)-(4- to 6-membered heterocyclyl). In some embodiments, R³ is C₁₋₆ alkyl optionally substituted with alkoxy. In some embodiments, R³ is Cis alkyl. In some embodiments, R² and R³ taken together with the carbon atom to which they are attached form a C₃₋₆ cycloalkyl. In some embodiments, R² and R³ taken together with the carbon atom to which they are attached. form a cyclopropyl, cyclobutyl, cyclopentyl or cyclohexyl. In some embodiments, R² and R³ taken together with the carbon atom to which they are attached form a cyclopropyl, cyclobutyl, or cyclohexyl. In some embodiments, R² and R³ taken together with the carbon atom to which they are attached form a cyclopropyl. In some embodiments, R² and R³ taken together with the carbon atom to which they are attached form a 4- to 6-membered heterocyclyl. In some embodiments, R² and R³ taken together with the carbon atom to which they are attached form an azetidinyl, oxetanyl, pyrrolidinyl, pipezidinyl piperazinyl, morpholinyl, thiomorpholinyl, tetrahydrofuranyl, or tetrahydropyranyl. In some embodiments, R² and R³ taken together with the carbon atom to which they are attached form an oxetanyl, cyclopropyl, cyclobutyl, cyclopentyl, pyrrolidinyl, and piperidinyl, each of which is optionally substituted with one or more substituents selected from the group consisting of fluoro, —OCH₃, —CH₃ and oxo.

In some embodiments of Formula (I), R² is H and R³ is C₃₋₆ cycloalkyl. In some embodiments, R² is H and R³ is cyclopropyl. In some embodiments, R² and R³ are C₁₋₆ alkyl. In some embodiments, R² and R³ are methyl.

In some embodiments of Formula (I), R¹ is C₃₋₆ cycloalkyl or aryl, R² is H, and R³ is C₃₋₆ cycloalkyl or aryl. In some embodiments, R¹ is aryl, R² is H, and R³ is C₃₋₆ cycloalkyl or aryl. In some embodiments, R¹ is aryl, R² is H, and R³ is C₃₋₆ cycloalkyl or C₁₋₅ alkyl. In some embodiments, R¹ is aryl, R² is H, and R³ is C₃₋₆ cycloalkyl. In some embodiments, R¹ is C₃₋₆ cycloalkyl, R² is H, and R³ is C₃₋₆ cycloalkyl or aryl. In some embodiments, R¹ is C₃₋₆ cycloalkyl, R² is H, and R³ is C₃₋₆ cycloalkyl or C₁₋₅ alkyl. In some embodiments, R¹ is C₃₋₆ cycloalkyl, R² is H, and R³ is C₃₋₆ cycloalkyl. In some embodiments, the aryl is phenyl and C₃₋₆ cycloalkyl is cyclopropyl.

In some embodiments of Formula (I), R⁴ is selected from the group consisting of H, alkyl, carbocyclyl, heterocyclyl, —(CH₂)-carbocyclyl, and —(CH₂)-heterocyclyl. In some embodiments, R⁴ is H or alkyl. In some embodiments, R⁴ is H. In some embodiments, R⁴ is —(CH₂)-heterocyclyl. In some embodiments, R⁴ is —(CH₂)-oxetane. In some embodiments, R⁴ is alkyl. In some embodiments, the alkyl is C₁₋₅ alkyl. In some embodiments, R⁴ is methyl. In some embodiments, R⁴ is ethyl. In some embodiments, R⁴ is isopropyl. In some embodiments, R⁴ is —C(O)(CH₃).

In some embodiments of Formula (I), R⁴ and R⁴′ are each H. In some embodiments, R⁴ and R⁴′ are each alkyl. In some embodiments, R⁴ and R⁴′ are each methyl. In some embodiments, R⁴ and R⁴′ taken together with the carbon atom to which they are attached form a C₃₋₆ cycloalkyl. In some embodiments, R⁴ and R⁴′ taken together with the carbon atom to which they are attached form a cyclopropyl.

In some embodiments, the compound of Formula (I) is selected from the group consisting of:

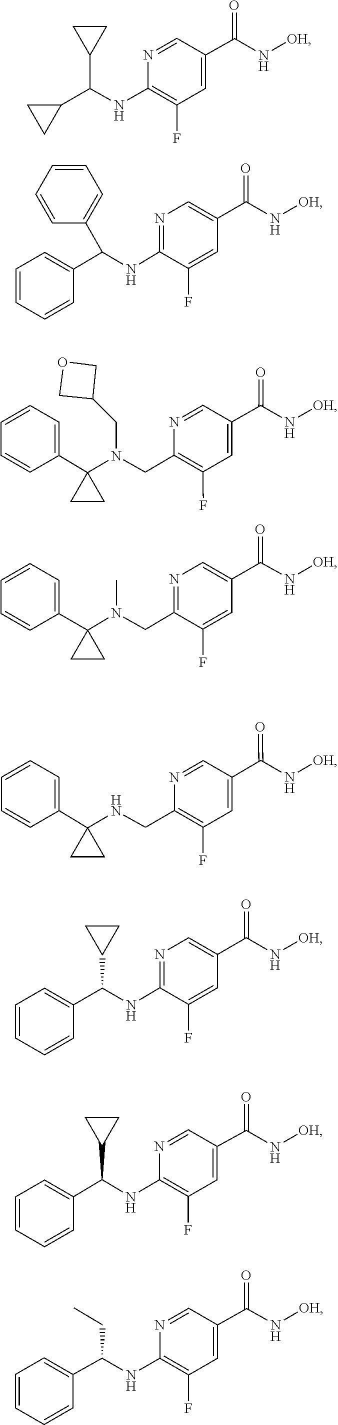

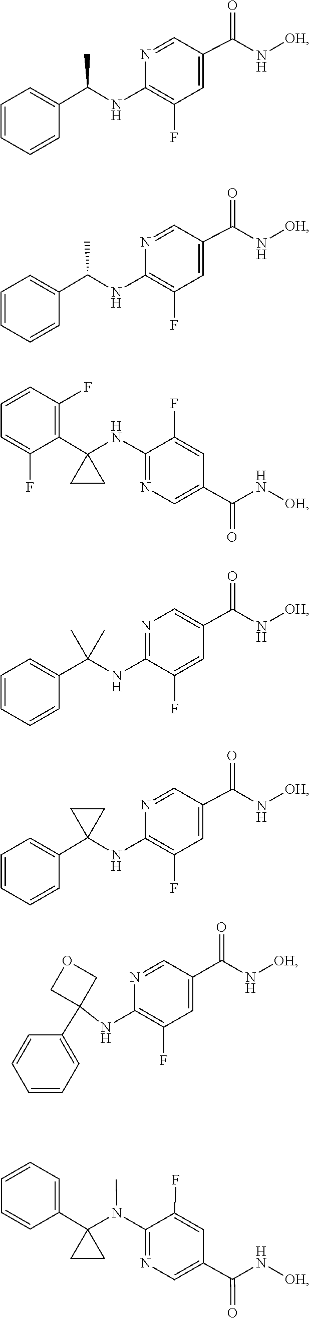

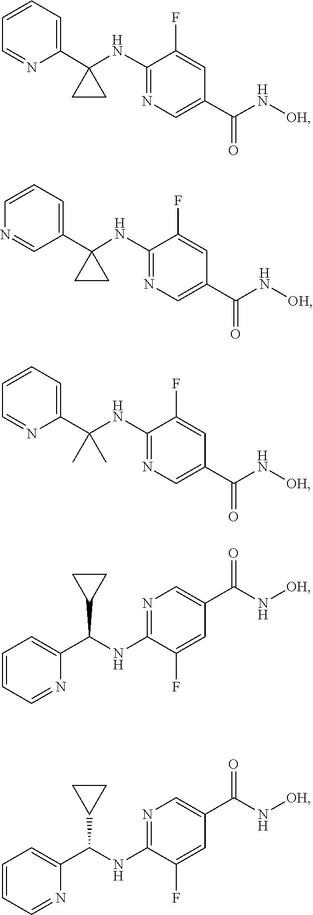

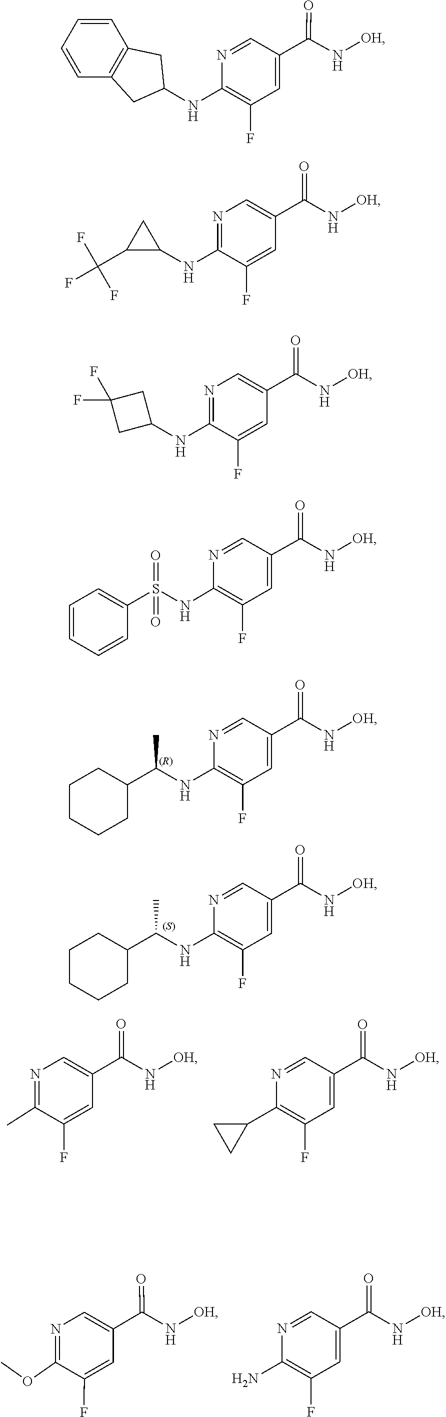

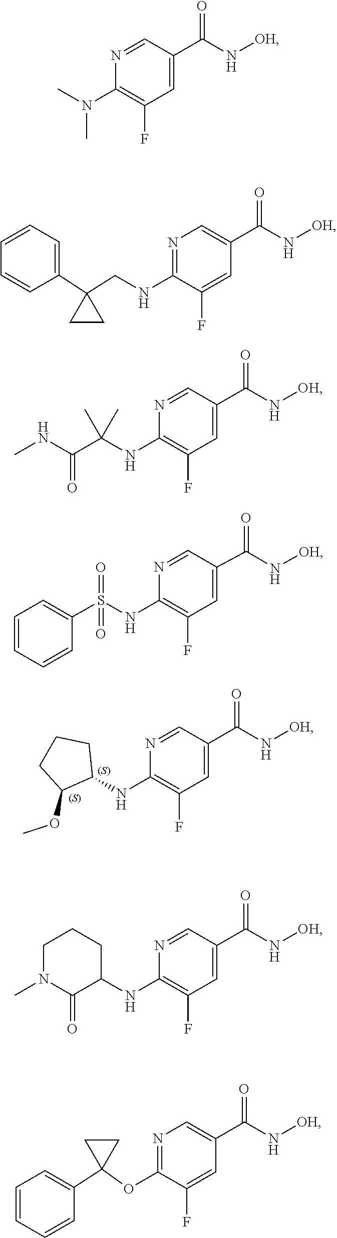

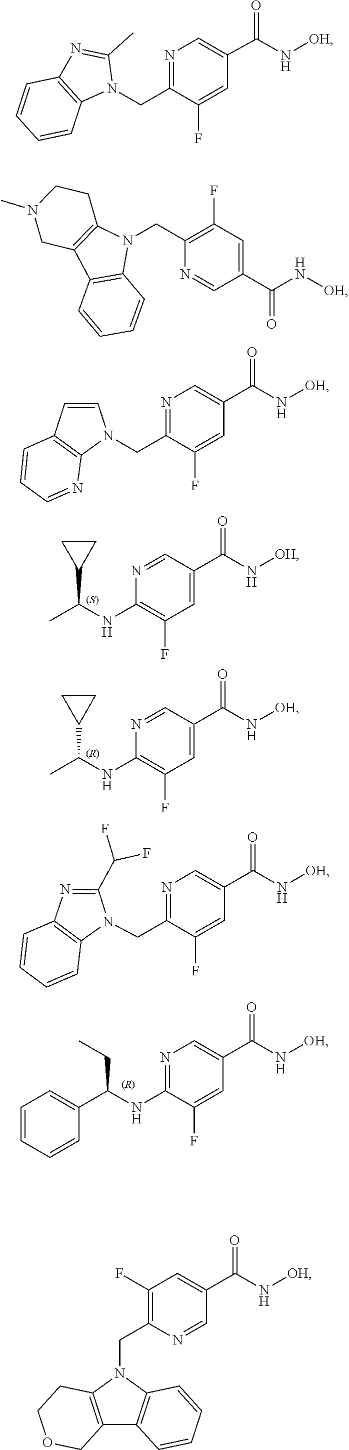

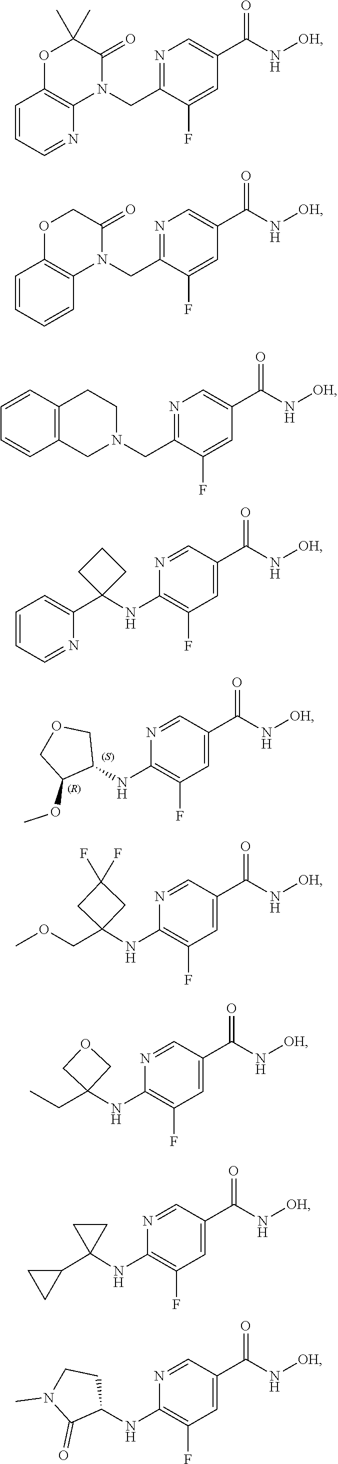

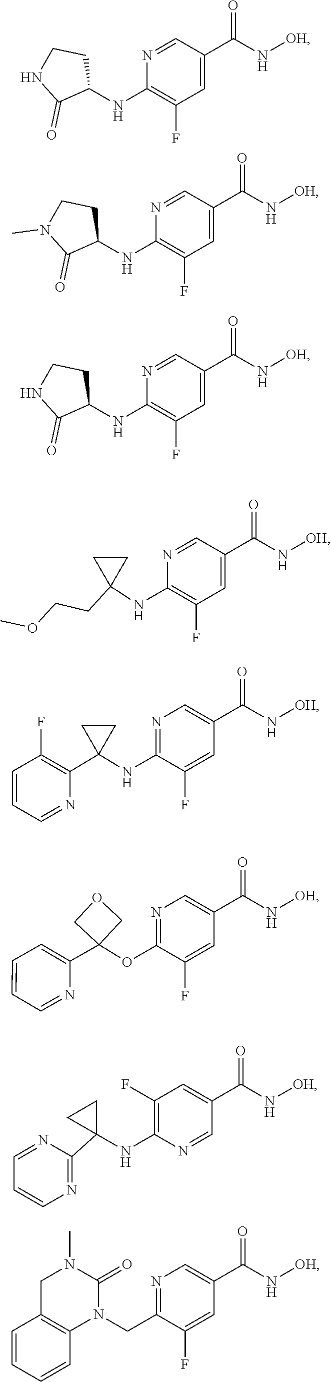

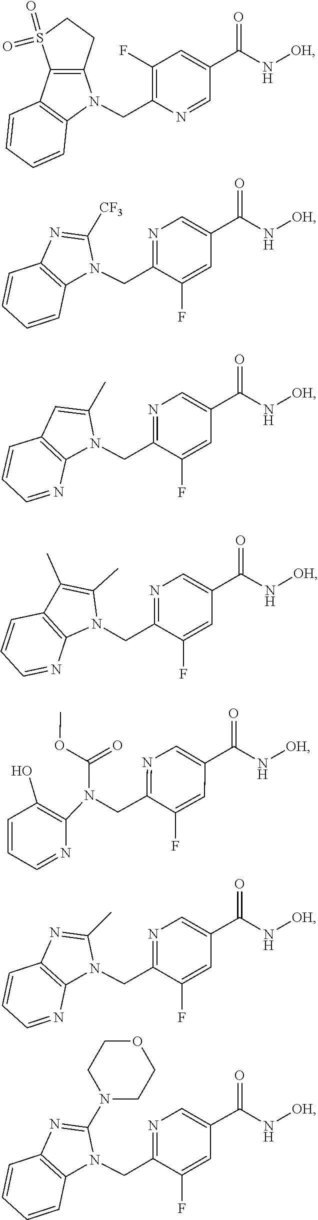

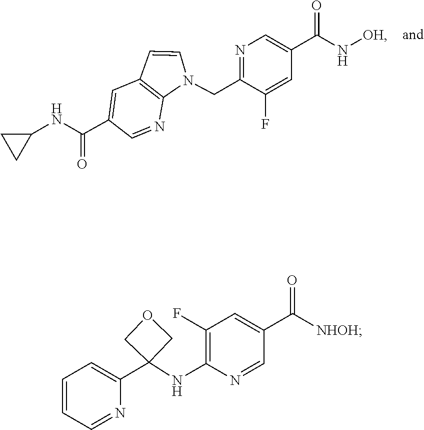

or a pharmaceutically acceptable salt thereof.

In some embodiments, the present disclosure provides a compound of Formula (Ia) or pharmaceutically acceptable salt thereof:

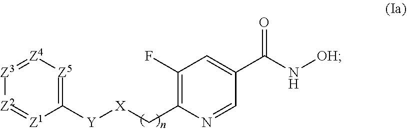

wherein:

n, X, and Y are as defined above for Formula (I); and

Z¹, Z², Z³, Z⁴ and Z⁵ are independently selected from N and CR⁵;

wherein R⁵ is independently selected from the group consisting of H, halogen, alkyl, haloalkyl, carbocyclyl, heterocyclyl, aryl, heteroaryl, —CO₂H, —CO₂-alkyl, —O-alkyl, haloalkyl, —O-aryl, —O-heteroaryl, SO₂ alkyl and

In some embodiments of Formula (Ia), Z¹, Z², Z³, Z⁴ and Z⁵ are CR⁵. In some embodiments, Z¹ is N and Z², Z³, Z⁴ and Z⁵ are CR⁵. In some embodiments, Z² is N and Z¹, Z³, Z⁴ and Z⁵ are CR⁵. In some embodiments, Z³ is N and Z¹, Z², Z⁴ and Z⁵ are CR⁵. In some embodiments, one of Z³, Z⁴ and Z⁵ is N. In some embodiments, two of Z¹, Z², Z³, Z⁴ and Z⁵ are N. In some embodiments, Z¹ and Z⁵ are each N.

In some embodiments of Formula (Ia), R⁵ is independently selected from H, halogen, alkyl, alkoxy, and haloalkyl. In some embodiments, R⁵ is independently selected from H and halogen. In some embodiments, R⁵ is independently selected from H and fluoro.

In some embodiments, the present disclosure provides a compound of Formula (Ib) or pharmaceutically acceptable salt thereof:

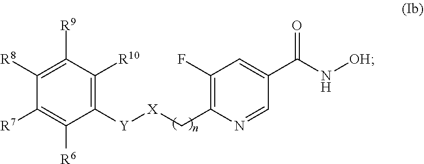

wherein:

n, X, and Y are as defined above for Formula (I); and

R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O— haloalkyl, —O-aryl, —O-heteroaryl, —SO₂-alkyl, and —CN.

In some embodiments of Formula (Ib), R⁶, R⁷, R⁸, R⁹, and R¹⁰ are independently selected from the group consisting of H, halogen, alkyl, haloalkyl, —CO₂-alkyl, —O-alkyl, —O-haloalkyl, —O-aryl, —O-heteroaryl, —SO₂-alkyl, and —CN. In some embodiments, R⁶, R⁷, R⁸, R⁹, and R¹° are independently selected from the group consisting of H, halogen, alkyl, haloalkyl, —O-alkyl, and —O-haloalkyl. In some embodiments, R⁶, R⁷, R⁸, R⁹, and R¹⁰ are independently selected from the group consisting of H and halogen. In some embodiments, R⁶ and R¹⁰ are halogen and R⁷, R⁸, and R⁹ are H. In some embodiments, R⁶ and R¹⁰ are fluoro and R⁷, R⁸, and R⁹ are H.

In some embodiments, the present disclosure provides a compound of Formula (Ic) or pharmaceutically acceptable salt thereof:

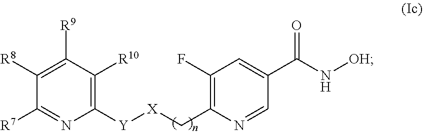

wherein:

n, X, and Y are as defined above for Formula (I); and

R⁶, R⁷, R⁸, and R⁹ are independently selected from the group consisting of H, halogen, alkyl, hydroxyl, haloalkyl, carbocyclyl, heterocyclyl, aryl, heteroaryl, —COAL, —CO₂-alkyl, —O-alkyl, —O-haloalkyl, —O-heteroaryl, —SO₂-alkyl, and —CN.

In some embodiments of the compound of Formula (Ic), R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂-alkyk, —O-alkyl, —O-haloalkyl, —O-heteroaryl, —SO₂-alkyl, and —CN.

In some embodiments of Formula (Ic), R⁶, R⁷, R⁸, and R⁹ are independently selected from the group consisting of H, halogen, alkyl, haloalkyl, —CO₂H, —CO₂-alkyl, —O-alkyl, —O-haloalkyl, —O-aryl, —O-heteroatyl, —SO₂-alkyl, and —CN. In some embodiments, R⁶, R⁷, R⁸, and R⁹ are independently selected from the group consisting of H, halogen, alkyl, haloalkyl, —O-alkyl, and —O-haloalkyl. In some embodiments, R⁶, R⁷, R⁸, and R⁹ are independently selected from the group consisting of H and halogen. In some embodiments, R⁶ is halogen and R⁷, R⁸, and R⁹ are H. In some embodiments, R⁶ is fluoro and R⁷ R⁸, and R⁹ are H.

In some embodiments of Formulas (I)-(Ic), each optionally substituted alkyl is independently an optionally substituted C₁₋₆ alkyl. In further embodiments, the C₁₋₆ alkyl is selected from the group consisting of methyl, ethyl, n-propyl, isopropyl, n-butyl, isobutyl, tert-butyl, amyl, and isoamyl. In some embodiments, the C₁₋₆ alkyl is Me or Et. In some embodiments, the C₁₋₆ alkyl is a C₁₋₆ haloalkyl. In further embodiments, the C₁₋₆ haloalkyl is selected from the group consisting of —CF, —CHF₂, —CH₂F, and —CHBr₂. In some embodiments, the C₁₋₆ haloalkyl is CF₃. In some embodiments, the C₁₋₆ haloalkyl is CHF₂.

In some embodiments of Formulas (I)-(Ic), each optionally substituted carbocyclyl is independently an optionally substituted C₃₋₁₂ cycloalkyl. In some embodiments, the carbocyclyl is a C₃₋₆ cycloalkyl. In some embodiments, the cycloalkyl is selected from the group consisting of cyclopropyl, cyclobutyl, cyclopentyl, and cyclohexyl.

In some embodiments of Formulas (I)-(Ic), each optionally substituted heterocyclyl is independently an optionally substituted 3-12 membered heterocycloalkyl having 1 or 2 heteroatoms independently selected from N, O, and S. In some embodiments, each optionally, substituted heterocyclyl is independently an optionally substituted 3-6 membered heterocycloalkyl having 1 or 2 heteroatoms independently selected from N, O, and S. In further embodiments, the heterocycloalkyl is an optionally substituted 5-membered or 6-membered heterocycle having 1 or 2 heteroatoms independently selected from N, O, and S. In some embodiments, the heterocyclyl is selected from the group consisting of aziridinyl, azetidinyl, pyrrolidinyl, piperidinyl, morpholinyl, and thiomorpholinyl.

In some embodiments of Formulas (I)-(Ic), each optionally substituted aryl is independently a C₆₋₁₂ aryl. In further embodiments, the C₆₋₁₂ aryl is an optionally substituted phenyl.

In some embodiments of Formulas (I)-(Ic), each optionally substituted heteroaryl is independently a 5-12 membered heteroaryl having 1, 2, or 3 heteroatoms independently selected from N, O, and S. In some embodiments, each optionally substituted heteroaryl is independently a 5-12 membered heteroaryl having 3 heteroatoms independently selected from N, O, and S. In some embodiments, each optionally substituted heteroaryl is independently a 5-12 membered heteroaryl having 2 heteroatoms independently selected from N, O, and S. In some embodiments, each optionally substituted heteroaryl is independently a 5-12 membered heteroaryl having 1 heteroatom independently selected from N, O, and S. In further embodiments, each optionally substituted heteroaryl is an optionally substituted 5-membered or 6-membered heteroaryl having 1 heteroatom independently from N, O, and S. In some embodiments, each heteroaryl is independently selected from the group consisting of tetrazole, oxadiazole, thiadiazole, imidazole, pyrazole, thiazole, or oxazole, each of which is optionally substituted. In some embodiments, the heteroaryl is tetrazole. In some embodiments, the heteroaryl is oxadiazole.

In one aspect, the present disclosure provides a compound of Formula (II) or pharmaceutically acceptable salt thereof:

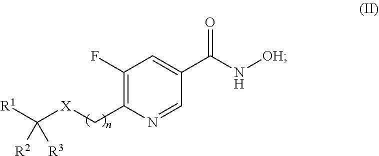

wherein:
n is 0 or 1;
X is NR⁴ or CR⁴R⁴′;

R¹ is selected from the group consisting of carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂) aryl, and —(CH₂)-heteroaryl, or

R² and R³ taken together with the carbon atom to which they are attached form a carbocyclyl or heterocyclyl; and

R⁴ and R⁴′ are independently selected from the group consisting of H, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl, or

R⁴ and R⁴′ taken together with the carbon atoms which they are attached form a carbocyclyl or heterocyclyl; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

In some embodiments of Formula (II), n is 1. In some embodiments, n is 0.

In some embodiments of Formula (II), X is NR⁴. In some embodiments, X is CR⁴R^{4′.}

In some embodiments of Formula (II), R¹ is selected from the group consisting of carbocyclyl, heterocyclyl, aryl, and heteroaryl. In some embodiments, R¹ is a heteroaryl selected from the group consisting of pyrimidinyl, pyridinyl, pyridazine, and pyrazine. In some embodiments, R¹ is pyridinyl. In some embodiments, R¹ is phenyl.

In some embodiments of Formula (II), X is NR⁴ and R¹ is aryl or heteroaryl. In some embodiments, X is NR⁴ and R¹ is aryl. In some embodiments, X is NR⁴ and R¹ is heteroaryl. In some embodiments, X is NR⁴ and R¹ is carbocyclyl. In some embodiments, X is —NR¹ and R¹ is heterocycloalkyl. In some embodiments, n is 0. In some embodiments, n is 1.

In some embodiments of Formula (II), R¹ and R² taken together with the carbon atom to which they are attached form a C₃₋₁₂ carbocyclyl. In some embodiments, the C₃₋₁₂ carbocyclyl is a propyl ring. In some embodiments, the C₃₋₁₂ carbocyclyl is a cyclobutyl ring. In another embodiment, the C₃₋₁₂ carbocyclyl is an indane ring.

In some embodiments of Formula (II), R² and R³ are independently selected from the group consisting of H, F, C₁₋₆ alkyl, C₃₋₆ cycloalkyl, cycloalkyl, 4- to 6-membered heterocyclyl, and —(CH₂)—(4- to 6-membered heterocyclyl). In some embodiments, R² and R³ taken together with the carbon atom to which they are attached form a C₃₋₆ cycloalkyl. In some embodiments, R² and R³ taken together with the carbon atom to which they are attached form a cyclopropyl, cyclobutyl, or cyclohexyl. In some embodiments, R² and R³ taken together with the carbon atom to which they are attached form a cyclopropyl. In some embodiments, R² and R³ taken together with the carbon atom to which they are attached form a 4- to 6-membered heterocyclyl. In some embodiments, R² and R³ taken together with the carbon atom to which they are attached form a azetidinyl, pyrrolidinyl, piperidinyl, piperazinyl, morpholinyl, thiomorpholinyl, tetrahydrofuranyl, or tetrahydropyranyl.

In some embodiments of Formula (II), R² is H and R³ is C₃₋₆ cycloalkyl. In some embodiments, R² is H and R is cyclopropyl. In some embodiments, R² and R³ are C₁₋₆ alkyl. In some embodiments, R² and R³ are methyl.

In some embodiments of Formula (II), R¹ is C₃₋₆ cycloalkyl or aryl, R² is H, and R³ is C₃₋₆ cycloalkyl or aryl. In some embodiments, R¹ is aryl, R² is H, and R³ is C₃₋₆ cycloalkyl or aryl. In some embodiments, R¹ is aryl, R² is H, and R³ is C₃₋₆ cycloalkyl or C₁₋₅ alkyl. In some embodiments, R¹ is aryl, R² is H, and R³ is C₃₋₆ cycloalkyl. In some embodiments, is C₃₋₆ cycloalkyl, R² is H. and R³ is C₃₋₆ cycloalkyl or aryl. In some embodiments, R¹ is C₃₋₆ cycloalkyl, R² is H, and R³ is C₃₋₆ cycloalkyl or C₁₋₅ alkyl. In some embodiments, R¹ is C₃₋₆ cycloalkyl, R² is H, and R³ is C₃₋₆ cycloalkyl. In some embodiments, the aryl is phenyl and C₃₋₆ cycloalkyl is cyclopropyl.

In some embodiments of Formula (II), R⁴ is selected from the group consisting of H, alkyl, carbocyclyl, heterocyclyl, —(CH₂)-carbocyclyl, and —(CH₂)-heterocyclyl. In some embodiments, R⁴ is H or alkyl. In some embodiments, R⁴ is H. In some embodiments, R⁴ is —(CH₂)-heterocyclyl. In some embodiments, R⁴ is —(CH₂)-oxetane. In some embodiments, R⁴ is alkyl. In some embodiments, the alkyl is C₁₋₅ alkyl. In some embodiments, R⁴ is methyl. In some embodiments, R⁴ is ethyl. In some embodiments, R⁴ is isopropyl.

In some embodiments of Formula (II), R⁴ and R⁴′ are each H. In some embodiments, R⁴ and R⁴′ are each alkyl. In some embodiments, R⁴ and R⁴′ are each methyl. In some embodiments, and R⁴′ taken together with the carbon atom to which they are attached form a C₃₋₆ cycloalkyl. In some embodiments, R⁴ and R⁴′ taken together with the carbon atom to which they are attached form a cyclopropyl.

In some embodiments of Formula (II), each optionally substituted alkyl is independently an optionally substituted C₁₋₆ alkyl. In further embodiments, the C₁₋₆ alkyl is selected from the group consisting of methyl, ethyl, n-propyl, isopropyl, n-butyl, isobutyl, tert-butyl, amyl, and, isoamyl. In some embodiments, the C₁₋₆ alkyl is Me or Et. In some embodiments, the C₁₋₆ alkyl is a C₁₋₆ haloalkyl. In further embodiments, the C₁₋₆ haloalkyl is selected from the group consisting of —CF₃, —CHF₂, CH₂F, and —CHBr₂. In some embodiments, the C₁₋₆ haloalkyl is CF₃. In some embodiments, the C₁₋₆ haloalkyl is CHF₂.

In some embodiments of Formula (II), each optionally substituted carbocyclyl is independently an optionally substituted C₃₋₁₂ cycloalkyl. In some embodiments, the carbocyclyl is a C₃₋₆ cycloalkyl. In some embodiments, the cycloalkyl is selected from the group consisting of cyclopropyl, cyclobutyl, cyclopentyl and cyclohexyl.

In some embodiments of Formula (II), each optionally substituted heterocyclyl is independently an optionally substituted 3-12 membered heterocycloalkyl having 1 or 2 heteroatoms independently selected from N, O, and S. In some embodiments, each optionally substituted heterocyclyl is independently an optionally substituted 3-6 membered heterocycloalkyl having 1 or 2 heteroatoms independently selected from N, O, and S. In further embodiments, the heterocycloalkyl is an optionally substituted 5-membered or 6-membered heterocycle having 1 or 2 heteroatoms independently selected from N, O, and S. In some embodiments, the heterocyclyl is selected from the group consisting of aziridinyl, azetidinyl, pyrrolidinyl, piperidinyl, morpholinyl, and thiomorpholinyl.

In some embodiments of Formula (H), each optionally substituted aryl is independently a C₆₋₁₂ aryl. In further embodiments, the C₆₋₁₂ aryl is an optionally substituted phenyl.

in some embodiments of Formula (II), each optionally substituted heteroaryl is independently a 5-12 membered heteroaryl having 1, 2, or 3 heteroatoms independently selected from N, O, and S. In some embodiments, each optionally substituted heteroaryl is independently a 5-12 membered heteroaryl having 3 heteroatoms independently selected from N, O, and S. In some embodiments, each optionally substituted heteroaryl is independently a 5-12 membered heteroaryl having 2 heteroatoms independently selected from N, O, and S. In some embodiments, each optionally substituted heteroaryl is independently a 5-12 membered heteroaryl having 1 heteroatom independently selected from N, O, and S. In further embodiments, each optionally substituted heteroaryl is an optionally substituted 5-membered or 6-membered heteroaryl having 1 heteroatom independently from N, O, and S. In some embodiments, each heteroaryl is independently selected from the group consisting of tetrazole, oxadiazole, thiadiazole, imidazole, pyrazole, thiazole, or oxazole, each of which is optionally substituted. In some embodiments, the heteroaryl is tetrazole. In some embodiments, the heteroaryl is oxadiazole.

In some embodiments, the present disclosure provides a compound selected from the group consisting of:

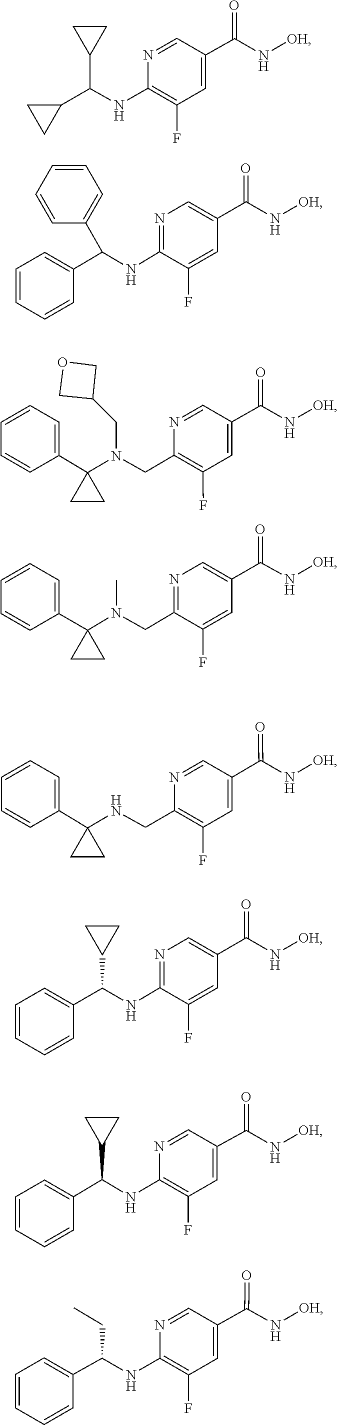

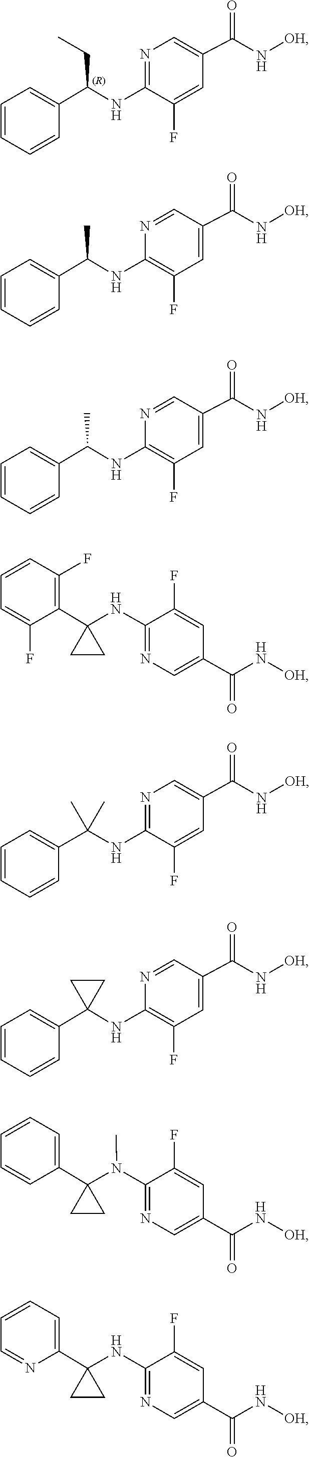

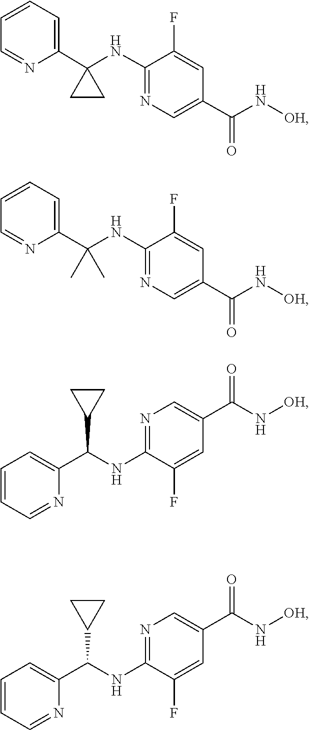

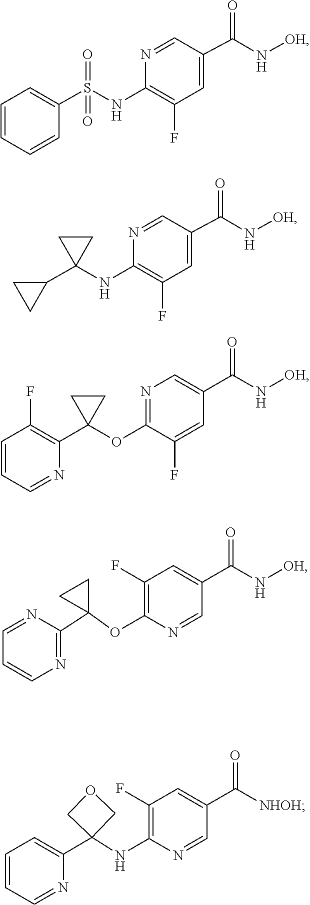

or a pharmaceutically acceptable salt thereof.

In some embodiments, the present disclosure provides a compound of Formula (IIa) or pharmaceutically acceptable salt thereof:

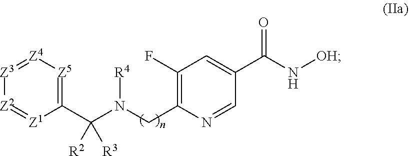

wherein:

n, R², R⁴, and R⁴ are as defined above in Formula (II); and

Z₁, Z₂, Z₃, Z₄, and Z⁵ are independently selected from N and CR⁵;

wherein R⁵ is independently selected from the group consisting of H, halogen, alkyl, haloalkyl, carbocyclyl, heterocyclyl, aryl, heteroaryl, —CO₂H, —CO₂-alkyl, —O-alkyl, haloalkyl, —O-aryl, —O-heteroaryl, —SO₂-alkyl, and —CN.

In some embodiments of Formula (IIa), Z¹, Z², Z³, Z⁴ and Z⁵ are CR⁵. In some embodiments, Z¹ is N and Z², Z³, Z⁴ and Z⁵ are CR⁵. In some embodiments, Z² is N and Z¹, Z³, Z⁴ and Z⁵ are CR⁵. In some embodiments, Z³ is N and Z¹, Z², Z⁴ and Z⁵ are CR⁵. In some embodiments, one of Z¹, Z², Z³, Z⁴ and Z⁵ is N. In some embodiments, two of Z¹, Z², Z³, Z⁴ and Z⁵ are N. In some embodiments, Z¹ and Z⁵ are each N.

In some embodiments of Formula (IIa), R⁵ is independently selected from H, halogen, alkyl, alkoxy, and haloalkyl. In some embodiments, R⁵ is independently selected from H and halogen. In some embodiments, R⁵ is independently selected from H and fluoro.

In some embodiments, the present disclosure provides a compound of Formula (IIb) or pharmaceutically acceptable salt thereof:

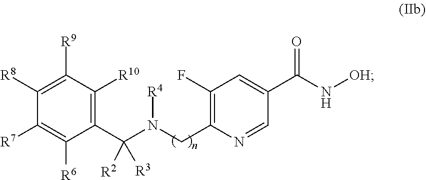

wherein:

n, R², R³, and R⁴ are as defined above in Formula (II); and

R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.

In some embodiments of Formula (IIb), R⁶, R⁷, R⁸, R⁹, and R¹⁰ are independently selected from the group consisting of H, halogen, alkyl, haloalkyl, —CO₂H, CO₂-alkyl, —O-alkyl, —O-haloalkyl, —O-aryl, —O-heteroaryl, —SO₂-alkyl, and —CN. In some embodiments, R⁶, R⁷, R⁸, R⁹, and R¹⁰ are independently selected from the group consisting of H, halogen, alkyl, haloalkyl, —O-alkyl, and —O-haloalkyl. In some embodiments, R⁶, R⁷, R⁸, R⁹, and R¹⁰ are independently selected from the group consisting of and halogen. In some embodiments, R⁶ and R¹⁰ are halogen and R⁷, R⁸, and R⁹ are H. In some embodiments, R⁶ and R¹⁰ are fluoro and R⁷, R⁸, and R⁹ are H.

In some embodiments, the present disclosure provides a compound of Formula (IIc) or pharmaceutically acceptable salt thereof:

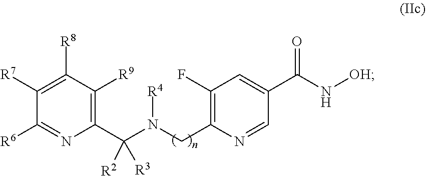

wherein:

n, R², R³, and R⁴ are as defined above in Formula (II); and

R⁶, R⁷, R⁸, and R⁹ are independently selected from the group consisting of H, halogen, alkyl, haloalkyl, carbocyclyl, heterocyclyl, aryl, heteroaryl, —CO₂H, —CO₂-alkyl, —O-alkyl, —O— haloalkyl, —O-heteroaryl, —SO₂-alkyl, and —CN.

In some embodiments of Formula (IIc), R⁶, R⁷, R⁸, and R⁹ are independently, selected from the group consisting of H, halogen, alkyl, haloalkyl, —CO₂H, —CO₂-alkyl, —O-haloalkyl, —O-aryl, —O-heteroaryl, —SO₂-alkyl, and —CN. In some embodiments, R⁶, R⁷, R⁸, and R⁹ are independently selected from the group consisting of H, halogen; alkyl, haloalkyl, —O-alkyl, and —O-haloalkyl. In some embodiments, R⁶, R⁷, R⁸, and R⁹ are independently selected from the group consisting of H and halogen. In some embodiments, R⁶ is halogen and R⁷, R⁸, and R⁹ are H. In some embodiments, R⁶ is fluoro and R⁷, R⁸, and R⁹ are H.

In another aspect, the present disclosure provides a compound of Formula (III) or pharmaceutically acceptable salt thereof:

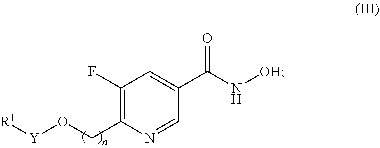

wherein:

n is 0 or 1;

Y is a bond or CR²R³;

R¹ is selected from the group consisting of H, carbocyclyl, heterocyclyl, aryl, and heteroaryl;

R² and —R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂) aryl, and (CH₂) heteroaryl; or

R¹ and R² when present taken together with the carbon atom to which they are attached form a carbocyclyl or heterocyclyl; or

R² and R³ taken together with the carbon atom to which they are attached form a carbocyclyl or heterocyclyl; and

wherein each alkyl, carbocyclyl, heterocyclyl, aryl, and heteroaryl is independently optionally substituted with one or more substituents selected from the group consisting, of halogen, oxo, hydroxy, alkoxy, —OCH₃, —CO₂CH₃, and —CH₃.

In some embodiments of Formula (III), n is 1. In some embodiments, n is 0.

In some embodiments of Formula (III), Y is a bond or CR²R³. In some embodiments, Y is S(O)₂ or CR²R³. In some embodiments, Y is a bond. In some embodiments, Y is CR²R³. In some embodiments, some embodiments, Y is S(O)₂.

In some embodiments of Formula (III), R¹ is selected from the group consisting of amido, carbocyclyl, heterocyclyl, aryl, and heteroaryl. In some embodiments, R¹ is selected from the group consisting of carbocyclyl, heterocyclyl, aryl, and heteroaryl. In some embodiments, R¹ is a heteroaryl selected from the group consisting of pyrimidinyl, pyridinyl, pyridazine, and pyrazine. In some embodiments, R¹ is pyridinyl. In some embodiments, R¹ is phenyl.

In some embodiments of Formula (III), Y is CR²R³ and R¹ is aryl or heteroaryl. In some embodiments, Y is CR²R³ and R¹ is aryl. In some embodiments, Y is CR²R³ and R¹ is heteroaryl. In some embodiments, n is 0. In some embodiments, n is 1.

In some embodiments of Formula (III), Y is a bond and R¹ is H. In some embodiments, Y is a bond and R¹ is H. In some embodiments, Y is CR²R³ and R¹ is H. In some embodiments, n is 0. In some embodiments, n is 1.

In some embodiments of Formula (III), R¹ and R² taken together with the carbon atom to which they are attached form a C₃₋₁₂ carbocyclyl. In some embodiments, the C₃₋₁₂ carbocyclyl is a propyl ring. In some embodiments, the C₃₋₁₂ carbocyclyl is a cyclobutyl ring. In another embodiment, the C₃₋₁₂ carbocyclyl is an indane ring.

In some embodiments of Formula (III), R² and R³ are independently selected from the group consisting of H, F, C₁₋₆ alkyl, C₃₋₆ cycloalkyl, —(CH₂)—C₃₋₆ cycloalkyl, 4- to 6-membered heterocyclyl, and —(CH₂)-(4- to 6-membered heterocyclyl). In some embodiments, R² and R³ taken together with the carbon atom to which they are attached form a C₃₋₆ cycloalkyl. In some embodiments, R² and R³ taken together with the carbon atom to which they are attached form a cyclopropyl, cyclobutyl, or cyclohexyl. In some embodiments, R² and R³ taken together with the carbon atom to which they are attached form a cyclopropyl. In some embodiments, R² and R³ taken together with the carbon atom to which they are attached form a 4- to 6-membered heterocyclyl. In some embodiments, R² and R³ taken together with the carbon atom to which they are attached form a azetidinyl, pyrrolidinyl, pipetidinyl, piperazinyl, morpholinyl, thiomorpholinyl, tetrahydrofuranyl, or tetrahydropyranyl.

In some embodiments of Formula (III), R² is H and R³ is C₃₋₆ cycloalkyl. In some embodiments, R² is H and R³ is cyclopropyl. In some embodiments, R² and R³ are C₁₋₆ alkyl. In some embodiments, R² and R³ are methyl.

In some embodiments of Formula (III), R¹ is C₃₋₆ cycloalkyl or aryl, R² is H, and R³ is C₃₋₆ cycloalkyl or aryl. In some embodiments, R¹ is aryl, R² is and R³ is C₃₋₆ cycloalkyl or aryl. In some embodiments, R¹ is aryl, R² is H, and R³ is C₃₋₆ cycloalkyl or C₁₋₅ alkyl. In some embodiments, R¹ is aryl, R² is H, and R³ is C₃₋₆ cycloalkyl. In some embodiments. R¹ is C₃₋₆ cycloalkyl, R² is H, and R³ is C₃₋₆ cycloalkyl or aryl. In some embodiments, R¹ is C₃₋₆ cycloalkyl, R² is and R³ is C₃₋₆ cycloalkyl or C₁₋₅ alkyl. In some embodiments, R¹ is C₃₋₆ cycloalkyl, R² is H and R³ is C₃₋₆ cycloalkyl. In some embodiments, the aryl is phenyl and C₃₋₆ cycloalkyl is cyclopropyl.

In some embodiments of Formula (III), each optionally substituted alkyl is independently an optionally substituted C₁₋₆ alkyl. In further embodiments, the C₁₋₆ alkyl is selected from the group consisting of methyl, ethyl, n-propyl, isopropyl, n-butyl, isobutyl, tert-butyl, amyl, and isoamyl. In some embodiments, the C₁₋₆ alkyl is Me or Et. In some embodiments, the C₁₋₆ alkyl is a C₁₋₆ haloalkyl. In further embodiments, the C₁₋₆ haloalkyl is selected from the group consisting of —CF₃, —CHF₂, —CH₂F, and —CHBr₂. In some embodiments, the C₁₋₆ haloalkyl is CF₃. In some specific embodiments, the C₁₋₆ haloalkyl is CHF₂.

In some embodiments of Formula (III), each optionally substituted carbocyclyl is independently an optionally substituted C₃-1? cycloalkyl. In some embodiments, the carbocyclyl is a C₃₋₆ cycloalkyl. In some embodiments, the cycloalkyl is selected from the group consisting of cyclopropyl, cyclobutyl, cyclopentyl, and cyclohexyl.

In some embodiments of Formula (III), each optionally substituted heterocyclyl is independently an optionally substituted 3-12 membered heterocycloalkyl having 1 or 2 heteroatoms independently selected from N, O, and S. In some embodiments, each optionally, substituted heterocyclyl is independently an optionally substituted 3-6 membered heterocycloalkyl having 1 or 2 heteroatoms independently selected from N, O, and S. In further embodiments, the heterocycloalkyl is an optionally substituted 5-membered or 6-membered heterocycle having 1 or 2 heteroatoms independently selected from N, O, and S. In some embodiments, the heterocyclyl is selected from the group consisting of aziridinyl, azetidinyl, pyrrolidinyl, piperidinyl, morpholinyl, and thiomorpholinyl.

in some embodiments of Formula (III), each optionally substituted aryl is independently a C₆₋₁₂ aryl. In further embodiments, the C₆₋₁₂ aryl is an optionally substituted phenyl.

In some embodiments of Formula (III), each optionally substituted heteroaryl is independently a 5-12 membered heteroaryl having 1, 2, or 3 heteroatoms independently selected from N, O, and S. In some embodiments, each optionally substituted heteroaryl is independently a 5-12 membered heteroaryl having 3 heteroatoms independently selected from —N, O, and S. In still some embodiments, each optionally substituted heteroaryl is independently a 5-12 membered heteroaryl having 2 heteroatoms independently selected from N, O, and S. In some embodiments, each optionally substituted heteroaryl is independently a 5-12 membered heteroaryl having 1 heteroatom independently selected from N, O, and S. In further embodiments, each optionally substituted heteroaryl is an optionally substituted 5-membered or 6-membered heteroaryl having 1 heteroatom independently from N, O, and S. In some embodiments, each heteroaryl is independently selected from the group consisting of tetrazole, oxadiazole, thiadiazole, imidazole, pyrazole, thiazole, or oxazole, each of which is optionally substituted. In some embodiments, the heteroaryl is tetrazole. In some embodiments, the heteroaryl is oxadiazole.

In some embodiments, the compounds of Formula (III) are selected from the group consisting of:

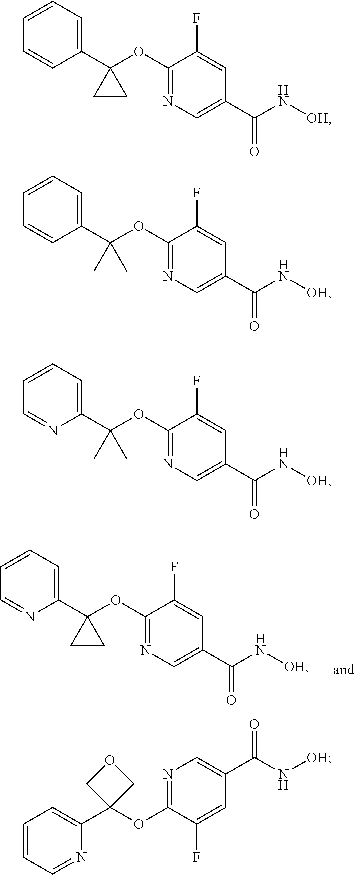

or a pharmaceutically acceptable salt thereof.

### Compounds of Formula (IV)

In some embodiments, the present disclosure provides a compound of Formula (IV) or a pharmaceutically acceptable salt thereof:

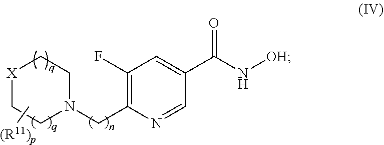

wherein:

n is 0 or 1;

p is 0, 1, 2, 3, or 4;

q is each independently 0, 1, or 2;

X is O, S(O)₂, NR¹², or CHR¹²;

R¹¹ is each independently H, F, alkyl, or oxo; or

two adjacent R¹¹ taken together with the carbon atoms to which they are attached form an aryl, heteroaryl, or heterocyclyl ring; or

two non-adjacent R¹¹ taken together with the atoms to which they are attached form a carbocyclyl or heterocyclyl ring;

R¹² is selected from the group consisting of alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R¹¹ and R¹² taken together with the carbon and/or nitrogen atoms to which they are attached form an aryl, heteroaryl ring, or heterocyclyl ring; and

wherein each alkyl, carbocyclyl, heterocyclyl, aryl, and heteroaryl is independently, optionally substituted with one or more substituents selected from the group consisting of halogen, oxo, hydroxy, alkoxy, —OCH₃, —CO₂CH₃, and —CH₃.

In some embodiments of Formula (IV), n is 1. In some embodiments, n is 0.

In some embodiments of Formula (IV), q is 2. In some embodiments of Formula (IV), q is 1. In some embodiments, q is 0.

In some embodiments of Formula (IV), X is S(O)₂, NR¹², or CHR¹². In some embodiments, X is NR¹² or CHR¹². In some embodiments, X is O. In some embodiments, X is S(O)₂. In some embodiments, X is NR¹². In some embodiments, X is CHR¹².

In some embodiments of Formula (IV), R¹¹ is oxo, alkyl, or —O-alkyl. In some embodiments, R¹¹ is oxo or alkyl. In some embodiments, R¹¹ is oxo.

In some embodiments of Formula. (IV), two adjacent RP taken together with the carbon atoms to which they are attached form an aryl or heteroaryl ring. In some embodiments, two adjacent R¹¹ taken together with the carbon atoms to which they are attached form an aryl ring. In some embodiments, the aryl ring is a phenyl ring. In some embodiments, two adjacent R¹¹ taken together with the carbon atoms to which they are attached form a heteroaryl ring or a heterocyclyl ring. In some embodiments, the heteroaryl ring or a heterocyclyl ring is a pyridinyl ring or a pyrimidinyl ring. In some embodiments, the heteroaryl ring is a pyridinyl ring.

In some embodiments of Formula (IV), two non-adjacent R¹¹ taken together with the atoms to which they are attached form a carbocyclyl or heterocyclyl ring (i.e., a bridged ring).

In some embodiments of Formula (IV), R¹² is H, alkyl, or aryl. In some embodiments, R¹² is H, Me, or Ph. In some embodiments, R¹² is Me. In some embodiments, R¹² is H. In some embodiments, R¹² is Ph.

In some embodiments of Formula (I), R¹¹ and R¹² taken together with the carbon and/or nitrogen atoms to which they are attached form an aryl, heteroaryl ring, or heterocyclyl ring. In some embodiments, aryl is phenyl. In some embodiments, heteroaryl is a 5- to 6-membered heteroatyl. In some embodiments, heterocyclyl is a 3- to 8-membered heterocyclyl having 1, 2, or 3 heteroatoms selected from the group of N, S, and O. In some embodiments, the heterocyclyl ring is

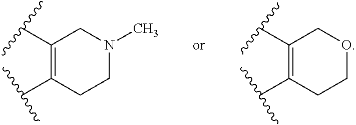

In some embodiments of Formula (IV), p is 1, 2, or, 3, or 4. In some embodiments, p is 1, 2, or 3. In some embodiments, 2, 3, or 4. In some embodiments, p is 4. In some embodiments, p is 3. In some embodiments, p is 2. In some embodiments, p is 1. In some embodiments, p is 0.

In some embodiments of Formulas (IV), when X is CHR¹², an R¹¹ and an R¹² taken together with the carbon atoms to which they are attached form an aryl ring. In some embodiments, the aryl ring is a phenyl ring.

In some embodiments of Formula (IV), when X is NR 12, an R¹¹ and an R¹² taken together with the carbon and nitrogen atoms to which they are attached form a heterocyclyl ring or a heteroaryl ring. In some embodiments, the heteroaryl ring is a pyridinyl ring.

In some embodiments of Formula, (IV), when p is 4, two adjacent R¹¹ taken together with the carbon atoms to which they are attached form an aryl ring and two adjacent R¹¹ taken together with the carbon atoms to which they are attached form a heterocyclyl ring. In some embodiments, the an ring is a phenyl ring. In some embodiments, the heterocyclyl ring is

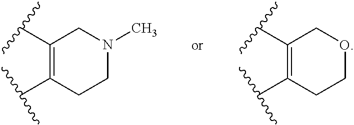

In some embodiments, n is 1 and q is 0.

In some embodiments, the compound of Formula (IV) is selected from the group consisting of:

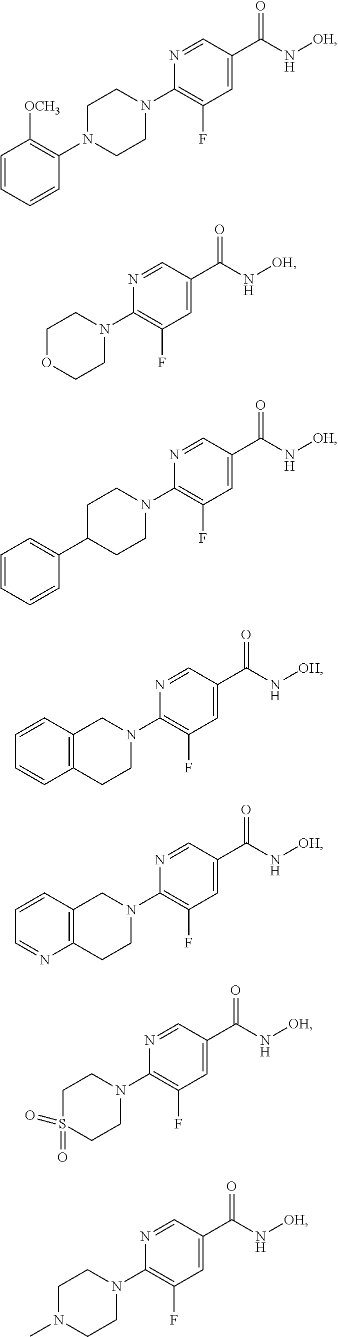

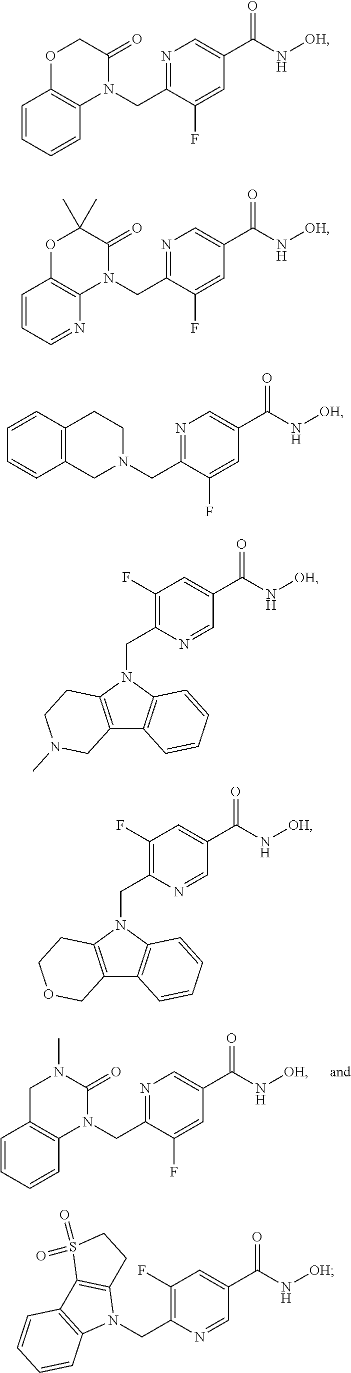

or a pharmaceutically acceptable salt thereof.

In some embodiments of Formula (IV), p is 0, 1, 2, or 3. In some embodiments, p is 0, 1, or 2. In some embodiments, p is 0 or 1. In a specific embodiment, m is p. In another specific embodiment, p is 1. In yet another specific embodiment, p is 2. In some embodiments, p is 3. In some embodiments, p is 4.

In some embodiments, the present disclosure provides a compound of Table 1.

TABLE 1
Examples of Compounds of the Present Disclosure.

| ID | Structure |
|---|---|
| 1 | 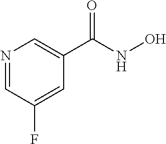 [SMILES: O=C(NO)c1cncc(F)c1] |
| 2 | 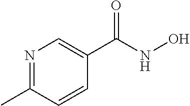 |
| I-1 | 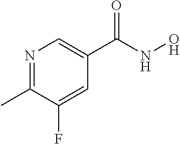 |
| I-2 | 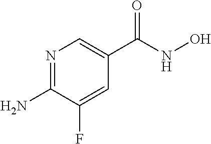 [SMILES: Nc1ncc(C(=O)NO)cc1F] |
| I-3 | 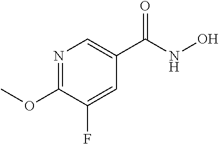 |
| I-4 | 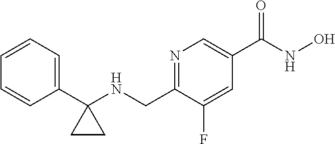 [SMILES: O=C(NO)c1cnc(CNC2(c3ccccc3)CC2)c(F)c1] |
| I-5 | 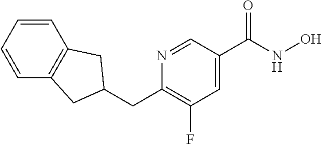 [SMILES: O=C(NO)c1cnc(CC2Cc3ccccc3C2)c(F)c1] |
| I-6 | 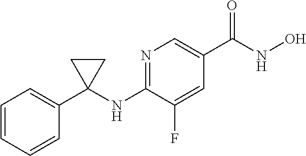 [SMILES: O=C(NO)c1cnc(NC2(c3ccccc3)CC2)c(F)c1] |
| IV-1 | 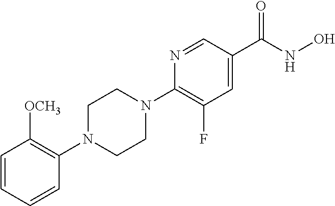 [SMILES: COc1ccccc1N1CCN(c2ncc(C(=O)NO)cc2F)CC1] |
| I-7A | 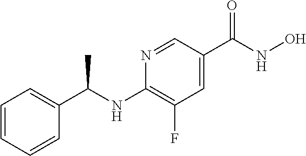 [SMILES: C[C@@H](Nc1ncc(C(=O)NO)cc1F)c1ccccc1] |
| I-7B | 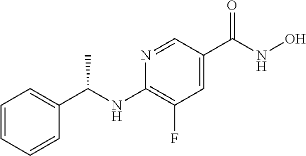 [SMILES: C[C@H](Nc1ncc(C(=O)NO)cc1F)c1ccccc1] |
| I-8B | 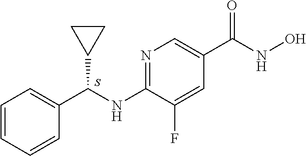 |
| I-9A | 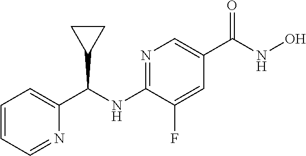 [SMILES: O=C(NO)c1cnc(N[C@@H](c2ccccn2)C2CC2)c(F)c1] |
| I-9B | 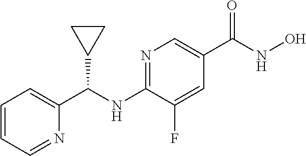 [SMILES: O=C(NO)c1cnc(N[C@H](c2ccccn2)C2CC2)c(F)c1] |
| I-10 | 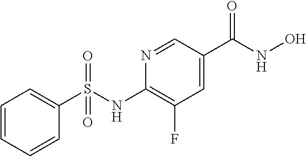 [SMILES: O=C(NO)c1cnc(NS(=O)(=O)c2ccccc2)c(F)c1] |
| IV-2 | 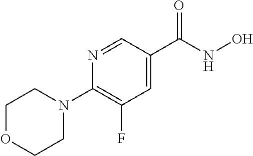 [SMILES: O=C(NO)c1cnc(N2CCOCC2)c(F)c1] |
| I-11 | 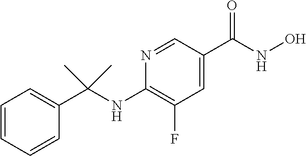 [SMILES: CC(C)(Nc1ncc(C(=O)NO)cc1F)c1ccccc1] |
| IV-3 | 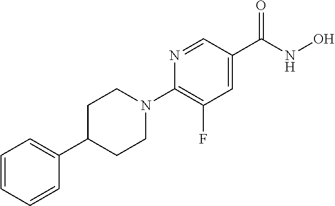 [SMILES: O=C(NO)c1cnc(N2CCC(c3ccccc3)CC2)c(F)c1] |
| IV-4 | 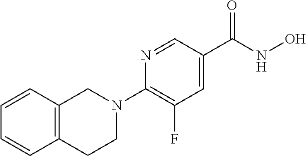 [SMILES: O=C(NO)c1cnc(N2CCc3ccccc3C2)c(F)c1] |
| I-12B | 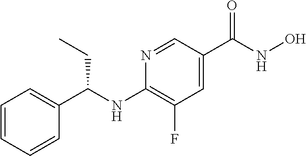 [SMILES: CC[C@H](Nc1ncc(C(=O)NO)cc1F)c1ccccc1] |
| I-8A | 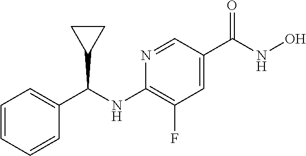 [SMILES: O=C(NO)c1cnc(N[C@@H](c2ccccc2)C2CC2)c(F)c1] |
| I-13 | 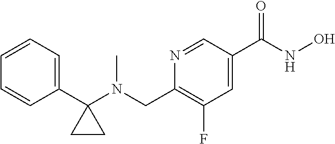 [SMILES: CN(Cc1ncc(C(=O)NO)cc1F)C1(c2ccccc2)CC1] |
| I-14 | 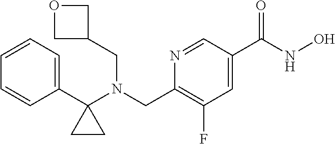 [SMILES: O=C(NO)c1cnc(CN(CC2COC2)C2(c3ccccc3)CC2)c(F)c1] |
| I-15 | 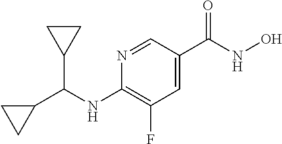 [SMILES: O=C(NO)c1cnc(NC(C2CC2)C2CC2)c(F)c1] |
| I-16 | 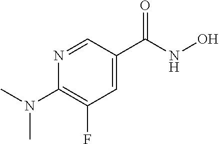 |
| IV-5 | 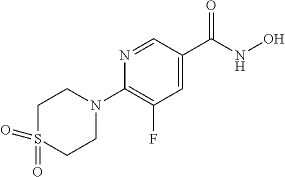 [SMILES: O=C(NO)c1cnc(N2CCS(=O)(=O)CC2)c(F)c1] |
| IV-6 | 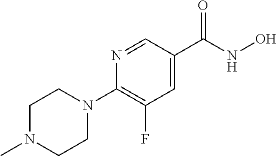 [SMILES: CN1CCN(c2ncc(C(=O)NO)cc2F)CC1] |
| 3 | 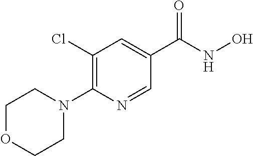 [SMILES: O=C(NO)c1cnc(N2CCOCC2)c(Cl)c1] |
| I-17 | 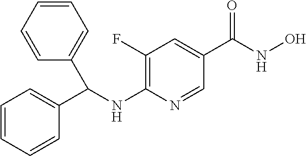 [SMILES: O=C(NO)c1cnc(NC(c2ccccc2)c2ccccc2)c(F)c1] |
| 4 | 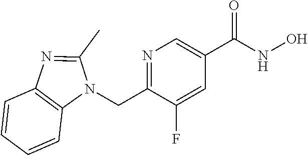 [SMILES: Cc1nc2ccccc2n1Cc1ncc(C(=O)NO)cc1F] |
| IV-7 | 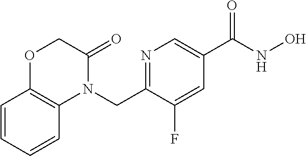 [SMILES: O=C(NO)c1cnc(CN2C(=O)COc3ccccc32)c(F)c1] |
| IV-8 | 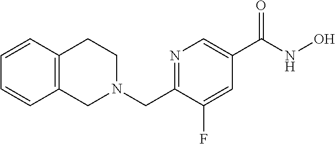 [SMILES: O=C(NO)c1cnc(CN2CCc3ccccc3C2)c(F)c1] |
| I-18 | 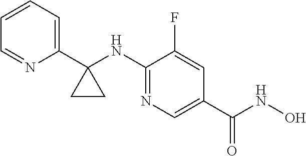 [SMILES: O=C(NO)c1cnc(NC2(c3ccccn3)CC2)c(F)c1] |
| I-19 | 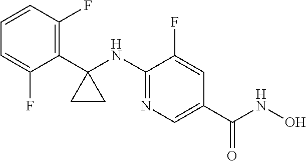 [SMILES: O=C(NO)c1cnc(NC2(c3c(F)cccc3F)CC2)c(F)c1] |
| I-20 | 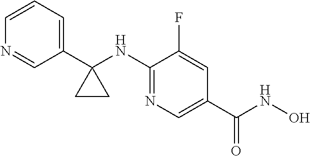 [SMILES: O=C(NO)c1cnc(NC2(c3cccnc3)CC2)c(F)c1] |
| I-21 | 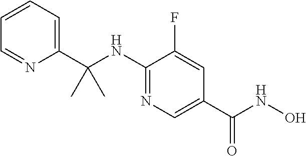 [SMILES: CC(C)(Nc1ncc(C(=O)NO)cc1F)c1ccccn1] |
| I-22 | 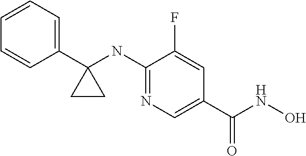 [SMILES: O=C(NO)c1cnc(NC2(c3ccccc3)CC2)c(F)c1] |
| I-23 | 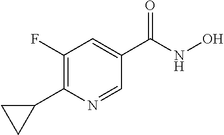 [SMILES: O=C(NO)c1cnc(C2CC2)c(F)c1] |
| I-25 | 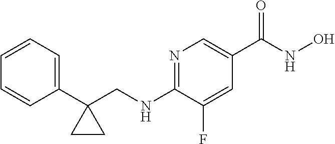 [SMILES: O=C(NO)c1cnc(NCC2(c3ccccc3)CC2)c(F)c1] |
| IV-9 | 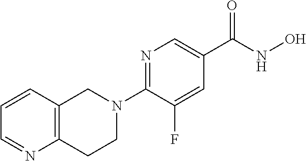 [SMILES: O=C(NO)c1cnc(N2CCc3ncccc3C2)c(F)c1] |
| III-1 | 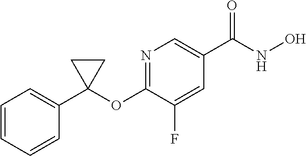 [SMILES: O=C(NO)c1cnc(OC2(c3ccccc3)CC2)c(F)c1] |
| IV-10 | 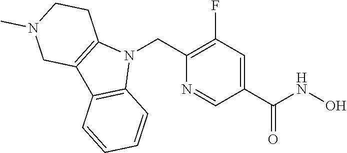 [SMILES: CN1CCc2c(c3ccccc3n2Cc2ncc(C(=O)NO)cc2F)C1] |
| 5 | 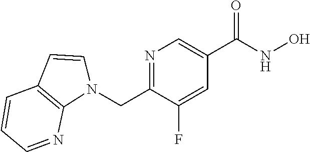 [SMILES: O=C(NO)c1cnc(Cn2ccc3cccnc32)c(F)c1] |
| I-26A | 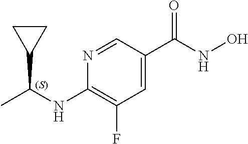 |
| I-26B | 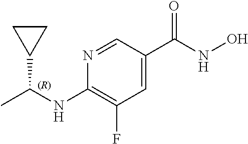 |
| 6 | 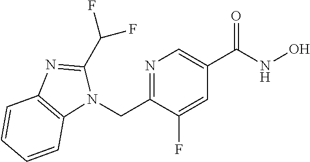 [SMILES: O=C(NO)c1cnc(Cn2c(C(F)F)nc3ccccc32)c(F)c1] |
| I-27 | 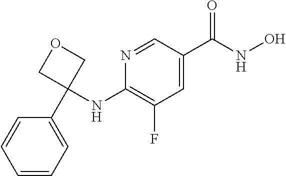 [SMILES: O=C(NO)c1cnc(NC2(c3ccccc3)COC2)c(F)c1] |
| I-28A | 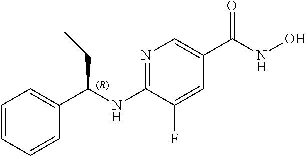 |
| I-29 | 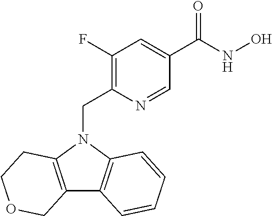 [SMILES: O=C(NO)c1cnc(Cn2c3c(c4ccccc42)COCC3)c(F)c1] |
| I-30A | 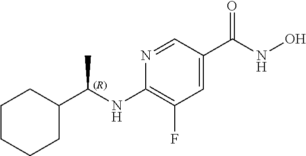 |
| I-30B | 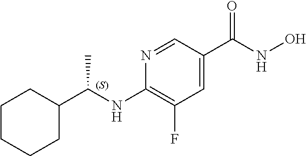 |
| I-31 | 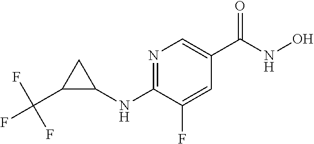 [SMILES: O=C(NO)c1cnc(NC2CC2C(F)(F)F)c(F)c1] |
| I-32 | 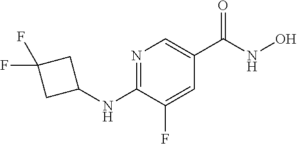 [SMILES: O=C(NO)c1cnc(NC2CC(F)(F)C2)c(F)c1] |
| I-33 | 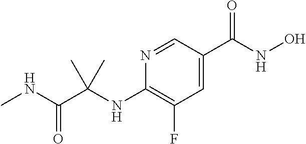 [SMILES: CNC(=O)C(C)(C)Nc1ncc(C(=O)NO)cc1F] |
| I-34 | 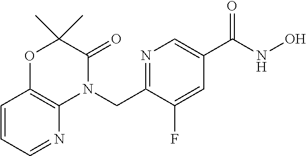 [SMILES: CC1(C)Oc2cccnc2N(Cc2ncc(C(=O)NO)cc2F)C1=O] |
| I-35 | 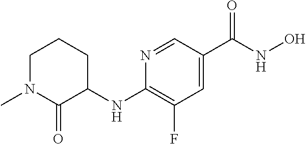 [SMILES: CN1CCCC(Nc2ncc(C(=O)NO)cc2F)C1=O] |
| I-36 | 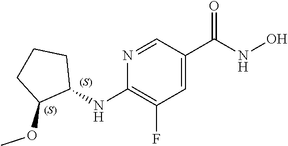 |
| I-37 | 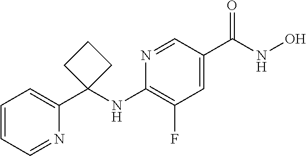 [SMILES: O=C(NO)c1cnc(NC2(c3ccccn3)CCC2)c(F)c1] |
| I-43 | 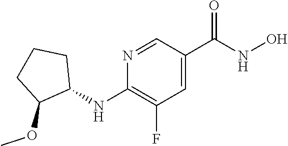 [SMILES: CO[C@H]1CCC[C@@H]1Nc1ncc(C(=O)NO)cc1F] |
| I-38 | 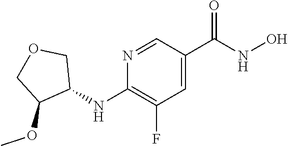 [SMILES: CO[C@H]1COC[C@@H]1Nc1ncc(C(=O)NO)cc1F] |
| I-39 | 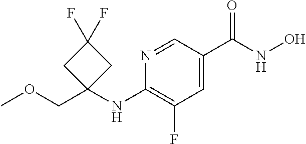 [SMILES: COCC1(Nc2ncc(C(=O)NO)cc2F)CC(F)(F)C1] |
| I-40 | 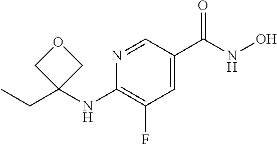 [SMILES: CCC1(Nc2ncc(C(=O)NO)cc2F)COC1] |
| I-41 | 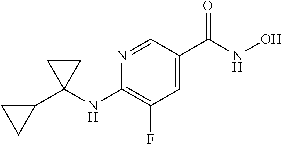 [SMILES: O=C(NO)c1cnc(NC2(C3CC3)CC2)c(F)c1] |
| I-42A | 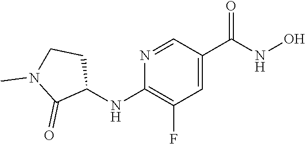 [SMILES: CN1CC[C@H](Nc2ncc(C(=O)NO)cc2F)C1=O] |
| I-43A | 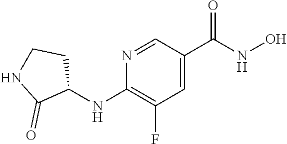 [SMILES: O=C(NO)c1cnc(N[C@H]2CCNC2=O)c(F)c1] |
| I-42B | 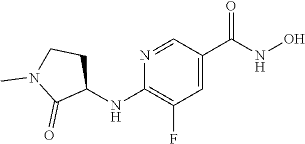 [SMILES: CN1CC[C@@H](Nc2ncc(C(=O)NO)cc2F)C1=O] |
| I-43B | 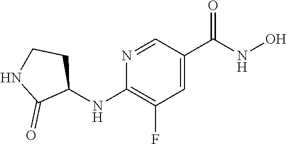 [SMILES: O=C(NO)c1cnc(N[C@@H]2CCNC2=O)c(F)c1] |
| I-44 | 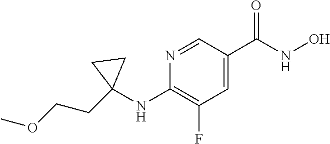 [SMILES: COCCC1(Nc2ncc(C(=O)NO)cc2F)CC1] |
| I-45 | 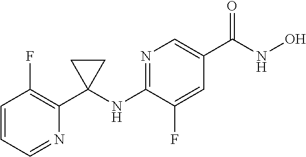 [SMILES: O=C(NO)c1cnc(NC2(c3ncccc3F)CC2)c(F)c1] |
| III-2 | 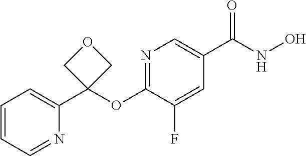 [SMILES: O=C(NO)c1cnc(OC2(c3ccccn3)COC2)c(F)c1] |
| I-46 | 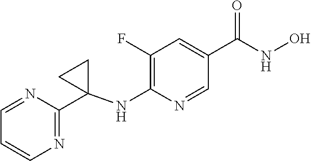 [SMILES: O=C(NO)c1cnc(NC2(c3ncccn3)CC2)c(F)c1] |
| I-47 | 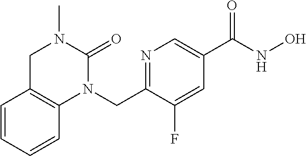 [SMILES: CN1Cc2ccccc2N(Cc2ncc(C(=O)NO)cc2F)C1=O] |
| I-48 | 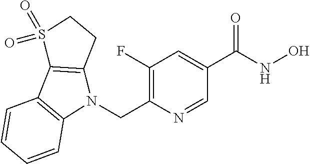 [SMILES: O=C(NO)c1cnc(Cn2c3c(c4ccccc42)S(=O)(=O)CC3)c(F)c1] |
| I-49 | 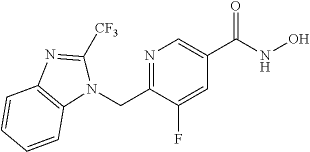 [SMILES: O=C(NO)c1cnc(Cn2c(C(F)(F)F)nc3ccccc32)c(F)c1] |
| I-50 | 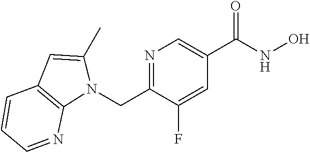 [SMILES: Cc1cc2cccnc2n1Cc1ncc(C(=O)NO)cc1F] |
| I-51 | 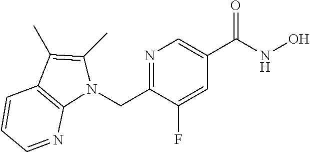 [SMILES: Cc1c(C)n(Cc2ncc(C(=O)NO)cc2F)c2ncccc12] |
| I-52 | 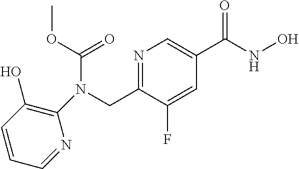 [SMILES: COC(=O)N(Cc1ncc(C(=O)NO)cc1F)c1ncccc1O] |
| I-53 | 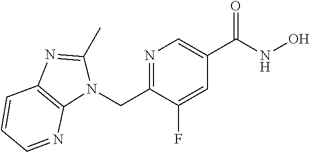 [SMILES: Cc1nc2cccnc2n1Cc1ncc(C(=O)NO)cc1F] |
| I-54 | 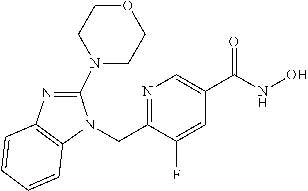 [SMILES: O=C(NO)c1cnc(Cn2c(N3CCOCC3)nc3ccccc32)c(F)c1] |
| I-55 | 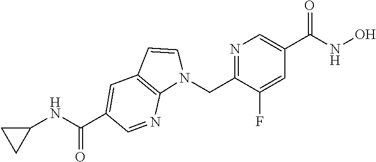 [SMILES: O=C(NO)c1cnc(Cn2ccc3cc(C(=O)NC4CC4)cnc32)c(F)c1] |
| I-56 | 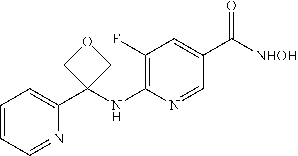 |

### Pharmaceutical Compositions

In various embodiments of the present disclosure, pharmaceutical compositions comprising one or more compounds disclosed herein, or a pharmaceutically acceptable solvate, hydrate, tautomer, N-oxide, or salt thereof, and a pharmaceutically acceptable excipient or adjuvant is provided. The pharmaceutically acceptable excipients and adjuvants are added to the composition or formulation for a variety of purposes. In some embodiments, a pharmaceutical compositions comprising one or more compounds disclosed herein, or a pharmaceutically acceptable solvate, hydrate, tautomer, NT oxide, or salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

### EXAMPLES

The invention is further illustrated by the following examples. The examples below are non-limiting and merely representative of various aspects of the invention. Solid and dotted wedges within the structures herein disclosed illustrate relative stereochemistry, with absolute stereochemistry depicted only when specifically stated or delineated.

General Methods

All reagents, for which the synthesis is not described in the experimental part, are either commercially available, or are known compounds or may be formed from known compounds by known methods by a person skilled in the art.

The compounds and intermediates produced according to the methods of the present disclosure may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using e.g. prepacked silica gel cartridges, e.g. RediSep®R_{f} and eluents such as gradients of 0-100% ethyl acetate in hexanes or 0-100% of 10% MeGH in CH₂Cl₂

Purification methods as described herein may provide compounds of the present disclosure which possess a sufficiently basic or acidic functionality in the form of a salt, such as, in the case of a compound of the present disclosure which is sufficiently basic, a trifluoroacetate or formate salt, or, in the case of a compound of the present disclosure which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present disclosure as isolated and as described herein is not necessarily the only form in which said compound can be applied to a biological assay in order to quantify the specific biological activity.

All commercially available starting materials and reagents were used as is. ¹H Nuclear magnetic resonance (NMR) spectroscopy was carried out using a Bruker Avance III instrument operating at 400 MHz using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; m, multiplet; br, broad. Preparative HPLC purification was performed by reverse phase HPLC using Agilent Technologies 1200 Infinity Series or an equivalent HPLC system such as Teledyne ISCO CombiFlash R^{f}.

Chemical names were generated using the ChemDraw naming software (Version 17.0.0.206) by PerkinElmer Informatics, Inc. In some cases, generally accepted names of commercially available reagents were used in place of names generated by the naming software.

The following abbreviations are used in the examples, while other abbreviations have their customary meaning in the art:
BOC: tert-butoxycarbonyl protecting group
DMAP: Dimethylaminopyridine
DIPEA: Diisopropylethylamine
EDCl: 1-ethyl-3-(3-dimahylaminoprapyl)carbodiimide hydrochloride
EtOH: Ethanol
EtOC(O)Cl: Ethyl chloroformate
EtOAc: Ethyl acetate
h: hour(s)
HCl: Hydrochloric acid.
HOBt: Hydroxybenzotriazole
KO^{t}Bu: Potassium t-butoxide
Liter
LCMS: liquid chromatography-mass spectrometry
M: Molar
MeOH: Methanol
min: Minute(s)
μL: Microliter
Milliliter
N: Normal
N₂: Nitrogen
n-BuLi n-butyl lithium
NMR: nuclear magnetic resonance spectroscopy
ppm: parts per million
PCC: Pyridinium chlorochromate
PPTS: Pyridinium p-toluenesulfonate
rt: loom temperature
Rt: Retention time
sat: Saturated
Sphos: 2-dicyclohexylphosphino-2′,6′-dimethoxybiphenyl
TFA: Triethylamine
THF: Tetrahydrofuran
TMS-Cl: Trimethylsilyl chloride

Analytical LC-MS Methods

Column: Eclipse Plus C18 4.6×3.5 μm; eluent A: 0.1% TFA in H₂O; eluent B: 0.1% TFA in CH₃CN; gradient: 20-100% over 4 minutes; flow: 1.5 mL/min; injection volume 1-5 μL; temperature: 23° C.; UV scan: 220 and 250 nm; signal settings—scan positive mode.

Analytical HPLC Methods

Column: Eclipse Plus C18 4.6×110 mm; eluent A: 0.1% TFA in H₂O; eluent B: 0.1% TFA in CH₃CN; gradient: 10-100% eluent B over 10 minutes; flow: 1 ml min; injection volume 1-5 μL; temperature: 23° C.; UV scan: 220, 254 and 280 nm (method 1); 20-100% eluent B over 10 minutes; flow: 1 mL/min; injection volume 1-5 μL; temperature: 23° C.; UV scan:220, 254 and 280 nm (method 2).

Preparative HPLC

Instrument: Agilent Technologies 1200 Infinity Series Column: Gemini 5 μm NX-C18 110 Å, 250×21.2 mm; eluent A: 0.1% TFA in H₂O, eluent 0.1% TTA in CH₃CN; gradient: 10-100%; flow: 20 mL/min; injection volume 0.5-2 mL; temperature: 23° C.; UV scan: 254 and 220 nm.

### Synthesis of Compounds

### Example 1

### Preparation of 5-Fluoro-N-hydroxynicotinamide (1)

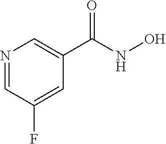 [SMILES: O=C(NO)c1cncc(F)c1]

To a cold solution of the commercially available methyl 5-fluoronicotinate (100 mg, 0.64 mmol) in methanol-THF (4 mL, 1:1) in an ice bath, was added aqueous 50% NH₂OH (1.28 g, 19.2 mmol) and KOH (362 Trig, 6.4 mmol), The reaction mixture was stirred and allowed to warm up to ambient temperature. Monitored by LCMS. After the reaction was completed, 1 N HCl was added carefully until pH 6-7 followed by addition of ethyl acetate. The organic layer was washed with water, dried (Na₂SO₄), filtered and concentrated. The resulting white solid was triturated in ethyl acetate/hexane, and filtered to afford the title compound, 50 mg (49.7%).

### Analysis of 5-fluoro-N-hydroxynicotinamide

¹H NMR (400 MHz, CD₃OD-d₄) δ 8.78 (s, 1H), 8.66 (d, J=2.69 Hz, 1H), 7.98 (br d, J=9.05 Hz, 1H), 4.64 (s, 1H).

LC-MS: tR (min) 1.21 (20-100% ACN with 0.1% TFA 4 min), m/z [M+H]⁺ C₆H₅FN₂O₂ requires: 156.0, found 157.1

HPLC tR (min) 2.70, 98% (20-100% ACN with 0.1% TFA 10 min.)

### Example 2

### Preparation of 6-methyl-N-hydroxynicotinamide (2)

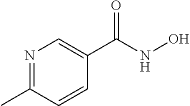

Following the same experimental procedure as described in Example 1, methyl 6-methylnicotinate was converted to the title compound by employing the commercially available methyl 6-methyl-N-nicotinate instead.

### Analysis of 6-methyl-N-hydroxynicotinamide

¹H NMR (400 MHz, DMSO-d₆) δ 11.32 (br s, 1H) 9.15 (br s, 1H) 8.79 (d, J=1.71 Hz, 1 H) 7.99 (dd, J=8.07, 2.45 Hz, 1 H) 7.35 (d, J=8.07 Hz, 1H), 3.30 (s, 3H).

LC-MS: tR (min) 1.36 (20-100% ACN with 0.1% TFA 4 min), m/z [M+H]⁺ C₇H₈N₂O₂ requires: 152.1, found 153.1

HPLC tR (min) 1.21, 100% (20-100% ACN with 0.1% TFA 10 min.)

### Example 3

### Preparation of 5-Fluoro-6-methy-N-hydroxynicotinamide (I-1)

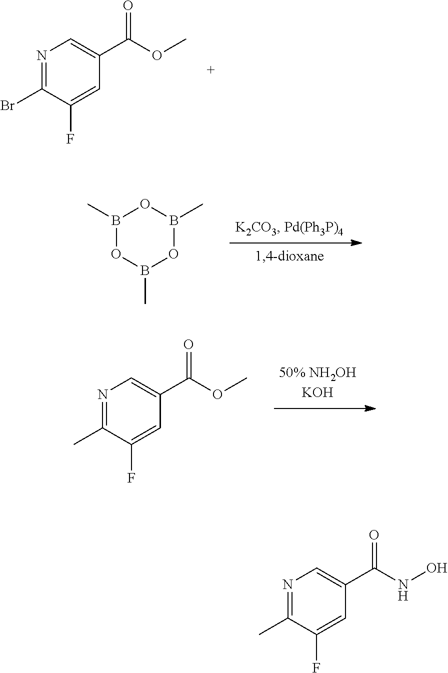

### Step 1: Methyl 5-fluoro-6-methylnicotinate

The commercially available methyl 6-promo 5-fluoronicotinate (360 mg, 1.54 mmol) was mixed with trimethyl-1,3,5,2,4,6-trioxatriborinane (966 mg, 7.69 mmol), K₂CO₃ (319 mg, 2.31 mmol), and Pd(Ph₃P)₄, (196 mg, 0.169 mmol) in 1,4-dioxane (5 mL), The mixture was sparged with N₂ and stirred at 110° C. in a sealed tube under N₂ atmosphere for 18 h. After cooled, the reaction was added ethyl acetate and washed with water. The organic layer was dried (Na₂SO₄) and filtered through Celite. Solvent was evaporated and the resulting residue was purified by chromatography (Silica gel, hexane/ethyl acetate, 1:0 to 1:1) to afford the title compound, 198 mg (76%). LC-MS: m/z [M+H]⁺ 170.1.

### Step 2: 5-Fluoro-6-methy-N-hydroxynicotinamide

Following the same experimental procedure as described in Example 1, methyl 5-fluoronicotinate (20 mg) from above Step 1 was converted to the title compound as a white solid, 8 mg (40%).

### Analysis of 5-fluoro-6-methy-N-hydroxynicotinamide

¹H NMR (400 MHz, CD₃OD-d₄) δ 8.65 (s, 1H), 7.88 (d, J=9.78 Hz, 1H), 4.64 (s, 2H), 2.57 (d, j=2.93 Hz, 3 H).

LC-MS: tR (min) 1.26 (20-100% ACN with 0.1% TFA 4 min), m/z [M+H]⁺ C₇H₇FN₂O₂ requires: 170.1, found 171.1

HPLC tR (min) 3.14, 98% (20-100% ACN with 0.1% TFA 10 min.)

### Example 4

### Preparation of 6-amino-5-fluoro-N-hydroxynicotinamide (I-2)

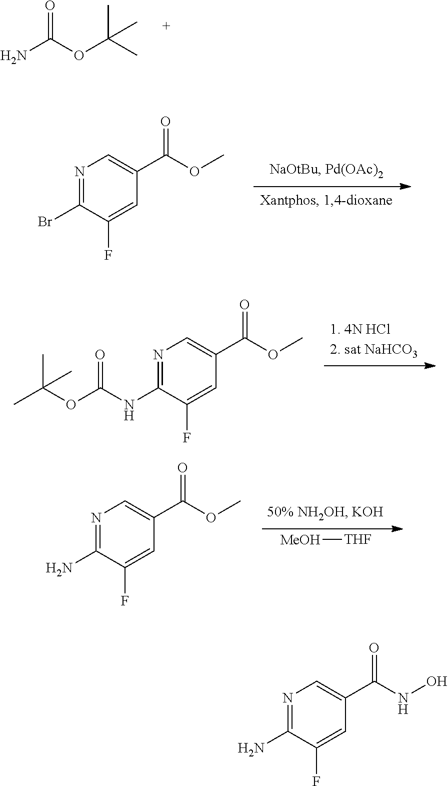

### Step 1: Tert-butyl-(3-fluoro-5-(hydroxycarbamoy)pyridine-2-yl)carbamate

Methyl 6-bromo 5-fluoronicotinate (300 mg, 1.28 mmol) was mixed with tort-butyl carbamate (150 mg, 1.28 mmol), sodium tert-butoxide (123 mg, 1.28 mmol), palladium acetate (7.20 mg, 0.032 mmol) and Xantphos (37.1 mg, 0.064 mmol) in 1,4-dioxane in an oven-dried sealed tube. After evacuated and refilled with N₂ (X3), the mixture was stirred and heated at 75° C. for 5 h. After cooled, the reaction was added ethyl acetate and washed with water. The organic layer was dried (Na₂SO₄), concentrated and purified by chromatography (Silica gel, hexane/ethyl acetate, 1:0 to 5:1) to afford a white solid as the title compound, 250 mg (72.1%). LC-MS: m/z [2M+Na]⁺ 563.5

### Step 2: Methyl 6-amino-5-fluoronicotinate hydrochloride

Tert-butyl-(3-fluoro-5-(hydroxycarbamoy)pyridine-2-yl)carbamate obtained from above Step 1 was treated with 4 N HCl in methanol (5 ml) at ambient temperature for 18 h. Solvent was evaporated and a white solid was obtained, 150 mg (65%) as the title compound. LC-MS: m/z [M+H]⁺ 171.1

### Step 3: 6-Amino-5-fluoro-N-hydroxynicotinamide

Following the same experimental procedure as described in Example 1, methyl 6-amino-5-fluoronicotinate hydrochloride from above Step 2 which was first treated with saturated NaHCO₃ and extracted with ethyl acetate/MeOH was then converted to the title compound.

### Analysis of 6-amino-5-fluoro-N-hydroxynicotinamide

¹H NMR (400 MHz, METHANOL-d₄) δ 8.21 (s, 1H) 7.66 (d, 1=9.78 Hz, 1H).

LC-MS: tR (min) 1.36 (20-100% ACN with 0.1% TFA 4 min), m/z [M+H]⁺ C₆H₆FN₃O₂ requires: 171.1, found: 172.1.

HPLC tR (min) 2.36, 96% (20-100% ACN with 0.1% TFA 10 min.)

### Example 5

### Preparation of 5-fluoro-6-methoxyl-N-hydroxynicotinamide (I-3)

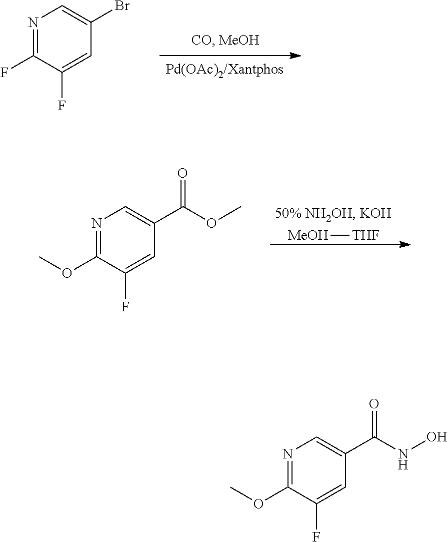

### Step 1: Methyl 5-fluoro-methoxynicotinate

The commercially available 5-bromo-2,3-difluoropyridine (520 mg, 2.7 mmol) in a vial was added palladium acetate (12 mg, 2.7 mmol), Xantphos (62 mg, 2.7 mmol), methanol (1.1 mL, 27 mmol) and triethylamine (20 mL). The reaction mixture was sparged with a CO balloon for 1 minute, sealed and heated at 65° C. under CO atmosphere. After 17 h, full consumption of the starting material was observed by TLC. The mixture was washed with water, extracted with ethyl acetate, and concentrated. The resulting residue was purified by, chromatography (Silica gel, ethyl acetate/hexane, 0-5%) to afford the title compound as a white solid, 91.7 mg (18%). LC-MS: m/z [M+H]⁺ 186.0

### Step 2: 5-Fluoro-N-hydroxy-6-methoxynicotinamide

Following the same experimental procedure as described in Example 1, methyl 6-methoxy-5-fluoronicotinate (31 mg, 0.17 mmol) from above Step 1 was converted to the title compound as a tan solid, 24.4 mg (79%).

### Analysis of 5-fluoro-N-hydroxy-6-methoxynicotinamide

¹H NMR (400 MHz, METHANOL-d4) δ 8.35 (s, 1H) 7.80 (dd, J=10.76, 1.47 Hz, 1H) 4.05 (s, 3H).

LC-MS: m/z [M+H]⁺ C₇H₇FN₂O₃ requires: 186.1, found: 187.0

HPLC tR (min) 3.92, 100% (2.0-100% ACN with 0.1% TFA 10 min.)

### Example 6

### Preparation of 5-fluoro-N-hydroxy-6-(((1-phenylcycloproyl)amino)methyl)nicotinamide (I-4)

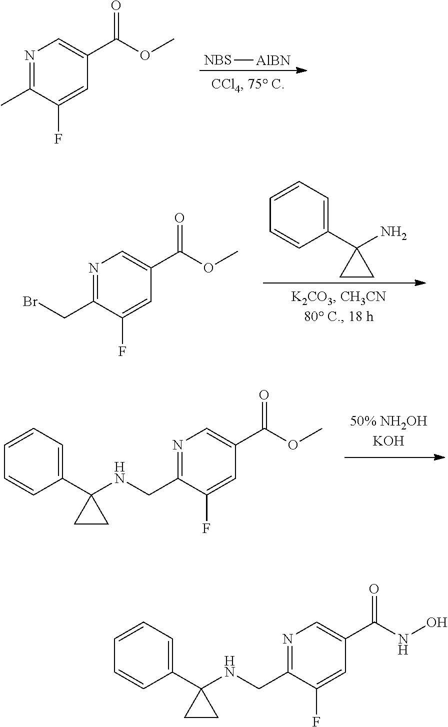

### Step 1: Methyl 6-(bromomethyl)-5-fluoronicotinate

To a solution of methyl 5-fluoro-6-methylnicotinate (195 mg, 1.15 mmol) in CCl₄ was added NBS (328 mg, 1.84 mmol) and AIBN (38 mg, 231 mmol). The reaction mixture was stirred and heated at 75° C. for 48 h. After cooled, the reaction mixture was evaporated. The resulting residue was purified by chromatography (Silica gel, hexane/ethyl acetate, 1:0 to 0:1) to afford the title compound as a white solid, 131 mg, (46%). LC-MS: m/z [M+H]⁺ 249.0

### Step 2: Methyl 5-fluoro-6-(((1-phenylcyclopropyl)amino)methy)nicotinate

Methyl 6-(bromomethyl)-5-fluoronicotinate from above Step 2 (80 mg, 0.323 mmol) was mixed with 1-phenylcyclopropan-1-amine hydrochloride (54.8 mg, 0,323 mmol) and K₂CO₃ (89.1 mg, 0.323 mmol) in acetonitrile (3 mL). The reaction was stirred and heated under a N₂ atmosphere at 77° C. for 18 h. After cooled and filtered via Celite, the mixture was concentrated. The resulting residue was purified by chromatography (Silica gel, hexane/ethyl acetate, 1:0 to 1:1) to afford the title compound as an oil, 60 mg (62%). LC-MS: m/z [M+H]⁺ 301.3

### Step 3: 5-Fluoro-N-hydroxy-6-(((1-phenylcycloproyl)amino)methyl)nicotinamide

Following the same experimental procedure as described in Example 1, methyl 5-fluoro-6-(((1-phenylcyclopropy)amino)methyl)nicatinate from above Step 2 was converted to the title compound. LC-MS: m/z [M+H]⁺ 302.3

### Analysis of 5-fluoro-N-hydroxy-6-(((1-phenylcycloproyl)amino)methyl)nicotinamide

¹H NMR (400 MHz, METHANOL-d4) δ 8.69 (s, 1H), 7.81 (d, J=10.03 Hz, 1H), 7.42 (d, J=7.83 Hz, 2H), 7.31 (t, J=7.58 Hz, 2H), 7.19-7.24 (m, 1 H), 4.64 (s, 1 H), 3.94 (s, 2 H), 1.05-1.11 (m, 2H), 0.96-1.04 (m, 2H).

LC-MS: tR (min) 1.94 (20-400% ACN with 0.1% TFA 4 min), m/z [M+H]⁺ C₁₆H₁₆FN₃O₂ requires: 301.3, found: 302.3

HPLC tR (min) 4.16, 99% (20-100% ACN with 0.1% TFA 10 min.)

### Example 7

### Preparation of 6-((2,3-dihydro-1H-inden-2-yl)amino)-5-fluoro-N-hydroxynicotinamide (I-5)

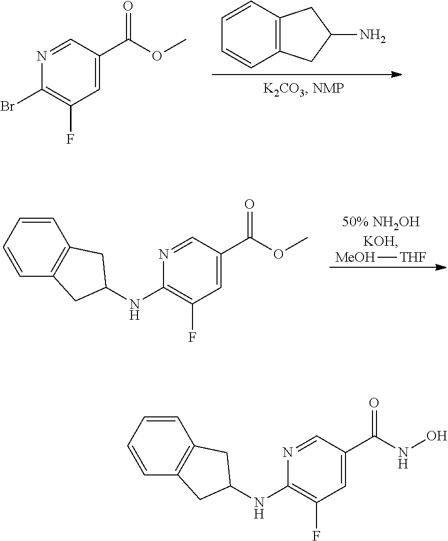

### Step 1: Methyl 6-((2,3-dihydro-1H-inden-2-yl)amino-5-fluoronicotinate

To a solution of methyl 5-fluoro-6-bromonicotinate (100 mg, 0.47 mmol) in NMP (2 mL) was added 2,3-dihydro-1H-inden-2-amine (62.6 mg, 0.47 mmol) and K₂CO₃ (195 mg, 1.41 mmol) in a vial. The reaction mixture was stirred and heated at 120° C. under N₂ for 18 h. After cooled and diluted with ethyl acetate, the organic layer was washed with water, Dried (Na₂SO₄), filtered and concentrated, the resulting residue was purified by chromatography (Silica gel, hexane/ethyl acetate, 1:0 to 0:1) to afford the title compound as a brown oil, 38 mg (31%). LC-MS: m/z [M+H]⁺ 287.3

### Step 2: 6-((2,3-Dihydro-1H-inden-2-yl)amino)-5-fluoro-N-hydroxynicotinamide

Following the same experimental procedure as described in Example 1, methyl 6-((2,3-dihydro-1H-inden-2-yl)amino-5-fluoronicotinate from above Step 1 was converted to the title compound.

### Analysis of 6-((2,3-dihydro-1H-inden-2-yl)amino)-5-fluoro-N-hydroxynicotinamide

¹H NMR (400 MHz, DMSO-d6) δ 11.0 (br, s, 1H), 8.98 (s, 1H), 8.32 (s, 1H), 7.63 (d, J=12.2 Hz, 1 H), 7.45 (br, d, J=6.60 Hz, 1H), 7.26-7.19 (m, 2 H), 7.18-7.11 (m, 2H), 4.84-4.75 (m, 1H), 3.27 (dd, J=15.9, 7.58 HZ, 2H), 2.96 (dd, J=15.9, 7.09 Hz, 2H).

LC-MS: m/z [M+H]⁺ C₁₅H₁₃FN₃O₂ requires: 287.3, found: 288.3

HPLC tR (min) 5.16, 99% (20-100% ACN with 0.1% TFA 10 min.)

### Example 8

### Preparation of 5-fluoro-N-hydroxy-6-((1-phenylcyclopropyl)amino)nicotinamide (I-6)

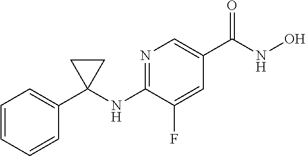 [SMILES: O=C(NO)c1cnc(NC2(c3ccccc3)CC2)c(F)c1]

### Step 1: Methyl 5-fluoro-6-((1-phenylcyclopropyl)amino)nicotinate

The title compound was synthesized by following the same experimental procedure as described in Step 1 of Example 7 employing methyl 5-fluoro-6-bromonicotinate and 1-phenylcyclopropan-1-amine hydrochloride instead. LC-MS: m/z [M+H]⁺ 287.3

### Step 2: 5-Fluoro-N-hydroxy-6-((1-phenylcyclopropyl)amino)nicotinamide

Following the same experimental procedure as describe in Example 1, methyl 5-fluoro-6-((1-phenylcyclopropyl)amino)nicotinate from above Step 1 was converted to the title compound as a solid.

### Analysis of 5-Fluoro-N-hydroxy-6-((1-phenylcyclopropyl)amino)nicotinamide

¹H NMR (400 MHz, METHANOL-d4) δ 8.18 (s, 1H), 7.63 (dd, J=11.9, 1.59 Hz, 1H), 7.27-7.18 (m, 4 H), 7.15-7.11 (m 1 H), 5.03-493 (m, 1H), 1.42-1.38 (m, 2 H), 1.36-1.32 (m, 2 H).

LC-MS: tR (min) 1.90 (20-100% ACN with 0.1% TFA 4 min) m/z [M+H]⁺ C₁₅H₁₄FN₃O₂ found: 288.3

HPLC tR (min), % (20-100% ACN with 0.1% TFA 10 min.)

### Example 9

### Preparation of 5-fluoro-N-hydroxy-6-(4-(2-methoxyphenyl)piperazin-1-yl)nicotinamide (IV-1)

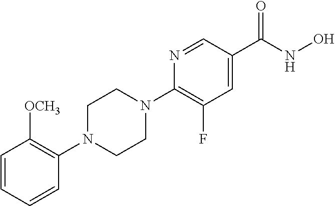 [SMILES: COc1ccccc1N1CCN(c2ncc(C(=O)NO)cc2F)CC1]

### Step 1: Methyl 5-fluoro-6-(4-(2-methyoxyphenyl)piperazin-1-yl)nicotinate

The title compound was synthesized by following the same experimental procedure as described in Step 1, Example 7 employing methyl 5-fluoro-6-bromonicotinate and 1-(2-methoxyphenyl)piperazine instead. LC-MS: m/z. [M+H]⁺ 346.3

### Step 2: 5-Fluoro-N-hydroxy-6-(4-(2-methoxyphenyl)piperazin-1-yl)nicotinamide

Following the same experimental procedure as describe in Example 1, methyl 5-fluoro-6-(4-(2-methyoxyphenyl)piperazin-1-yl)nicotinate from above Step 1 was converted to the title compound.

### Analysis of 5-fluoro-N-hydroxy-6-(4-(2-methoxyphenyl)piperazin-1-yl)nicotinamide

¹H NMR (400 MHz, METHANOL-d4) δ 8.40 (s, 1H), 7.76 (d, J=14.7 Hz, 1H), 7.01-6.87 (m, 4H), 3.81 (s, 3H), 3.67 (br, d, J=4.65 Hz, 4H), 3.12-3.12 (m, 4H).

LC-MS: m/z [M+H]⁺ C₁₇H₁₉FN₄O₂ requires: 346.4, found: 347.2

HPLC tR (min) 4.50, 98% (20-100% ACN with 0.1% TFA 10 min.)

### Example 10

### Preparation of (R)-5-fluoro-N-hydroxy-6-((1-phenylethyl)amino)nicotinamide (I-7A)

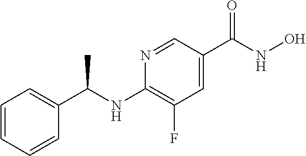 [SMILES: C[C@@H](Nc1ncc(C(=O)NO)cc1F)c1ccccc1]

### Step 1: Methyl (R)-5-fluoro-6-((1-phenylethyl)amino)nicotinate

The title compound was synthesized by following the same experimental procedure as described in Step 1 of Example 7 employing methyl 5-fluoro-6-bromonicotinate and the commercially available (R)-1-phenylethan-1-amine instead. LC-MS: m/z [M+H]⁺ 302.3

### Step 2: (R)-5-Fluoro-N-hydroxy-6-((1-phenyethyl)amino)nicotinamide

Following the same experimental procedure as described in Example 1, methyl (R)-5-fluoro-6-((1-phenylethyl)amino)nicotinate from above Step 1 was converted to the title compound.

### Analysis of (R)-5-fluoro-N-hydroxy-6-((1-phenylethyl)amino)nicotinamide

¹H NMR (400 MHz, METHANOL-d4) δ 8.19 (s, 1H), 7.57 (s, 1H), 7.57 (dd, J=11.9, 1.35 Hz, 1H), 7.39 (d, J=7.83 Hz, 2H), 7.30 (t, J=7.70 Hz, 2H), 7.23-7.18 (m, 1H), 5.34 (q, J=7.01 Hz, 1H), 1.58 (d, J=7.09 Hz, 3H).

LC-MS: tR (min) 1.86 (20-100% ACN with 0.1% TFA 4 min) m/z [M+H]⁺ C₁₄H₁₃FN₃O₂ requires: 275.3, found: 276.1

HPLC tR (min) 4.06, 99% (20-100% ACN with 0.1% TFA 10 min.)

### Example 11

### Preparation of (S)-5-fluoro-N-hydroxy-6-((1-phenylethyl)amino)nicotinamide (I-7B)

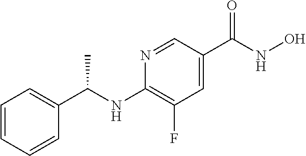 [SMILES: C[C@H](Nc1ncc(C(=O)NO)cc1F)c1ccccc1]

### Step 1: Methyl (S)-5-fluoro-6-((1-phenylethyl)amino)nicotinate

The title compound was synthesized by following the same experimental procedure as described in Step 1 of Example 7 employing methyl 5-fluoro-6-bromonicotinate and (S)-1-phenylethan-1-amine instead. LC-MS: m/z [M+H]⁺ 302.3

### Step 2: (S)-5-Fluoro-N-hydroxy-6-((1-phenylethyl)amino)nicotinamide

Following the same experimental procedure as described in Example 1, methyl (S)-5-fluoro-6-((1-phenylethyl)amino)nicotinate from above Step 1 was converted to the title compound.

### Analysis of (S)-5-fluoro-N-hydroxy-6-((1-phenylethyl)amino)nicotinamide

¹H NMR (400 MHz, METHANOL-d4) δ 8.19 (s, 1H), 7.59 (s, 1H), 7.56 (s, 1H), 7.42-7.36 (m, 2H), 7.30 (t, J=7.70 Hz, 2H), 7.23-7.18 (m, 1H), 5.34 (q, J=7.01 Hz, 1H), 1.58 (d, J=7.09 Hz, 3 H).

LC-MS: tR (min) 1.86 (20-100% ACN with 0.1% TFA 4 min), m/z [M+H]⁺ C₁₄H₁₃FN₃O₂ requires: 275.3, found: 276.1

HPLC tR (min) 4.06, 97% (20-100% ACN with 0.1% TFA 10 min.)

### Example 12

### Preparation of (S)-6-((Cyclopropyl(phenyl)methyl)amino-5-fluoro-N-hydroxynicotinamide (I-8B)

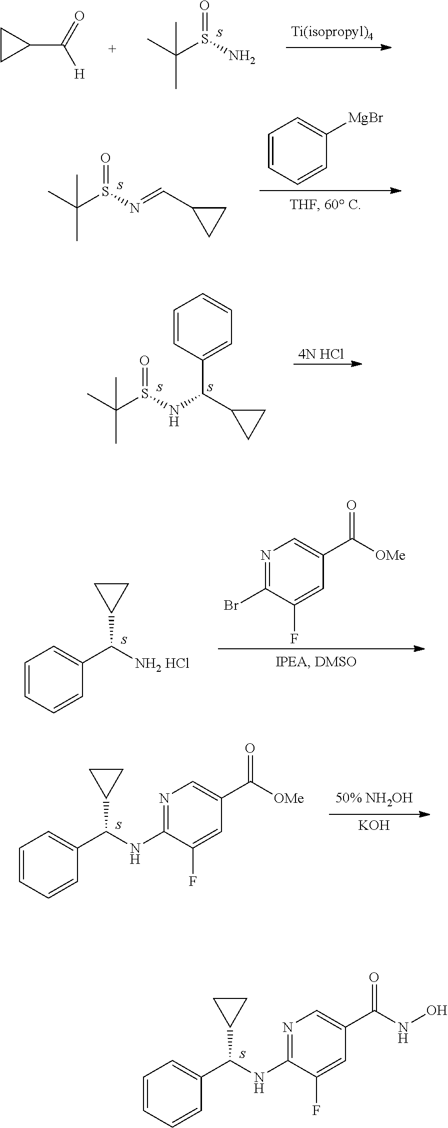

### Step 1: (S,E)-N-(Cyclopropylmethylene)-2methylpropane-sulfinamide

To a solution of cyclopropylcarboxaldehyde (5.33 mL, 71.3 mmol) in THF (150 mL) was added (S)-(−)2-methyl-2-propanesulfonamide (8.65 g, 71.3 mmol), and tetraisopropyl orthotitanate (41.8 mL, 143 mmol). The mixture was stirred at ambient temperature for 18 h. After the completion of reaction, the mixture was poured into brine and the slurry was filtered through Celite. The filtrate was extracted with ethyl acetate, and the organic layer was dried (Na₂O₄) and concentrated. The snide title compound, 12.4 g (100%) was taken to the next step without purification. LC-MS: m/z [M+H]⁺ 174.2

### Step 2: (S)—N—((S)-Cyclopropyl(phenyl)methyl)-2-methylpropane-2-sulfonamide

To a solution of (S,E)-N-(cyclopropylmethylene)-2-methylpropane-sulfinamide from above Step 1 (12.4 g, 71.6 mmol) in THF (25 mL) was added phenyl magnesium bromide (1.0 N, 71.6 mL) dropwise at ambient temperature. The mixture was then stirred at 50 to 60° C. for 2 h. After the completion of reaction, the reaction mixture was quenched with saturated ammonium chloride followed by addition of water. Filtered, the sold was washed with ethyl ester and the filtrate was extracted with ethyl acetate. The combined organic layers were washed with brine, dried (Na₂SO₄), filtered, and concentrated. The resulting residue was purified by chromatography (Silica gel, hexane/ethyl acetate) to afford the title compound, 5.0 g (28%). LC-MS: m/z [M+H]⁺ 252.3

### Step 3: (S)-Cyclopropyl(phenyl)methanamine hydrochloride

(S)—N—((S)-Cyclopropyl(phenyl)methyl)-2-methylpropane-2-sulfonamide was converted to the title compound by treating with 4 N HCl in methanol from 0° C. to ambient temperature for 18 h. Ethyl acetate was added to the mixture and a white solid was formed as the title compound (85%). LC-MS: m/z [M+H]⁺ 148.2

### Step 4: Methyl (S)-6-((cyclopropyl(phenyl)methyl)amino)-5-fluoronicotinate

The commercially available methyl 5-bromo-3-fluoronicotinate (50 mg, 0.214 mmol) was mixed with (S)-cyclopropyl(phenyl)methanamine hydrochloride (59 mg, 0.320 mmol) and diisopropylethylamine (166 mg, 1.28 mmol) in DMSO (2 mL). The mixture was stirred and heated under microwave at 100° C. for 1 h. After cooled, the reaction was added water and extracted with ethyl acetate. The organic layers were dried (Na₂SO₄), filtered and concentrated. The resulting residue was purified by chromatography (Silica gel, hexane/ethyl acetate, 1:0 to 1:1) to afford the title compound, 10 mg, (15.5%). LC-MS: m/z [M+H]⁺ 301.3

### Step 5: (S)-6-((Cyclopropyl(phenyl)methyl)amino-5-fluoro-N-hydroxynicotinamide

Following the same experimental procedure as described in Example 1, methyl (S)-6-((cyclopropyl(phenyl)methyl)amino)-5-fluoronicotinate from above Step 4 was converted to the title compound.

Analysis of (S)-6-((cyclopropyl(phenyl)methyl)amino-5-fluoro-N-hydroxynicotinamide:

¹H NMR (400 MHz, METHANOL-d4) δ 8.16 (s, 1H), 7.58 (d, J=1.71 Hz, 1H), 7.49-7.41 (m, 2H), 7.36-7.27 (m, 2H), 7.24-7.18 (m, 1H), 4.59-4.55 (m, 1H), 1.40-1.30 (m, 1 H), 0.66-0.60 (m, 2H), 0.40-0.30 (m, 2H).

LC-MS tR (min) 3.67 (20-100% ACN with 0.1% TFA 6 min), m/z [M+H]⁺ C₁₆H₁₆FN₃O₂ requires: 301.3, found: 302.1

HPLC tR (min) 4.09, 94% (20-100% ACN with 0.1% TFA 10 min.)

### Example 13

### Preparation of (R)-6-((cyclopropyl(pyridine-2-yl)methyl)amino)-5-fluoro-N-hydroxynicotinamide (I-9A)

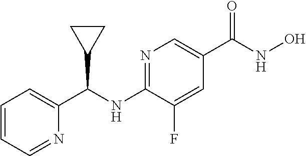 [SMILES: O=C(NO)c1cnc(N[C@@H](c2ccccn2)C2CC2)c(F)c1]

The title compound was synthesized by following the same experimental procedure as described in Example 12 employing 5-bromo-2,3-difluoropyridine and (R)-cyclopropyl(pyridine-2-yl)methanamine instead.

### Analysis of (R)-6-((cyclopropyl(pyridine-2-yl)methyl)amino)-5-fluoro-N-hydroxynicotinamide

¹H NMR (400 MHz, METHANOL-d₄) δ 8.49 (d, J=4.65 HZ, 1H), 8.14 (s, 1H), 7.80 (t, J=7.83 Hz, 1H), 7.51-7.61 (m, 2H), 7.33-7.27 (m, 1H), 4.60-4.65 (m, 1H), 0.72-0.62 (m, 1 H), 0.59-0.49 (m, 3H).

LC-MS: tR (min) 1.38 (20-100% ACN with 0.1% TFA 6 min), m/z [M+H]⁺ C₁₄H₁₆FN₄O₂ requires: 302.3, found: 303.1

HPLC tR (min) 3.38, 98% (20-100% ACN with 0.1% TFA 10 min.)

### Example 14

### Preparation of (S)-6-((cyclopropyl(pyridine-2-yl)methyl)amino)-5-fluoro-N-hydroxynicotinamide (I-9B)

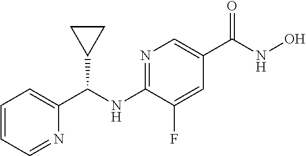 [SMILES: O=C(NO)c1cnc(N[C@H](c2ccccn2)C2CC2)c(F)c1]

The title compound was synthesized by following the same experimental procedure as described in Example 12 employing 5-bromo-2,3-difluoropyridine and (S)-cyclopropyl(pyridine-2-yl)methanamine instead.

### Analysis of (S)-6-((cyclopropyl)(pyridine-2-yl)methyl)amino)-5-fluoro-N-hydroxynicotinamide

¹H NMR (400 MHz, METHANOL-d4) δ 8.49 (d, J=4.65 HZ, 1H), 8.14 (s, 1H), 7.80 (t, J=7.83 Hz, 1H), 7.51-7.61 (m, 2H), 7.33-7.27 (m, 1H), 4.60-4.65 (m, la), 0.72-0.62 (m, 1H), 0.59-0.49 (m, 3H).

LC-MS: tR (min) 1.41 (20-100% ACN with 0.1% TFA 6 min), m/z [M+H]⁺ C₁₄H₁₆FN₄O₃ requires: 302.3, found: 303.1

HPLC tR (min) 3.38, 98% (20-100% ACN with 0.1% TFA 10 min.)

### Example 15

### Preparation of 5-fluoro-N-hydroxy-6-(phenylsulfonamido)nicotinamide (I-10)

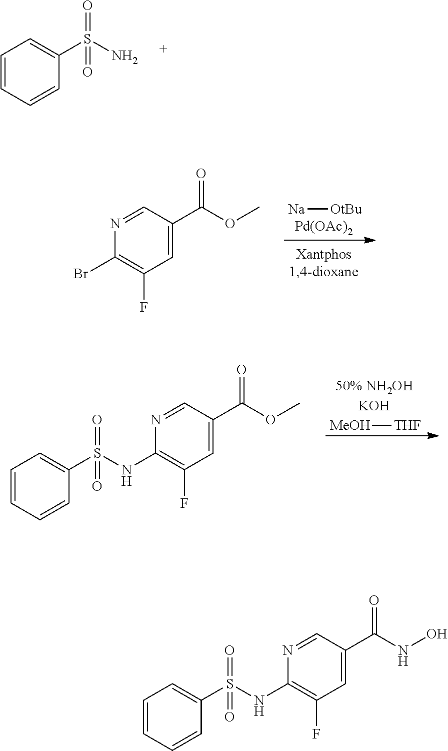

### Step 1: Methyl 5-fluoro-6-(phenylsulfonamido)nicotinate

The commercially available methyl 6-bromo 5-fluoronicotinate (100 mg, 0.427 mmol) was mixed with benzenesulfonamide (67.2 mg, 0.427 mmol), sodium tert-butoxide (41.1 mg, 0.427 mmol), palladium acetate (2.4 mg, 0.0107 mmol) and Xantphos (12.4 mg, 0.0214 mmol) in 1,4-dioxane under N₂ atmosphere in an oven-dried sealed tube, the reaction mixture was stirred and heated at 120° C. for 1 h under microwave. After cooled, the reaction was added saturated NaHCO₃, and extracted with ethyl ester (X3). The combined organic layers were dried (Na₂SO₄), filtered, and concentrated. The resulting residue was purified by chromatography (Silica gel, DCM/15% MeOH in DCM, 1:0 to 0:1) to afford the title compound as a white solid, 45 mg (34%). LC-MS: m/z [M+H]⁺ 311.3

### Step 2: 5-Fluoro-N-hydroxy-6-(phenylsulfonamido)nicotinamide

Following the same experimental procedure as described in Example 1, methyl 5-fluoro-6-(phenyl-sulfonamido)nicotinate from above Step 1 was converted to the title compound.

### Analysis of 5-fluoro-N-hydroxy-6-(phenylsulfonamido)nicotinamide

1H NMR (400 MHz, METHANOL-d4) δ 8.33 (s, 1H), 8.10 (d, J=7.34 Hz, 2H), 7.83 (s, 1H), 7.80 (s, 1H), 7.66-7.52 (m, 3H).

LC-MS: tR (min) 2.81 (20-100% ACN with 0.1% TFA 6 min), m/z [M+H]⁺ C₁₂H₁₀FSN₃O₄ requires: 311.1, found: 312.0

HPLC tR (min) 4.66, 93% (20-100% ACN with 0.1 TFA 10 min.)

### Example 16

### Preparation of 5-fluoro-N-hydroxy-6-morpholinonicotinamide (IV-2)

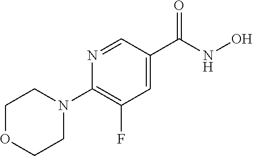 [SMILES: O=C(NO)c1cnc(N2CCOCC2)c(F)c1]

The title compound was synthesized by following the same experimental procedure as described in Example 7 employing methyl 5-fluoro-6-bromonicotinate and morpholine instead.

### Analysis of 5-fluoro-N-hydroxy-6-morpholinonicotinamide

¹H NMR (400 MHz, DMSO-d4) δ 8.35 (s, 1 H), 7.68 (d, J=14.4 Hz, 1H), 3.674-3.69 (m, 4H), 3.44-3.39 (m, 4H).

LC-MS: m/z [M+H]⁺ C₁₀H₁₂FN₃O₃ requires: 241.2, found: 242.1

HPLC tR (min) 5.56, 99% (20-100% ACN with 0.1% TFA 10 min.)

### Example 17

### Preparation of 5-fluoro-N-hydroxy-((4-phenylpropan-2-yl)amino)nicotinamide (I-11)

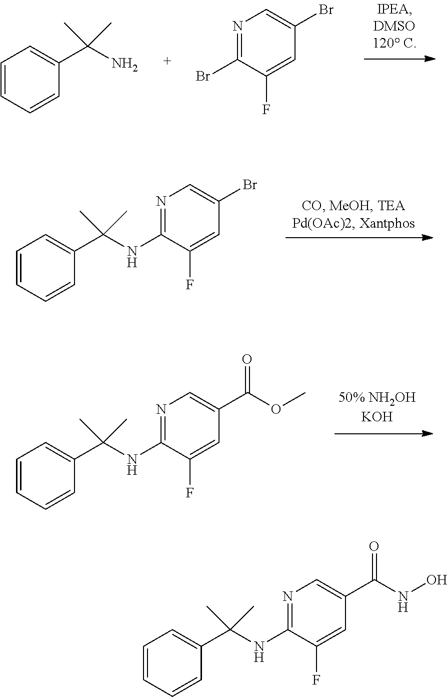

### Step 1: 5-Bromo-3-fluoro-N-(2-phenylpropan-2-yl)pyridine-2-amine

To a solution of the 5-bromo-2,3-difluoropyridine (50 mg, 0.26 mmol) in DMSO (1.0 mL) was added 2-phenylpropan-2-amine (52 mg, 0.39 mmol) and DIPEA (0.23 mL, 1.30 mmol). The mixture was stirred and heated at 120° C. for 15 h. After cooled, the reaction was added water and extracted with ethyl acetate. The combined organic layer was dried (Na₂SO₄), filtered and concentrated. The resulting residue was purified by chromatography (Silica gel, hexane/ethyl acetate, 0:1 to 9:1) to afford the title compound as a colorless oil, 25.8 mg (32.4%). LC-MS: m/z [M+H]⁺ 310.1

### Step 2: Methyl 5-fluoro-6-((2-phenylpropan-2-yl)amino)nicotinate

5-Bromo-3-fluoro-N-(2-phenylpropan-2-yl)pyridine-2-amine from above Step 1 was mixed with palladium acetate (0.8 mg, 0.0003 mmol), Xantphos (3.9 mg, 0.007 mmol), methanol and triethylamine (0.5 mL). The mixture was sparged with CO for 1 min, stirred and heated at 65° C. under CO atmosphere. Monitored by LCMS, additional palladium acetate (0.80 mg) Xantphos (3.9 mg), methanol (0.2 mL) and triethyl amine (0.2 mL) were added. After the reaction was completed, the mixture was added water/Brine and washed with ethyl acetate. The organic layer was dried (Na₂SO₄), filtered, and concentrated. The resulting residue was purified by chromatography (Silica gel, hexane/ethyl acetate, 1:0 to 4:1) to afford the title compound as a white solid, 13.5 mg (56.1%). LC-MS: m/z [M+H]⁺ 289.1

### Step 3: 5-Fluoro-N-hydroxy-6-((4-phenylpropan-2-yl)amino)nicotinamide

Following the same experimental procedure as described in Example 1, methyl 5-fluoro-6-((2-phenylpropan-2-yl)amino)nicotinate from above Step 2 was converted to the title compound.

### Analysis of 5-Fluoro-N-hydroxy-6-((4-phenylpropan-2-yl)amino)nicotinamide

¹H NMR (400 MHz, METHANOL-d4) δ 8.00 (s, 1H), 7.54 (d, J=12.2 Hz, 1H), 7.40 (d, J=8.07 Hz, 2H), 7.25 (t, J=7.58 Hz, 2H), 7.17-7.12 (m, 1H), 1.80 (s, 6H).

LC-MS: m/z [M+H]⁺ C₁₅H₁₆FN₃O₂, requires: 289.3. found, 290.1

HPLC tR (min) 5.14, 99% (20-100% ACN with 0.1% TFA 10 mm.)

### Example 18

### Preparation of 5-fluoro-N-hydroxy-6-(4-phenylpiperidin-1-yl)nicotinamide (IV-3)

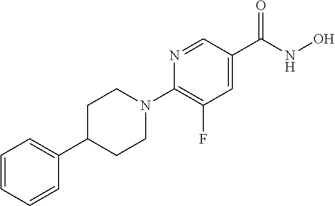 [SMILES: O=C(NO)c1cnc(N2CCC(c3ccccc3)CC2)c(F)c1]

The title compound was synthesized by following the same experimental procedure as described in Example 7 employing methyl 5-fluoro-6-bromonicotinate and 4-phenylpiperidine instead.

### Analysis of 5-fluoro-N-hydroxy-6-(4-phenylpiperidin-1-yl)nicotinamide

¹H NMR (400 MHz, DMSO-d4) δ 8.37 (s, 1H), 7.71 (d, J=14.7 Hz, 1H), 7.33-7.18 (m, 5H), 4.25 (br, d, J=13.0 Hz, 2H), 3.02 (hr, t, J=11.9 Hz, 2 H), 2.85-2.76 (m, 1H), 1.86 (br, d, J=11.4 Hz, 2H), 1.72 (qd, J=12.5, 3.30 Hz, 2H).

LC-MS: m/z [M+H]⁺ C₁₇H₁₆FN₃O₂, requires: 315.3, found: 316.1

HPLC tR (min) 5.92, 99% (20-100% ACN with 0.1% TFA 10 min.)

### Example 19

### Preparation of 6-(3,4-dihydroisoquinolin-2(1H)-yl-5-fluoro-N-hydroxynicotinamide (IV-4)

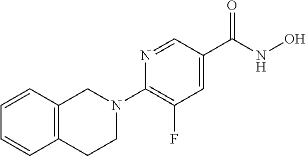 [SMILES: O=C(NO)c1cnc(N2CCc3ccccc3C2)c(F)c1]

### Step 1: Methyl 6-(3,4-dihydroisoquinolin-2(1H)-yl-5-fluoronicotinate

To a solution of methyl 6-bromo-5-fluoropyridine-3-carboxylate (0.15 g, 0.64 mmol) in Miff was added 1,2,3,4-tetrahydroisoquinoline (0.17 g, 1.28 mmol) and DMAP (2.0 mg). The mixture was stirred and heated at 80° C. under microwave for 3 h. After cooled, the reaction was added saturated ammonium chloride and extracted with ethyl acetate. The organic layer was dried (Na₂SO₄), filtered and concentrated. The resulting residue was purified by chromatography (Silica gel, hexane/ethyl acetate, 1:0 to 0:1) to afford the title compound as an oil, 160 mg (87.2%).

### Step 2: 6-(3,4-Dihydroisoquinolin-2(1H)-yl-5-fluoro-N-hydroxynicotinamide

Following the same experimental procedure as described in Example 1, methyl 5-fluoro-6-bromonicotinate and 1,2,3,4-tetrahydroisoquinoline was converted to the title compound.

### Analysis of 6-(3,4-dihydroisoquinin-2(1H)-yl-5-fluoro-N-hydroxynicotinamide

¹H NMR (400 MHz, DMSO-d4) δ 8.38 (s, 1H), 7.73 (d, J=14.7 Hz, 1H), 7.24-7.16 (m, 4 H), 4.71 (s, 2H), 3.81 (t, J=5.75 Hz, 2H), 2.93 (t, J=5.75 Hz, 2H).

LC-MS: m/z [M+H]⁺ C₁₅H₁4FN₃O₂ requires: 287.3, found: 288.1

HPLC tR, (min) 5.30, 97% (20-100% ACN with 0.1% TFA 10 min.)

### Example 20

### Preparation of (S)-6-(1-phenylpropyl)amino-5-fluoro-N-hydroxynicotinamide (I-12B)

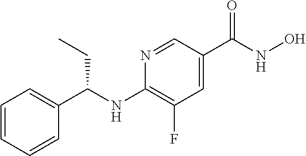 [SMILES: CC[C@H](Nc1ncc(C(=O)NO)cc1F)c1ccccc1]

The title compound was synthesized by following the same experimental procedure as described in Example 11 employing methyl 5-fluoro-6-bromonicotinate and (S)-1-phenylpropyl-1-amine instead.

### Analysis of (S)-6-(1-phenylpropyl)amino-5-fluoro-N-hydroxynicotinamide

¹H NMR (400 MHz, DMSO-d4) δ 8.19 (s, 1H), 7.56 (br, d, J=12.2 Hz, 1H), 7.41-7.36 (m, 2H), 7.3(t, J=7.58 Hz, 2H), 7.23-7.18 (m, 1H), 5.10 (t, 0.1=7.46 Hz, 1H), 2.00-1.86 (m, 2H), 1.01-0.91 (m, 3H).

LC-MS: m/z [M+H]⁺ C₁₅H₁₆FN₃O₂ requires: 289.3, found: 290.2

HPLC tR (min) 5.08, 95% (20-100% ACN with 0.1% TFA 10 min.)

### Example 21

### Preparation of (R)-6-((cyclopropyl(phenyl)methyl)amino-5-fluoro-N-hydroxynicotinamide (I-8A)

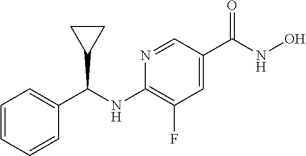 [SMILES: O=C(NO)c1cnc(N[C@@H](c2ccccc2)C2CC2)c(F)c1]

The title compound was synthesized by following the same experimental procedure as described in Example 11 employing methyl (R)-5-fluoro-6-((1-phenylethyl)amino)nicotinate instead.

### Analysis of (R)-6-((cyclopropyl(phenyl)methyl)amino-5-fluoro-N-hydroxynicotinamide

¹H NMR (400 MHz, DMSO-d4) δ 8.16 (s, 1H), 7.57 (br, d, J=12.2 Hz, 1 H), 7.44 (br, d, J=7.34 Hz, 2H), 7.30 (t, J=7.46 Hz, 2H), 7.24-7.18 (m, 1H), 4.66-4.55 (m, 2H), 1.41-1.25 (m, 2 H), 0.67-0.58 (M, 2H), 0.44 (br, s, 2H).

LC-MS: m/z [M+H]⁺ C₁₆H₁₆FN₃O₂ requires: 301.3, found: 302.1

HPLC tR (min) 4.03, 96% (20-100% ACN with 0.1% TFA 10 min.)

### Example 22

### Preparation of 6-((methyl(1-phenylcyclopropyl)amino)methyl-5-fluoro-N-hydroxynicotinamide (I-13)

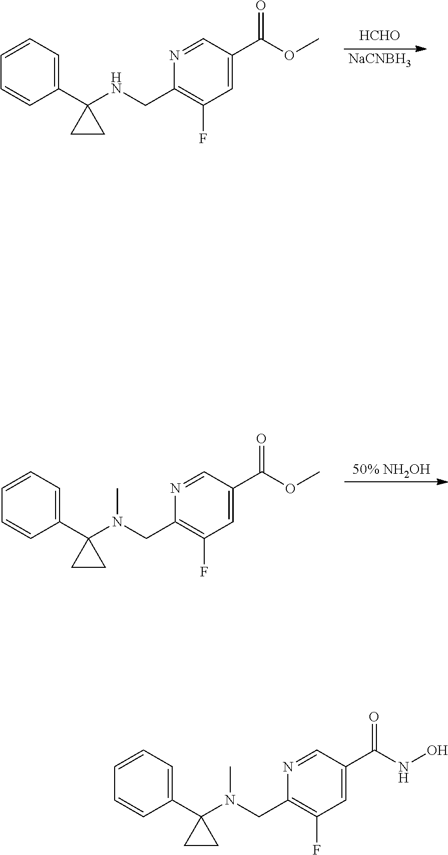

### Step 1: Methyl 5-fluoro-6-((methyl (1-phenylcyclopropyl)amino)methyl)nicotinate

Methyl 5-fluoro-6-(((1-phenylcyclopropyl)amino)methyl)nicotinate (36 mg, 0.12 mmol) was dissolved in acetonitrile/water (10:1) followed by the addition of 37% Formaldehyde (19.5 mg, 0.24 mmol), NaCNBH₃ (15.1 mg, 0.24 mmol) and 1 drop of acetic acid. The reaction mixture was stirred at ambient temperature for 15 min. Quenched with water, the mixture was extracted with ethyl acetate. The organic layer was dried (Na₂SO₄), concentrated and purified by chromatography (Silica gel, hexane/EtOAc, 1:0 to 1:1) to afford the title compound, 20 mg (53.1%). LC-MS: m/z [M+H]⁺ 315.1

### Step 2: 6-((Methyl(1-phenylcyclopropyl)amino)methy-5-fluoro-N-hydroxynicotinamide

Following the same experimental procedure as described in Example 1, methyl 5-fluoro-6-((methyl(1-phenylcyclopropyl)amino)methyl)nicotinate was converted to the title compound.

### Analysis of 6-((methyl(1-phenylcyclopropyl)amino)methyl-5-fluoro-N-hydroxynicotinamide

¹H NMR (400 MHz, DMSO-d4) δ 8.68 (s, 1 H), 7.89 (d, 0.1=10.0 1 7.47-7.29 (m, 5H), 4.54 (br, s, 1H), 3.83 (s, 2H), 2.19 (3, 3H), 1.12-1.06 (m, 2H), 0.95-0.83 (m, 2 H).

### Analysis of 6-(((Methyl(1-phenylcyclopropyl)amino)methyl-5-fluoro-N-hydroxynicotinamide

LC-MS: m/z [M+H]⁺ C₁₇H₁₈FN₃O₂ requires: 315.3, found: 316.1

HPLC tR, (min)4.12, 100% (20-100% ACN with 0.1% TFA 10 min.)

### Example 23

### Preparation of 6-(((oxetan-3-yl-methyl)(1-phenylcyclopropyl)amino)methyl)-5-fluoro-N-hydroxynicotinamide (I-14)

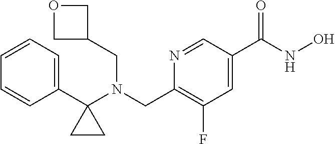 [SMILES: O=C(NO)c1cnc(CN(CC2COC2)C2(c3ccccc3)CC2)c(F)c1]

Following the same experimental procedure as described in Example above, the title compound was prepared by the reductivamination between methyl 5-fluoro-6-(((1-phenylcyclopropyl)amino)methyl)nicotinate and oxetane-3-carbaldehyde, and followed by the hydroxamic acid formation.

### Analysis of 6-(((oxetan-3-yl-methyl)(1-phenyl)amino)methyl)-5-fluoro-N-hydroxynicotinamide

¹H NMR (400 MHz, DMSO-d4) δ 8.71 (s, 1H), 7.89 (d, J=9.90 Hz, 1H), 7.49 (d, J=6.85 Hz, 2H), 7.41-7.28 (m, 3H), 4.63 (t, J=6.73 Hz, 2H), 4.54 (s, 1H), 4.15-4.09 (m, 2H), 3.96 (s, 2H), 3.23-316 (m, 1H), 2.87 (d, J=8.31, 2H), 0.78-0.82 (m, 4 H).

LC-MS: m/z [M+H]⁺ C₂₀H₂₂FN₃O₃ requires: 371.4, found: 372.1

HPLC tR (min) 4.77, 100% (20-100% ACN with 0.1% TFA 10 min.)

### Example 24

### Preparation of 6-(dicyclopropylamino)-5-fluoro-N-hydroxynicotinamide (I-45)

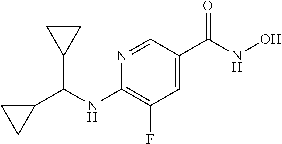 [SMILES: O=C(NO)c1cnc(NC(C2CC2)C2CC2)c(F)c1]

The title compound was prepared by following the same experimental procedure as described in Example 7 employing methyl 5-fluoro-6-bromonicotinate and 1,1-dicyclopropylmethyl amine instead.

### Analysis of 6-(dicyclopropylamino)-5-fluoro-N-hydroxynicotinamide

¹H NMR (400 MHz, DMSO-d4) δ 8.18 (s, 1H), 7.55 (d, J=11.5 Hz, 1H), 3.39-3.35 (m, 1H), 1.19-1.08 (m, 2H), 0.60-0.55 (m, 2H), 0.54-0.50 (m, 6H).

LC-MS: m/z [M+H]⁺ C₁₃H₁₆FN₃O₂ requires: 265.3, found: 266.1

HPLC tR (min) 3.37, 97% (20-100% ACN with 0.1% TFA 10 min.)

### Example 25

### Preparation of 6-(dimethylamino)-5-fluoro-N-hydroxynicotinamide (I-16)

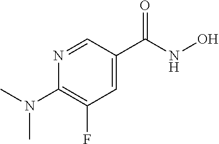

### Step 1: methyl 6-(dimethylamino)-5-fluoronicotinate

The title compound was synthesized by following the same experimental procedure as described in Step 1, Example 7 employing methyl 5-fluoro-6-bromonicotinate and dimethylamine instead.

1H NMR (400 MHz, CHLOROFORM-d) δ ppm 8.57 (t, J=1.59 Hz, 1H) 7.68 (dd, J=14.55, 1.83 Hz, 1H) 3.87 (s, 3 H) 3.22 (d, J=2.69 Hz, 6H)

LC-MS: m/z [M+H]⁺ 199.3

### Step 2: 6-(dimethylamino)-5-fluoro-N-hydroxynicotinamide

Following the same experimental procedure as describe in Example 1, methyl 6-(dimethylamino)-5-fluoronicotinate from above Step 1 was converted to the title compound.

### Analysis of 6-(dimethylamino)-5-fluoro-N-hydroxynicotinamide

1H NMR (400 MHz, DMSO-d6) δ ppm 11.05 (br s, 1H) 8.97 (s, 1H) 8.35 (s, 1H) 7.68 (br d, J=15.41 Hz, 1H) 3.09-3.14 (m, 6H)

LC-MS: m/z [M+H]⁺ C₈H₁₁FN₃O₂ requires: 200.2, found: 200.1

HPLC tR (min) 5.23, 99% (10-100% ACN with 0.1% TFA 10 min.)

### Example 26

### Preparation of 6-(1,1-dioxidothiomorpholino)-5-fluoro-N-hydroxynicotinamide (IV-5)

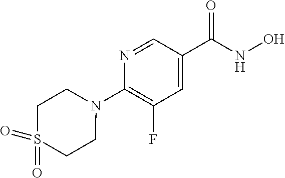 [SMILES: O=C(NO)c1cnc(N2CCS(=O)(=O)CC2)c(F)c1]

### Step 1: methyl 6-(1,1-dioxidothiomorpholino)-5-fluoronicotinate

The title compound was synthesized by following the same experimental procedure as described in Step 1, Example 7 employing methyl 5-fluoro-6-bromonicotinate and 1,1-dioxidothiomorpholine instead.

1H NMR (400 MHz, CHLOROFORM-d) δ ppm 8.63 (s, 1H) 7.85 (dd, J=13.82, 1.59 Hz, 1H) 4.20-4.28 (m, 4H) 3.91 (s, 3H) 3.07-3.22 (m, 4H)

LC-MS: m/z [M+H]⁺ 289.1

### Step 2: 6-(dimethylamino)-5-fluoro-N-hydroxynicotinamide

Following the same experimental procedure as describe in Example 1, methyl 6-(1,1-dioxidothiomorpholino)-5-fluoronicotinate from above Step 1 was converted to the title compound.

### Analysis of 6-(1,1-dioxidothiomorpholino)-5-fluoro-N-hydroxynicotinamide

1H NMR (400 MHz, DMSO-d6) δ ppm 11.20 (br s, 1H) 9.10 (br s, 1H) 8.43 (s, 1H) 7.83 (d, J=14.43 Hz, 1H) 4.04 (br s, 4H) 3.25 (br s, 4H)

LC-MS: m/z [M+H]⁺ C₁₀H₁₂FN₃O₄S requires: 290.2, found: 290.0

HPLC tR (min) 3.94, 98% (10-100% ACN with 0.1% TFA 10 mint.)

### Example 27

### Preparation of 5-fluoro-N-hydroxy-)-4- ethylpiperazin-1-yl)nicotinamide (IV-6)

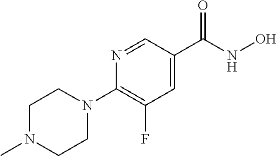 [SMILES: CN1CCN(c2ncc(C(=O)NO)cc2F)CC1]

### Step 1: methyl 5-fluoro-6-(4-methylpiperazin-1-yl)nicotinate

The title compound was synthesized by following the same experimental procedure as described in Step 1, Example 7 employing methyl 5-fluoro-6-bromonicotinate and N-methylpiperazine instead.

1H NMR (400 MHz, CHLOROFORM-d) δ ppm 8.59 (s, 1 H) 7.74 (d, J=1.22 Hz, 1H) 4.37 (s, 1 H) 3.88 (s, 3 H) 3.71-3.80 (m, 4H) 2.50-2.55 (m, 4 H) 2.34 (s, 3 H)

LC-MS: m/z [M+H]⁺ 254.3

### Step 2: 6-(dimethylamino)-5-fluoro-N-hydroxynicotinamide

Following the same experimental procedure as describe in Example 1, methyl 5-fluoro-6-(4-methylpiperazin-1-yl)nicotinate from above Step 1 was converted to the title compound.

### Analysis of 5-fluoro-N-hydroxy-6-(4-methylpiperazin-1-yl)nicotinamide

1H NMR (400 MHz, DMSO-d₆) δ ppm 11.07 (s, 1H) 9.03 (s, 1H) 8.38 (s, 1H) 7.74 (d, J=14.67 Hz, 1H) 3.51-3.58 (m, 4H) 2.40-2.45 (m, 4H) 2.21 (s, 3H)

LC-MS: m/z [M+H]⁺ C₁₁H₁₅FN₄O₂ requires: 255.3, found: 255.1

HPLC tR (min) 1.46, 99% (10-100% ACN with 0.1% TFA 10 min.)

### Example 28

### Preparation of 5-chloro-N-hydroxy-6-morpholinonicotinamide (3)

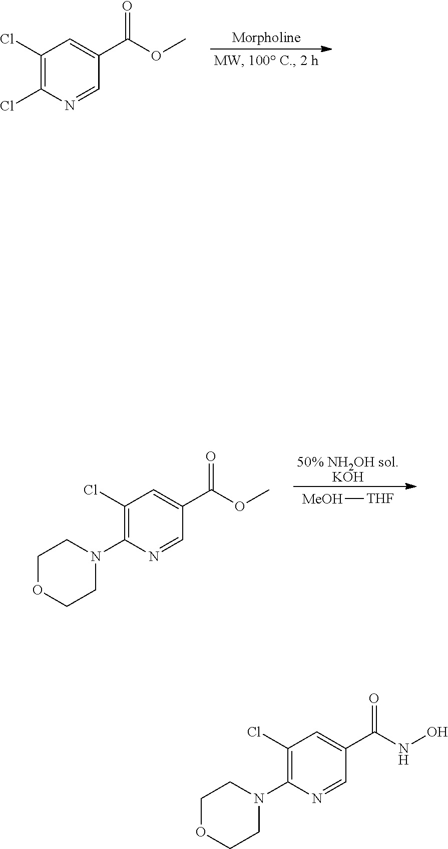

### Step 1: methyl 5-chloro-6-morpholinonicotinate

To a 10 mL microwave vial were added methyl 5,6-dichloropyridine-3-carboxylate (100 mg, 0.485 mmol) and morpholine (1 mL, 23.9 mmol). The resulting mixture was stirred at MW (100° C.) for 2 h, then cooled to rt. The crude was mixed with silica gel, the volatiles were evaporated, and then purified by ISCO using EtOAc-Hexanes (0-20%) to provide the product as a white solid (35 mg, 28%). LC-MS: m/z [M+H]⁺ 257.1.

### Step 2: 5-chloro-N-hydroxy-6-morpholinonicotinamide

Following the same experimental procedure as described in Example 1, methyl 5-chloro-6-morpholinonicotinate (35 mg, 0.14 mmol) from above Step 1 was converted to the title compound as a tan solid, 20 mg (57%).

### Analysis of 5-chloro-N-hydroxy-6-morpholinonicotinamide

¹H NMR (400 MHz, DMSO-d6) δ 11.23 (br, s, 1H), 9.12 (s, 1 H), 8.56 (m, 1 H), 8.06 (m, 1H), 3.73 (m, 4H), 3.39 (m, 4H).

LC-MS: m/z [M+H]⁺ C₁₀H₁₂FN₃O₃ requires: 257.1, found: 258.1

HPLC tR (min) 4.52, 100% (20-100% ACN with 0.1% TFA 10 min.)

### Example 29

### Preparation of 6-(benzhydrylamino)-5-fluoro-N-hydroxynicotinamide (I-17)

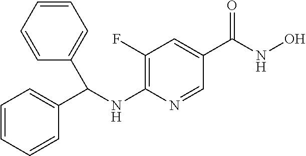 [SMILES: O=C(NO)c1cnc(NC(c2ccccc2)c2ccccc2)c(F)c1]

The title compound was synthesized by following the same experimental procedure as described in Example 12 employing 6-bromo-5-fluoropyridine-3-carboxylate and diphenylmethanamine instead.

### Analysis of 6-(benzhydrylamino)-5-fluoro-N-hydroxynicotinamide

¹H NMR (400 MHz, DMSO-d6) δ 10.97 (br, s, 1H), 8.93 (s, 1H), 8.22 (s, 1H), 7.91 (m, 1H), 87.66 (m, 1H), 3.39-7.21 (m, 11H), 6.59 (d, J=8.8 Hz, 1 H).

LC-MS: m/z [M+H]⁺ C₁₉H₁₆FN₃O₂ requires: 337.1, found: 338.1

HPLC tR (min) 5.49, 100% (20-100% ACN with 0.1% TFA 10 min.)

### Example 30

### Preparation of 5-fluoro-N-hydroxy-6-((2-methyl-1H-benzo[d]imidazol-1-yl)methyl)nicotinamide (4)

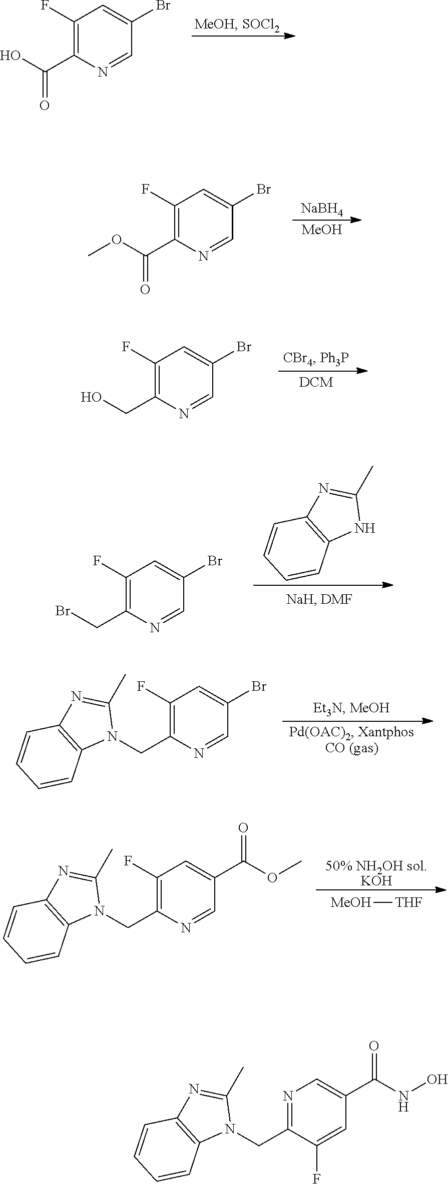

### Step 1: methyl 5-bromo-3-fluoropicolinate

To a solution of 5-bromo-3-fluoropyridine-2-carboxylic acid in MeOH (40 mL) was added SOCl₂ (2 mL) at rt slowly. The resulting mixture was stirred at 60′ C overnight. The volatiles were evaporated, the product was obtained as a white solid. (2.24 g, 100%). ¹H NMR (400 MHz, DMSO-d6) δ 8.71 (m, 1H), 8.44-8.41 (m, 1 H), 3.90 (s, 1 H); LC-MS: m/z [M+H]⁺ 233.9, 235.9.

### Step 2: (5-bromo-3-fluoropyridin-2-yl)methanol

Methyl 5-bromo-3-fluoropyridine-2-carboxylate (1.2 g; 5.13 mmol) was dissolved in dry methanol (30 mL), sodium borohydride (813 mg, 15.4 mmol) was slowly added in an ice bath. The ice bath was removed after complete addition, the mixture was warmed to room temperature and stirred overnight, LC-MS showed complete conversion; then 1 N HCl was added to adjust PH 1, again was added saturated sodium bicarbonate solution to adjust PH 10, and then extracted with ethyl acetate, the organic phase was dried over anhydrous magnesium sulfate and filtered, concentrated under reduced pressure to give the crude, which was purified by ISCO column using EtOAc-Hexanes (0-40%) to afford the product as a white solid (800 mg, 76%). ¹H NMR (400 MHz, CDCl₃) δ 8.49 (m, 1H), 7.61-7.59 (m, 1 H), 4.80-4.79 (m, 2 H) 3.51 (t, J=5.2 Hz, 1 H); LC-MS: m/z [M+H]⁺ 205.9, 207.9.

### Step 3: 5-bromo-2-(bromomethyl)-3-fluoropyridine

To a stirred solution of (5-bromo-3-fluoropyridin-2-yl)methanol (0.25 g, 1.21 mmol) and triphenyl phosphine (0.51 g, 1.94 mmol) dichloromethane (10 mL) was added, a solution of CBr₄ (0.644 g, 1.94 mmol) in DCM (5 mL) at 0° C. dropwise, and allowed to stir at room temperature overnight. The reaction mixture was quenched with sat. NaHCO₃, and extracted with dichloromethane twice. The combined organic layers were washed with brine, dried over sodium sulfate, concentrated and crude was purified by silica gel column chromatography using ethyl acetate/hexanes (0-15%) as eluent to provide the product as a white solid (240 mg, 74%). ¹H NMR (400 MHz, CDCl₃) δ 8.49 (m, 1H), 7.64-7.61 (m, 1H), 4.57 (s, 2H); LC-MS: m/z [M+H]⁺ 269.9, 271.9.

### Step 4: 1-((5-bromo-3-fluoropyridin-2-yl)methyl)-2-methyl-1H-benzo[d]imidazole

To a solution of 2-methyl-1H-1,3-benzodiazole (153 mg, 1.16 mmol) in dry DMF (3 mL) was added 60% NaH (46 mg, 1.16 mmol) at 0° C., then the mixture was stirred at 0° C. for 20 min. A solution of 5-bromo-2-(bromomethyl)-3-fluoropyridine (240 mg, 1.0 mmol) in dry DMF (2 mL) was dropwise added. The resulting mixture was stirred at 0° C. for 20 min and then rt overnight. The mixture was carefully quenched with water at 0° C., diluted and worked up with EtOAc-water. The combined organic layers were dried over MgSO₄, then residue DMF was removed under lyophilization. The crude was evaporated and subjected to ISCO purification using EtOAc-Hexanes (0-100%) to provide the product as a white solid (190 mg, 66%). LC-MS: m/z [M+H]⁺ 319.1, 321.1.

### Step 5: methyl 5-fluoro-6-((2-methyl-1H-benzo[d]imidazol-1-yl)methyl)nicotinate

The title compound was synthesized by following the same experimental procedure as described in Example 17 (step 2) employing 1-((5-bromo-3-fluoropyridin-2-yl)methyl)-2-methyl-1H-benzo[d]imidazole instead. LC-MS: m/z [M+H]⁺ 300.1.

### Step 6: 5-fluoro-N-hydroxy-6-((2-methyl-1H-benzo[d]imidazol-1-yl)methyl)nicotinamide

The title compound was synthesized by following the same experimental procedure as described in Example 17 employing methyl 5-fluoro-6-((2-methyl-1H-benzo[d]imidazol yl)methyl)nicotinate instead.

### Analysis of 5-fluoro-N-hydroxy-6-((2-methyl-1H-benzo[d]imidazol-1-yl)methyl)nicotinamide

¹H NMR (400 MHz, DMSO-d6) δ 11.35 (br, s, 1H), 9.25 (s, 1H), 8.59 (s, 1H), 8.01 (m, 1H), 7.51 (m, 1H), 7.39 (m, 1H), 7.13 (m, 2H), 5.68 (s, 2H).

LC-MS: m/z [M+H]⁺ C₁₅H₁₃FN₄O₂ requires: 300.1, found: 301.1

HPLC tR (min) 1.65, 98% (10-100% ACN with 0.1% TFA 10 min.)

### Example 31

### Preparation of 5-fluoro-N-hydroxy-6-((3-oxo-2,3-dihydro-4H-benzo[b][1,4]oxazin-4-yl)methyl)nicatinamide (IV-7)

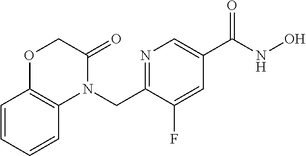 [SMILES: O=C(NO)c1cnc(CN2C(=O)COc3ccccc32)c(F)c1]

The title compound was synthesized by following the same experimental procedure as described in Example 30 employing 2H-benzo[b][1,4]oxazin-3(4H)-one instead.

### Analysis of 5-fluoro-N-hydroxy-6-((3-oxo-2,3-dihydro-4H-benzo[b][1,4]oxazin-4-yl)methyl)nicotinamide

¹H NMR (400 MHz, DMSO-d6) δ 11.39 (br, s, 1H), 9.28 (s, 1H), 8.59 (s, 1H), 7.98 (m, 1H), 7.04-6.96 (m, 4H), 5.34 (s, 2H), 4.72 (s, 2H).

LC-MS: m/z [M+H]⁺ C₁₅H₁₂FN₃O₄ requires: 317.1, found: 318.1

HPLC tR (min) 4.76, 99% (10-100% ACN with 0.1% TFA 10 min.)

### Example 32

### Preparation of 6-((3,4-dihydroisoquinolin-2(1H)-yl)methyl-5-fluoro-N-hydroxynicotinamide (IV-8)

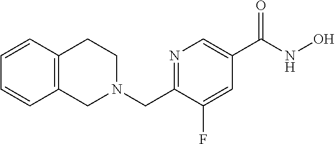 [SMILES: O=C(NO)c1cnc(CN2CCc3ccccc3C2)c(F)c1]

The title compound was synthesized by following the same experimental procedure as described in Example 30 employing 1,2,3,4-tetrahydroisoquinoline instead.

### Analysis of 6-((3,4-dihydroisoquinolin-2(1H)-yl)methyl)-5-fluoro-N-hydroxynicotinamide

¹H NMR (400 MHz, DMSO-d6) δ 11.45 (br, s, 1H), 9.41 (s, 1 H), 8.74 (s, 1H), 7.97 (m, 1H), 7.08-6.99 (m, 4H), 3.86 (s, 2H), 3.62 (s, 2H), 2.77 (m, 4H).

LC-MS: m/z [M+H]⁺ C16H16FN3O2 requires: 301.1, found: 302.1

HPLC tR, (min) 4.63, 97% (10-100% ACN with 0.1% TFA 10 min.)

### Example 33

### Preparation of 5-fluoro-N-hydroxy-6-((1-(pyridin-2-yl)cyclopropyl)amino)nicotinamide (I-18)

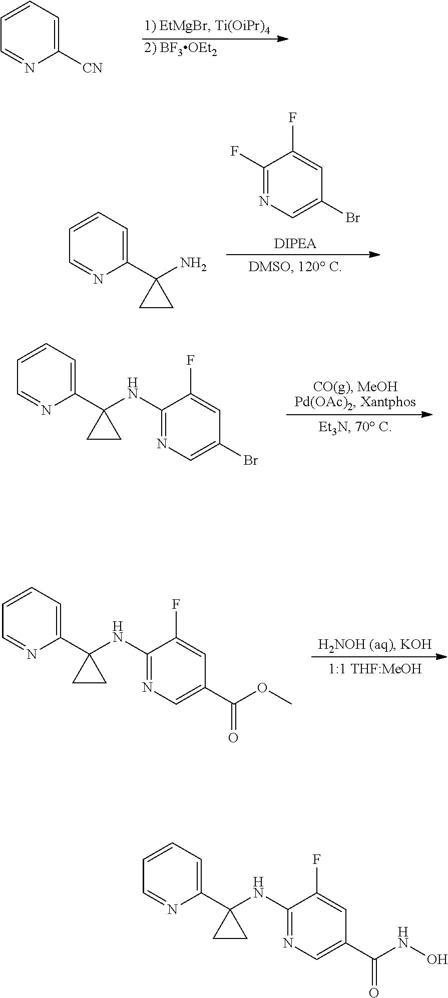

### Step 1: 1-(pyridin-2-yl)cyclopropan-1-amine

To an oven-dried 200 mL round-bottom flask containing a 1.5-inch egg-shaped stirbar under N₂ atmosphere was added pyridine-2-carbonitrile (2.08 g, 20 mmol), followed by THF (80 mL). To the pale yellow solution was added titanium tetraisopropoxide (7.1 mL, 24 mmol, 1.2 equiv) all at once, yielding no visible changes. Ethylmagnesium bromide (3M in ether, 16.7 mL, 50 mmol, 2.5 equiv) was added dropwise over the course of 10 minutes with vigorous stirring, immediately yielding a viscous black opaque suspension (caution: exothermic, gas evolution). After stirring for 90 minutes at room temperature, the black suspension was cooled to 0° C. in an ice bath. Then, BF₃.OEt₂ (4.9 mL, 40 mmol, 2 equiv) was added dropwise over the course of 5 minutes (caution: exothermic, gas evolution). The ice bath was removed and the viscous black suspension was allowed to warm to room temperature overnight. The following day, the reaction was quenched by adding 1M NaOH (100 mL, 5 equiv) in small portions at first, followed by ethyl acetate (50 mL), and then stirred vigorously at room temperature for 2 hours to yield a biphasic mixture of a top orange organic layer and a bottom black aqueous emulsion. This biphasic mixture was filtered directly through water-wetted celite, washed once with water (50 mL) and once with ethyl acetate (50 mL). The filtrate was collected and the layers separated. The aqueous layer was extracted twice more with ethyl acetate (50 mL). The combined organic layers were washed twice with water (50 mL) and once with brine (25 mL), then dried over MgSO₄, filtered and concentrated by rotary evaporation. The crude product was purified by column chromatography (Silica gel, 0-10% methanol in DCM with 1% NH₄OH) to afford the title compound as a brown oil, 346 mg (13%). LC-MS: m/z [M+H]⁺ 135.1.

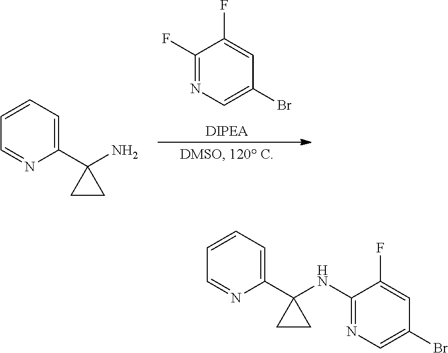

### Step 2: 5-bromo-3-fluoro-N-(1-(pyridin-2-yl)cyclopropyl)pyridin-2-amine

To a vial was added 1-(pyridin-2-yl)cyclopropan-1-amine (171 mg, 1.27 mmol), DMSO (3 mL), DIPEA (1.1 mL, 6.4 mmol, 5 equiv), and 5-bromo-2,3-difluoropyridine (0.26 mL, 1.9 mmol, 1.5 equiv). The biphasic homogeneous mixture was heated to 120° C. overnight under N₂ atmosphere (balloon). Upon reaching 120° C., the reaction becomes monophasic. The following day, LCMS analysis of the dark brown mixture reveals full conversion of the amine partner. The reaction was worked up by pouring into water (50 mL) and extracting three times with EtOAc (30 mL each). The combined organic layers were washed twice with water and once with brine, then dried over MgSO₄, filtered and concentrated by rotary evaporation. The crude product, a brown oil, was dry-loaded onto silica gel and purified by column chromatography (Silica gel, 0-50% EtOAc/hexanes) to afford the title compound as a yellow oil that solidifies into a beige solid at room temperature, 185.3 mg (47%). LC-MS: m/z [M+H]⁺ 308.1, 310.1 (1:1 ratio).

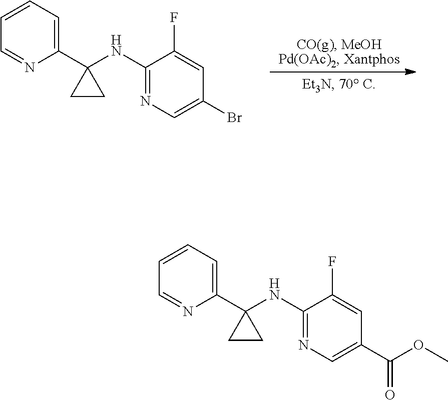 [SMILES: Fc1cc(Br)cnc1NC1(c2ccccn2)CC1 ; "CO(g), MeOHPd(OAc)2, Xantphos" ; "Et3N, 70° C." ; COC(=O)c1cnc(NC2(c3ccccn3)CC2)c(F)c1]

### Step 3: methyl 5-fluoro-6-((1-(pyridin-2-yl)cyclopropyl)amino)nicotinate

To a vial was added 5-bromo-3-fluoro-N-(1-(pyridin-2-yl)cyclopropyl)pyridin amine (85.0 mg, 0.28 mmol), followed by palladium diacetate (1.2 mg, 5 μmol, 0.02 equiv) and Xantphos (6.4 mg, 11 μmol, 0.04 equiv). Then, triethylamine (2 mL) and methanol (0.5 mL) were added, yielding a dark yellow heterogeneous suspension. The reaction mixture was sparged with a CO balloon for 1 minute, then the reaction mixture was heated to 70° C. overnight under a static CO atmosphere (CO balloon). The following day, LCMS analysis of the gray heterogeneous mixture reveals complete conversion of the bromide starting material. The reaction was worked up by dry-loading onto silica gel directly: the reaction was diluted with ethyl acetate (5 mL), silica gel (2 g) was added, and all volatiles were removed by rotary evaporation. The material was purified by column chromatography (Silica gel, 0-50% EtOAc/hexanes) to afford the title compound as a colorless oil, 51.7 mg (65%). LC-MS: m/z [M+H]⁺ 288.1.

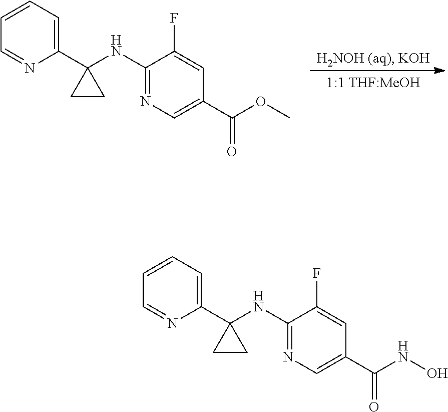 [SMILES: COC(=O)c1cnc(NC2(c3ccccn3)CC2)c(F)c1 ; "1:1 THF:MeOH" ; "H2NOH (aq), KOH" ; O=C(NO)c1cnc(NC2(c3ccccn3)CC2)c(F)c1]

### Step 4: 5-fluoro-N-hydroxy-6-((1-(pyridin-2-yl)cyclopropyl)amino)nicotinamide

To a vial was added methyl 5-fluoro-6-((1-(pyridin-2-yl)cyclopropyl)amino)nicotinate (28.3 mg, 0.098 mmol), followed by methanol (1 mL) and THF mL). The colorless homogeneous solution was cooled to 0° C. in an ice bath. Then, 50% aqueous hydroxylamine (0.18 mL, 3.0 mmol, 30 equiv) was added all at once. Lastly, one pellet of KOH (55 mg, 1.0 mmol, 10 equiv) was added. The vial was capped, and the ice bath was removed. The reaction was allowed to warm to room temperature with vigorous stirring. After 1 hour, the reaction was quenched by addition of 1M HCl (3 mL, 30 equiv). The pH was tested via colorimetric strip and found to be pH 5-6. The reaction was poured into 50 mL half-saturated aqueous —NaHCO₃, then extracted three times with EtOAc (30 mL). The combined organic layers were washed once with half-saturated NaHCO₃ (50 mL), once with brine (30 mL), then dried over MgSO₄, filtered and concentrated by rotary evaporation to afford the title compound as a pale yellow oil, 26.9 mg (95%) of high analytical purity.

### Analysis of 5-fluoro-N-hydroxy-6-((1-(pyridin yl)cyclopropyl)amino)nicotinamide

¹H NMR (400 MHz, d6-DMSO), δ ppm 10.98 (br s, 1H) 8.94 (br s, 1H) 8.43 (d, 1=4.16 Hz, 1H) 8.17 (s, 1H) 8.09 (s, 1H) 7.67 (d, J=11.98 Hz, 1H) 7.59 (t, J=7.59 Hz, 1H) 7.24 (d, J=7.83 Hz, 1 H) 7.11 (dd, J=6.48, 5.26 Hz, 1 H) 1.51-1.64 (m, 2H) 1.24 (br d, J=2.69 Hz, 2 H).

LC-MS: m/z [M+H]⁺ C₁₄H₁₃FN₄O₂ requires: 288.1, found: 289.1.

HPLC tR (min) 1.41, 97.8% (10-100% MeCN/H₂O with 0.1% TFA, 10 min)

### Example 34

### Preparation of 6-((1-(2,6-difluorophenyl)cyclopropyl)amino)-5-fluoro-N-hydroxynicotinamide (I-49)

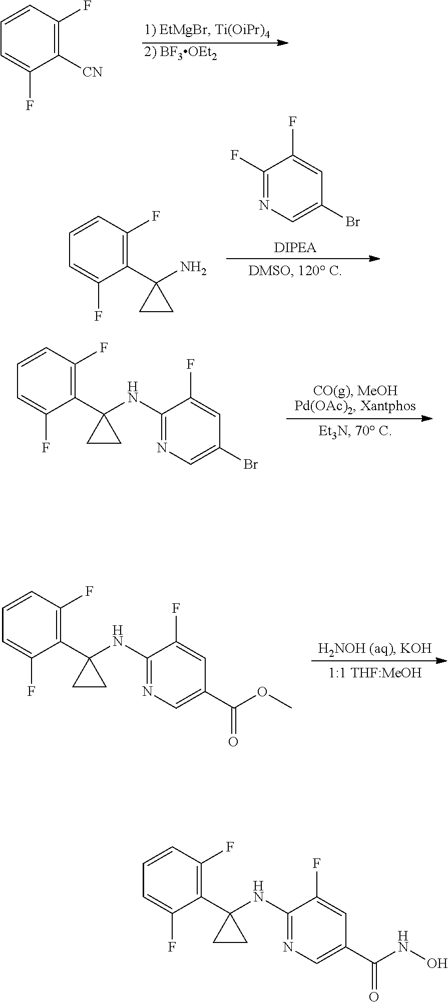

### Step 1: 1-(2,6-difluorophenyl)cyclopropan-1-amine

To an oven-dried 200 mL round-bottom flask containing a 1.5-inch egg-shaped stirbar under N₂ atmosphere was added 2,6-difluorohenzonitrile (2.78 g, 2 mmol), followed by methyl tert-butyl ether (MTBE) (100 mL). The pale yellow solution was cooled to −78° C. Titanium tetraisopropoxide (7.3 mL, 24 mmol, 1.2 equiv) was added all at once, yielding no visible changes. Ethylmagnesium bromide (3M in ether, 16.7 mL, 50 mmol, 2.5 equiv) was added dropwise over the course of 5 minutes with vigorous stilling, yielding a pale yellow homogeneous solution (caution: potential for exotherm and gas evolution). No gas evolution was observed in this case. The dry ice bath was left to expire slowly over the course of 4 hours with vigorous stirring (1500 RPM) of the solution. Upon warming to room temperature, a viscous opaque brown solution is formed. This solution was cooled to 0° C. in an ice bath. Then, BF₃.OEt₂ (4.9 mL, 40 mmol, 2 equiv) was added dropwise over the course of 5 minutes (caution: exothermic, gas evolution). The ice bath was removed and the opaque brown suspension was allowed to warm to room temperature overnight. The following day, the reaction was quenched by adding 1M NaOH (100 mL, 5 equiv) in small portions at first, followed by ethyl acetate (50 mL), and then stirred vigorously at room temperature for 2 hours to yield a biphasic mixture of a top colorless organic layer and a bottom dark blue aqueous emulsion. This biphasic mixture was filtered directly through water-wetted celite, washed once with water (50 mL) and once with ethyl acetate (50 mL). The filtrate was collected and the layers separated. The aqueous layer was extracted twice more with ethyl acetate (50 mL). The combined organic layers were washed twice with water (50 mL) and once with brine (25 mL), then dried over MgSO₄, filtered and concentrated by rotary evaporation. The crude product was purified by column chromatography (Silica gel, 0-50% ethyl acetate in hexanes) to afford the title compound as a pale yellow oil, 1.66 g (49%). LC-MS: m/z 170.1.

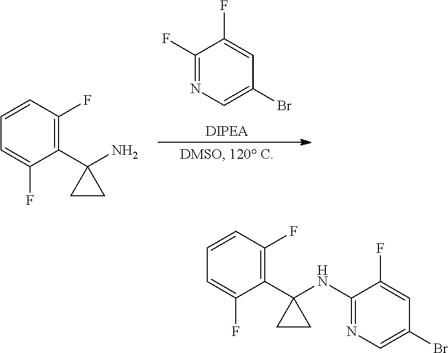

### Step 2: 5-bromo-N-(1-(2,6-difluorophenyl)cyclopropyl)-3-fluoropyridin-2-amine

To a vial was added 1-(2,6-difluorophenyl)cyclopropan-1-amine (304 mg, 1.80 mmol), DMSO (3 mL), DIPEA (1.57 mL, 9.0 mmol, 5 equiv), and 5-bromo-2,3-difluoropyridine (0.29 mL, 2.2 mmol, 1.2 equiv), The biphasic homogeneous mixture was heated to 120° C. overnight under N₂ atmosphere (balloon). Upon reaching 120° C., the reaction becomes monophasic. The following day, LCMS analysis of the dark brown mixture reveals full conversion of the amine partner. The reaction was worked up by pouring into water (50 mL) and extracting three times with EtOAc (30 mL each). The combined organic layers were washed twice with water and once with brine, then dried over MgSO₄, filtered and concentrated by rotary evaporation. The crude product, a brown oil, was dry-loaded onto silica gel and purified by column chromatography (Silica gel, 0-25% EtOAc/hexanes) to afford the title compound as a pale yellow oil, 400.5 mg (65%). LC-MS: does not ionize. ¹H NMR (CDCl₃): δ ppm 8.00 (s, 1H) 7.22. (dd, J=10.15, 1.83 Hz, 1H) 7.10-7.19 (m, 1H) 6.81 (t, J=8.07 Hz, 2H) 5.53 (br s, 1H) 1.31 (s, 4H).

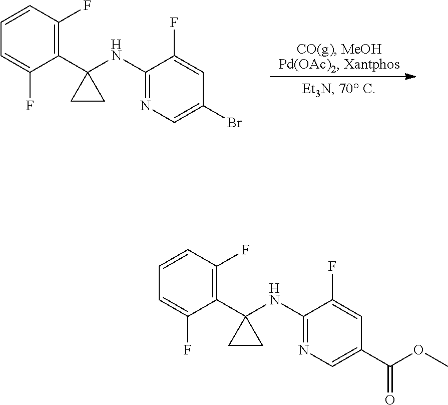 [SMILES: Fc1cc(Br)cnc1NC1(c2c(F)cccc2F)CC1 ; "CO(g), MeOHPd(OAc)2, Xantphos" ; "Et3N, 70° C." ; COC(=O)c1cnc(NC2(c3c(F)cccc3F)CC2)c(F)c1]

### Step 3: methyl 6-((1-(2,6-difluorophenyl)cyclopropyl)amino)-5-fluoronicotinate

To a vial was added 5-bromo-N-(1-(2,6-difluorophenyl)cyclopropyl)-3-fluoropyridin-2-amine (110 mg, 0.32 mmol), followed by palladium diacetate (1.4 mg, 6 μmol, 0.02 equiv) and Xantphos (7.4 mg, 13 μmol, 0.04 equiv). Then, triethylamine (2 mL) and methanol (0.5 mL) were added, yielding a pale yellow heterogeneous suspension. The reaction mixture was sparged with a CO balloon for 1 minute, then the reaction mixture was heated to 70° C. overnight under a static CO atmosphere (CO balloon). The following day, LCMS analysis of the gray heterogeneous mixture reveals complete conversion of the bromide starting material. The reaction was worked up by dry-loading onto silica gel directly: the reaction was diluted with ethyl acetate (5 mL), silica gel (2 g) was added, and all volatiles were removed by rotary evaporation. The material was purified by column chromatography (Silica gel, 0-40% EtOAc/hexanes) to afford the title compound as a colorless oil, 73.6 mg (72%). LC-MS: m/z [M+H]⁺ 323.1.

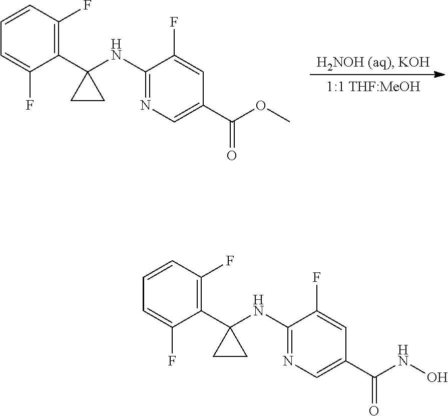 [SMILES: COC(=O)c1cnc(NC2(c3c(F)cccc3F)CC2)c(F)c1 ; "1:1 THF:MeOH" ; "H2NOH (aq), KOH" ; O=C(NO)c1cnc(NC2(c3c(F)cccc3F)CC2)c(F)c1]

### Step 4: 6-((1-(2,6-difluorophenyl)cyclopropyl)amino)-5-fluoro-N-hydroxynicotinamide

To a vial was added methyl 6-((1-(2,6-difluorophenyl)cyclopropyl)amino)-5-fluoronicotinate (30.5 mg, 0.095 mmol), followed by methanol (1 mL) and THF (1 mL). The colorless homogeneous solution was cooled to 0° C. in an ice bath. Then, 50% aqueous hydroxylamine (0.17 mL, 2.8 mmol, 30 equiv) was added all at once. Lastly, one pellet of KOH (53 mg, 0.95 mmol, 10 equiv) was added. The vial was capped, and the ice bath was removed. The reaction was allowed to warm to room temperature with vigorous stirring. After 1 hour, the reaction was quenched by addition of 1M HO (3 mL, 30 equiv). The pH was tested via colorimetric strip and found to be 5-6. The reaction was poured into 50 mL half-saturated aqueous NaHCO₃, then extracted three times with EtOAc (30 mL). The combined organic layers were washed once with half-saturated NaHCO₃ (50 mL), once with brine (30 mL), then dried over MgSO₄, filtered and concentrated by rotary evaporation to afford the title compound as a pale yellow oil, 37.6 mg (117%) of high analytical purity except for trace residual dichloromethane.

### Analysis of 6-((1-(2,6-difluorophenyl)cyclopropyl)amino)-5-fluoro-N-hydroxynicotinamide

¹H NMR (400 MHz, d6-DMSO) δ ppm 10.9 (br s, 1H) 8.91 (br s, 1H) 8.23 (s, 1H) 7.82 (s, 1H) 7.56 (d, J=12.23 Hz, 1 H) 7.28 (quin, J=7.40 Hz, 1H) 6.97 (t, J=8.31 Hz, 2H) 1.26-1.35 (m, 2H) 1.14-1.23 (m, 2H).

LC-MS: m/z [M+H]⁺ C₁₅H₁₂F₃N₃O₂ requires: 323.1, found: 324.1.

HPLC tR (min) 5.03, 95.5% (10-100% MeCN/H₂O with 0.1% TFA, 10 min)

### Example 35

### Preparation of 5-fluoro-N-hydroxy-6-((1-(pyridin-3-yl)cyclopropyl)amino)nicotinamide (I-20)

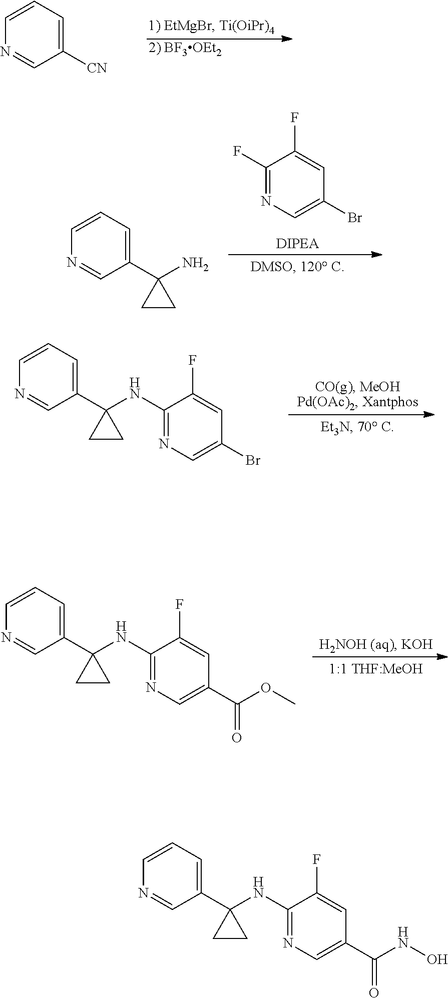

### Step 1: 1-(pyridin-3-yl)cyclopropan-1-amine

To an oven-dried 200 mL round-bottom flask containing a 1.5-inch egg-shaped stirbar under N₂ atmosphere was added pyridine-3-carbonitrile (2.08 g, 20 mmol), followed by methyl tert-butyl ether (MTBE) (100 mL). The colorless homogeneous solution was cooled to 78° C. Ethylmagnesium bromide (3M in ether, 16.7 mL, 50 mmol, 2.5 equiv) was added dropwise over the course of 5 minutes with vigorous stirring (caution: potential for exotherm and gas evolution). Titanium tetraisopropoxide (7.3 mL, 24 mmol, 1.2 equiv) was added dropwise over 5 minutes. No gas evolution was observed in this case. A bright orange solution was formed. The dry ice bath was removed after the addition of the above reagents. Upon warming to room temperature; a viscous opaque brown suspension is formed. After stirring at room temperature for 3 hours, the suspension was cooled to 0° C. in an ice bath. Then, BF₃.OEt₂ (4.9 mL, 40 mmol, 2 equiv) was added drop-wise over the course of 5 minutes (caution: exothermic, gas evolution). The ice bath was removed and the opaque brown suspension was allowed to warm to room temperature overnight. The following day, the reaction was quenched by adding 1M —NaOH (100 mL, 5 equiv) in small portions at first; followed by ethyl acetate (50 mL); and then stirred vigorously at room temperature for 2 hours to yield a biphasic mixture of a top pale yellow organic layer and a bottom yellow aqueous emulsion. This biphasic mixture was filtered directly through water-wetted celite, washed once with water (50 mL) and once with ethyl acetate (50 mL). The filtrate was collected and the layers separated. The aqueous layer was extracted twice more with ethyl acetate (50 mL), The combined organic layers were washed twice with water (50 mL) and once with brine (25 mL), then dried over MgSO₄, filtered and concentrated by rotary evaporation. The crude product was purified by column chromatography (Silica gel, 0-10% methanol in DCM with 1% MH₄OH) to afford the title compound as an orange oil, 822 mg (31%). LC-MS: m/z [M+H]⁺ 135.2.

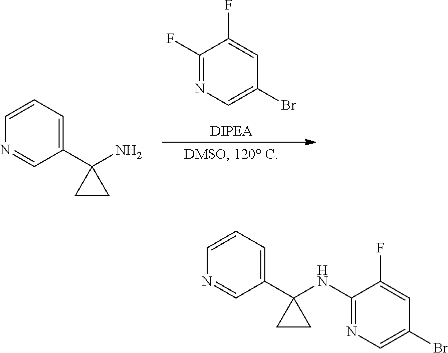

### Step 2: 5-bromo-3-fluoro-N-(1-(pyridin-3-yl)cyclopropyl)pyridin-2-amine

To a vial was added 1-(pyridin-3-yl)cyclopropan-1-amine (134 mg, 1.0 mmol), DMSO (3 mL), DIPEA (0.87 mL, 5 mmol, 5 equiv), and 5-bromo-2,3-difluoropyridine (0.2.0 mL, 1.5 mmol, 1.5 equiv). The biphasic homogeneous mixture was heated to 120° C. overnight under N2 atmosphere (balloon). Upon reaching 120° C., the reaction becomes monophasic. The following day, LCMS analysis of the dark brown mixture reveals full conversion of the amine partner. The reaction was worked up by pouring into water (50 mL) and extracting three times with EtOAc (30 mL each). The combined organic layers were washed twice with water and once with brine, then dried over MgSO₄, filtered and concentrated by rotary evaporation. The crude product, a brown oil, was dry-loaded onto silica gel and purified by column chromatography (Silica gel, 0-100% EtOAc/hexanes) to afford the title compound as a pale orange oil, 127 mg (41%). LC-MS: m/z [M+H]⁺ 308.1, 310.1 (1:1 ratio).

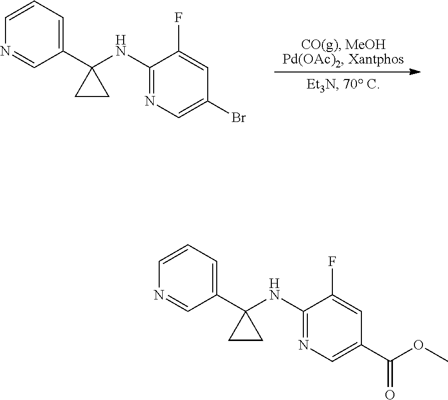 [SMILES: Fc1cc(Br)cnc1NC1(c2cccnc2)CC1 ; "CO(g), MeOHPd(OAc)2, Xantphos" ; "Et3N, 70° C." ; COC(=O)c1cnc(NC2(c3cccnc3)CC2)c(F)c1]

### Step 3: methyl 5-fluoro-6-((1-(pyridin-3-yl)cyclopropyl)amino)nicotinate

To a vial was added 5-bromo-3-fluoro-N-(1-(pyridin-3-yl)cyclopropyl)pyridin-2-amine (44.6 mg, 0.15 mmol), followed by palladium diacetate (0.6 mg, 3 μmol, 0.02 equiv) and Xantphos (3.3 mg, 6 μmol, 0.04 equiv). Then, triethylamine (2 mL) and methanol (0.5 mL) were added, yielding a pale yellow heterogeneous suspension. The reaction mixture was sparged with a CO balloon for 1 minute, then the reaction mixture was heated to 70° C. overnight under a static CO atmosphere (CO balloon). The following day, LCMS analysis of the gray heterogeneous mixture reveals complete conversion of the bromide starting material. The reaction was worked up by dry-loading onto silica gel directly: the reaction was diluted with ethyl acetate (5 mL), gel (2 g) was added, and all volatiles were removed by rotary evaporation. The material was purified by column chromatography (Silica gel, 0-100% EtOAc/hexanes) to afford the title compound as a pale yellow waxy solid, 35.8 mg (86%). LC-MS: m/z [M+H]⁺ 288.1.

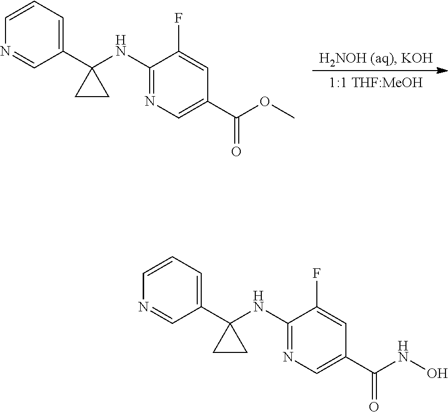 [SMILES: COC(=O)c1cnc(NC2(c3cccnc3)CC2)c(F)c1 ; "1:1 THF:MeOH" ; "H2NOH (aq), KOH" ; O=C(NO)c1cnc(NC2(c3cccnc3)CC2)c(F)c1]

### Step 4: 5-fluoro-N-hydroxy-6-((1-(pyridin-3-yl)cyclopropyl)amino)nicotinamide

To a vial was added methyl 5-fluoro-6-((1-(pyridin-3-yl)cyclopropyl)amino)nicatinate (35.8 mg, 0.13 mmol), followed by methanol (1 mL) and THF (1 mL). The colorless homogeneous solution was cooled to 0° C. in an ice bath. Then, 50% aqueous hydroxylamine (0.23 mL, 3.7 mmol, 30 equiv) was added all at once. Lastly, one pellet of KOH (70 mg, 1.3 mmol, 10 equiv) was added. The vial was capped, and the ice bath was removed. The reaction was allowed to warm to room temperature with vigorous stirring. After 1 hour, the reaction was quenched by addition of 1M HCl (3 mL, 30 equiv). The pH was tested via colorimetric strip and found to be pH 5-6. The reaction was poured into 50 mL half-saturated aqueous NaHCO₃, then extracted three times with EtOAc (30 mL). The combined organic layers were washed once with half-saturated NaHCO₃ (50 mL), once with brine (30 mL), then dried over MgSO₄, filtered and concentrated by rotary evaporation to afford the title compound as a white powder, 25.3 mg (70%) of high analytical purity.

¹H NMR (400 MHz, d₆-DMSO), δ ppm 11.01 (br s, 1 H) 8.99 (br s, 1H) 8.40 (br s, 1H) 8.33 (br d, J=3.42 Hz, 1H) 8.10-8.27 (two overlapping br s, 2H) 7.65 (br d, J=12.47 Hz, 1H) 7.54 (br d, J=7.34 Hz, 1 H) 7.23-7.30 (m, 1H) 1.36 s, 2H) 1.28 (br s, 2H).

LC-MS: m/z [M+H]⁺ C₁₄H₁₃FN₄O₂ requires: 288.1, found: 289.1.

HPLC tR (min) 1.49, 76.7% with protonation state shoulder artifact at 1.65, 20.4%. Sum of both peaks is 97.1%. (10-100% MeCN/H₂O with 0.1% TFA, 10 min)

### Example 36

### Preparation of 5-fluoro-N-hydroxy-6-((2-(pyridin-2-yl)propan-2-yl)amino)nicotinamide)

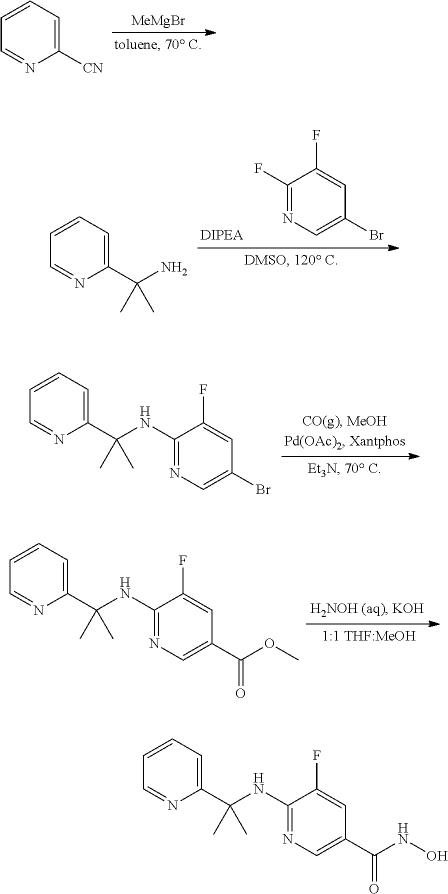

### Step 1: 2-(pyridin-2-yl)propan-2-amine

To a vial containing pyridine-2-carbonitrile (521 mg, 5.0 mmol) was added toluene (6 mL). The pale orange-brown homogeneous solution was cooled to 0° C. in an ice bath. Methylmagnesium bromide (1.4 M in 3:1 toluene: THF, 8.9 mL, 12.5 mmol, 2.5 equiv) was added drop-wise with vigorous stirring over 5 minutes. The resulting brown opaque solution was heated to 70° C. overnight under N₂ atmosphere (balloon). The following day, LCMS analysis indicated complete conversion. The black heterogeneous mixture was worked up by cooling to 0° C. in an ice bath, then adding 1M HCl (20 mL) (caution: exothermic, gas evolution). The lower brown aqueous layer was separated from the top clear toluene layer. The aqueous layer was basified (pH >10) using 1M NaOH (30 mL), then extracted three times with EtOAc (30 mL each). The combined organic layers were washed with water, then brine, then dried over MgSO₄ and filtered and concentrated by rotary evaporation to provide the title compound as a brown oil, 485 mg (71%), which was taken forward without further purification. LC-MS: m/z [M+H]⁺ 137.1.

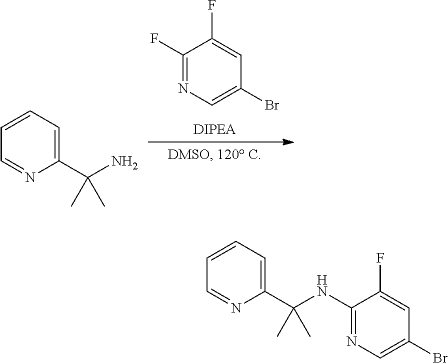

### Step 2: 5-bromo-3-fluoro-N-(2-(pyridin-2-yl)propan-2-yl)pyridin-2-amine

To a vial was added 2-(pyridin-2-yl)propan-2-amine (144 mg, 1.06 mmol), DMSO (2 mL), DIPEA (0.92 mL, 5.3 mmol, 5 equiv), and 5-bromo-2,3-difluoropyridine (0.21 mL, 1.6 mmol, 1.5 equiv). The biphasic homogeneous mixture was heated to 120° C. overnight under N₂ atmosphere (balloon). Upon reaching 120° C., the reaction becomes monophasic. The following day, LCMS analysis of the dark brown mixture reveals full conversion of the amine partner. The reaction was worked up by pouring into water (50 mL) and extracting three times with EtOAc (30 mL each). The combined organic layers were washed twice with water and once with brine, then dried over MgSO₄, filtered and concentrated by rotary evaporation. The crude product, a brown oil, was dry-loaded onto silica gel and purified by column chromatography (Silica gel, 0-25% EtOAc/hexanes) to afford the title compound as a yellow oil, 199.5 mg (61%). LC-MS: m/z [M+H]⁺ 310.1, 312.1 (1:1 ratio).

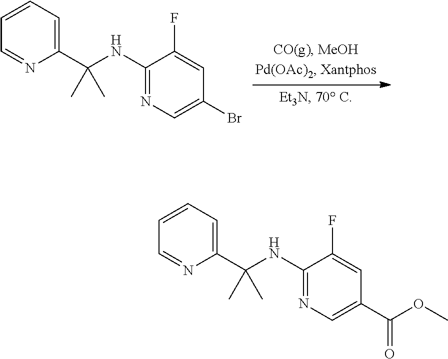 [SMILES: CC(C)(Nc1ncc(Br)cc1F)c1ccccn1 ; COC(=O)c1cnc(NC(C)(C)c2ccccn2)c(F)c1]

### Step 3: methyl 5-fluoro-6-((2-(pyridin-2-yl)propan-2-yl)amino)nicotinate

To a vial was added the oil 5-bromo-3-fluoro-N-(2-(pyridin-2-yl)propan-2-yl)pyridin-2-amine (63.4 mg, 0.20 mmol), followed by palladium diacetate (1 mg, 4 μmol, 0.02 equiv) and Xantphos (4.7 mg, 8 Mmol, 0.04 equiv). Then, triethylamine (2 mL) and methanol (0.5 mL) were added, yielding a pale yellow heterogeneous suspension. The reaction mixture was sparged with a CO balloon for 1 minute, then the reaction mixture was heated to 70° C. overnight under a static CO atmosphere (CO balloon). The following day, LCMS analysis of the gray heterogeneous mixture reveals complete conversion of the bromide starting material. The reaction was worked up by dry-loading onto silica gel directly: the reaction was diluted with ethyl acetate (5 mL), silica gel (2 g) was added, and all volatiles were removed by rotary evaporation. The material was purified by column chromatography (Silica gel, 0-30% EtOAc/hexanes) to afford the title compound as a colorless oil, 44.1 mg (75%). LC-MS: m/z [M+H]⁺ 290.1.

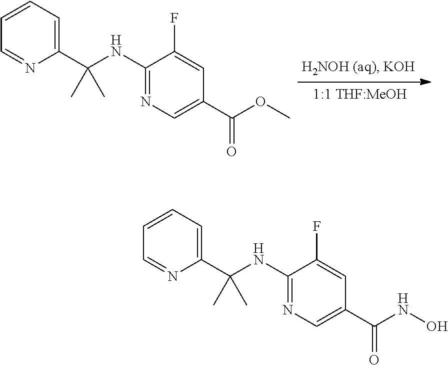 [SMILES: COC(=O)c1cnc(NC(C)(C)c2ccccn2)c(F)c1 ; CC(C)(Nc1ncc(C(=O)NO)cc1F)c1ccccn1]

### Step 4: 5-fluoro-N-hydroxy-6-((-2-(pyridin-2-yl)propan-2-yl)amino)nicotinamide

To a vial was added methyl 5-fluoro-6-((2-(pyridin-2-yl)propan-2-yl)amino)nicotinate (44.1 mg, 0.15 mmol), followed by methanol (1 mL) and THF (1 mL). The colorless homogeneous solution was cooled to 0° C. in an ice bath. Then, 50% aqueous hydroxylamine (0.28 mL, 4.5 mmol, 30 equiv) was added all at once. Lastly, one pellet of KOH (85 mg, 1.5 mmol, 10 equiv) was added. The vial was capped, and the ice bath was removed. The reaction was allowed to warm to room temperature with vigorous stirring. After 1 hour, the reaction was quenched by addition of 1M HCl (3 mL, 20 equiv). The pH was tested via colorimetric strip and found to be pH 5-6. The reaction was poured into 50 mL half-saturated aqueous NaHCO₃, then extracted three times with EtOAc (30 mL). The combined organic layers were washed once with half-saturated NaHCO₃ (50 mL), once with brine (30 mL), then dried over MgSO₄, filtered and concentrated by rotary evaporation to afford the title compound as a solid yellow powder, 37.7 mg (85%) of high analytical purity.

### Analysis of 5-fluoro-N-hydroxy-6-((2-(pyridin-2-yl)propan-2-yl)amino)nicotinamide

¹H NMR (400 MHz, d6-DMSO), δ ppm 10.92 (br s, 1H) 8.91 (s, 1H) 8.51 (d, J=4.65 Hz, 1 H) 8.06 (s, 1H) 7.71 (t, J=7.83 Hz, 1H) 7.63 (br d, J=12.47 Hz, 1H) 7.39-7.49 (m, 2H) 7.22 (dd, J=6.85, 5.38 Hz, 1H) 1.76 (s, 6H).

LC-MS: m/z C₁₄H₁₅FN₄O₂ requires: 290.1, found: 291.1.

HPLC tR (min) 3.67, 98.8% (10-100% MeCN/H₂O with 0.1% IT A, 10 min)

### Example 37

### Preparation of 5-fluoro-N-hydroxy-6-(methyl(1-phenylcyclopropyl) amino)nicotinamide (I-22)

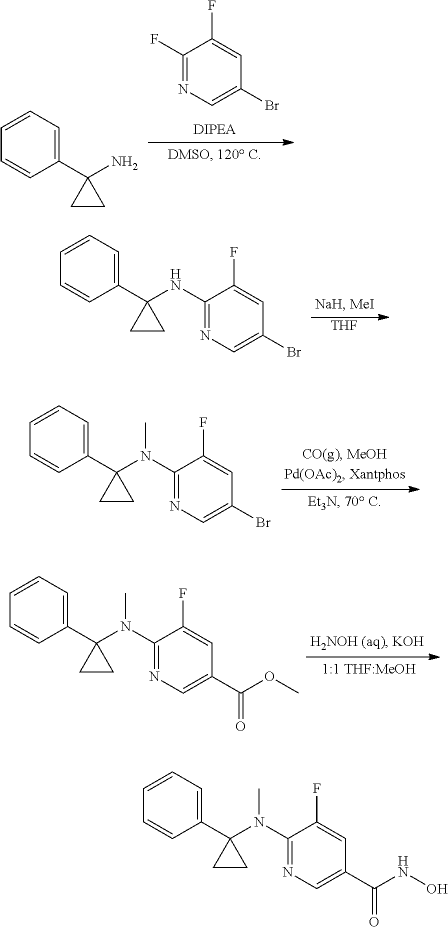

Step 1: 5-bromo-3-fluoro-N-(1-phenylcyclopropyl)pyridin-2-amine

To a vial was added 1-phenylcyclopropan-1-amine (244 mg, 1.83 mmol), DMSO (3 mL), DIPEA (1.7 mL, 10 mmol, 5 equiv), and 5-bronco-2,3-difluoropyridine (0.40 mL, 3.0 mmol, 1.5 equiv). The biphasic homogeneous mixture was heated to 120° C. overnight under N₂ atmosphere (balloon). Upon reaching 120° C., the reaction becomes monophasic. The following day, LCMS analysis of the dark orange mixture reveals full conversion of the amine partner. The reaction was worked up by pouring into water (50 mL) and extracting three times with EtOAc (30 mL each). The combined organic layers were washed twice with water and once with brine, then dried over MgSO₄, filtered and concentrated by rotary evaporation. The crude product, an orange oil, was dry-loaded onto silica gel and purified by column chromatography (Silica gel, 0- 20% EtOAc/hexanes) to afford the title compound as a grainy oil, which was triturated from hexanes (2 mL) at room temperature to yield a crystalline white solid, 208.1 mg (34%). LC-MS: m/z [M+H]⁺ 307.2, 309.2 (1:1 ratio).

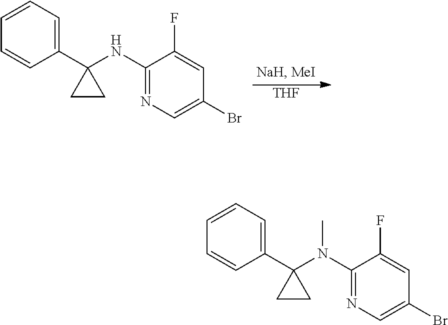 [SMILES: Fc1cc(Br)cnc1NC1(c2ccccc2)CC1 ; CN(c1ncc(Br)cc1F)C1(c2ccccc2)CC1 ; "THF"]

### Step 2: 5-bromo-3-fluoro-N-methyl-N-(1-phenylcyclopropyl)pyridin-2-amine

To a vial was added 5-bromo-3-fluoro-N-(1-phenylcyclopropyl)pyridin-2-amine (59.4 mg, 0.19 mmol) dissolved in THF (1 mL). The reaction was cooled to 0° C. in an ice bath, Sodium hydride (60 wt % in mineral oil, 40 mg, 1 mmol, 5 equiv) was added all at once, and the reaction was allowed to warm to room temperature for 10 minutes with vigorous stirring open to air (caution: gas evolution), yielding a beige suspension. Then, iodomethane (62 μL, 1 mmol, 5 equiv) was added all at once. The reaction was left to stir at room temperature for 2 hours, after which point TLC and LCMS analysis indicate complete, clean conversion. The reaction was worked up by pouring into water (50 mL), and then extracted three times with EtOAc (30 mL). The combined organic layers were washed with water (30 mL), then brine (30 mL), dried over MgSO₄, filtered and concentrated by rotary evaporation to yield the title product as a brown oil of reasonable purity, which was taken forward to the next step without further purification. LC-MS: m/z [M+H]⁺ 321.2, 323.2 (1:1 ratio).

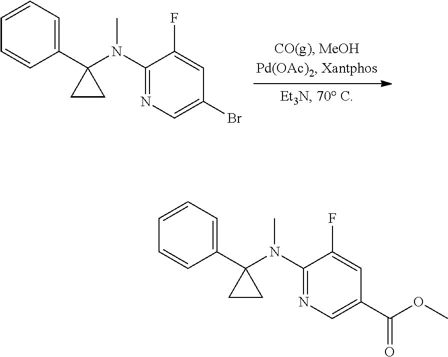 [SMILES: CN(c1ncc(Br)cc1F)C1(c2ccccc2)CC1 ; COC(=O)c1cnc(N(C)C2(c3ccccc3)CC2)c(F)c1]

### Step 3: methyl 5-fluoro-6-(methyl(1-phenylcyclopropyl)amino)nicotinate

To a vial was added 5-bromo-3-fluoro-N-methyl-N-(1-phenylcyclopropyl)pyridin-2-amine (69.5 mg, 0.22 mmol), followed by palladium diacetate (1 mg, 4 μmol, 0.02 equiv) and Xantphos (5.0 mg, 9 μmol, 0.04 equiv). Then, triethylamine (2 mL) and methanol (0.5 mL) were added, yielding an orange heterogeneous suspension. The reaction mixture was sparged with a CO balloon for 1 minute, then the reaction mixture was heated to 70° C. overnight under a static CO atmosphere (CO balloon). The following day, LCMS analysis of the gray heterogeneous mixture reveals complete conversion of the bromide starting material. The reaction was worked up by dry-loading onto silica gel directly: the reaction was diluted with ethyl acetate (5 mL), silica gel (2 g) was added, and all volatiles were removed by rotary evaporation. The material was purified by column chromatography (Silica gel, 0-15% EtOAc/hexanes) to afford the title compound as a colorless oil, 32.6 mg (50%). LC-MS: m/z [M+H]⁺ 301.1.

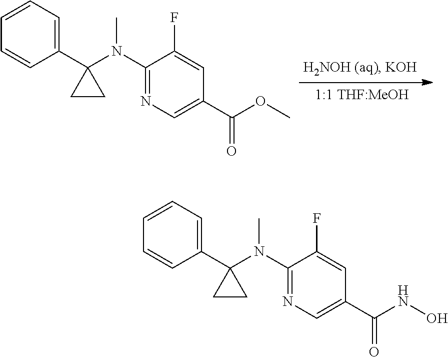 [SMILES: COC(=O)c1cnc(N(C)C2(c3ccccc3)CC2)c(F)c1 ; CN(c1ncc(C(=O)NO)cc1F)C1(c2ccccc2)CC1]

### Step 4: methyl 5-fluoro-6-(methyl(1-phenylcyclopropyl)amino)nicotinate

To a vial was added methyl 5-fluoro-6-((1-(pyridin yl)cyclopropyl)amino)nicotinate (32.6 mg, 0.11 mmol), followed by methanol (1 mL) and THF (1 mL). The colorless homogeneous solution was cooled to 0° C. in an ice bath. Then, 50% aqueous hydroxylamine (0.20 mL, 3.3 mmol, 30 equiv) was added all at once. Lastly, one pellet of KOH (61 mg, 1.1 mmol, 10 equiv) was added. The vial was capped, and the ice bath was removed. The reaction was allowed to warm to room temperature with vigorous stirring. After 1 hour, the reaction was quenched by addition of 1M HCl (3 mL, 30 equiv). The pH was tested via colorimetric strip and found to be pH 5-6. The reaction was poured into 50 mL half-saturated aqueous NaHCO₃, then extracted three times with EtOAc (30 nit). The combined organic layers were washed once with half-saturated NaHCO₃ (50 mL), once with brine (30 mL), then dried over MgSO₄, filtered and concentrated by rotary evaporation to afford the title compound as a pale yellow oil, 32.2 mg (98%) of high analytical purity.

### Analysis of methyl 5-fluoro-6-(methyl(1-phenylcyclopropyl)amino)nicotinate

¹H NMR (400 MHz, d6-DMSO), δ ppm 11.06 (br s, 1H) 9.00 (br s, 1H) 8.38 (s, 1H) 7.66 (br d, J=13.69 Hz, 1H) 7.27-7.34 (m, 2H) 7.15-7.21 (m, 1H) 7.08 (d, J=8.07 Hz, 2H) 3.18 (s, 3H) 1.38 (br d, J=6.85 Hz, 4H).

LC-MS: m/z [M+H]⁺ C₁₆H₁₆FN₃O₂ requires: 301.1, found: 302.1.

HPLC tR (min) 5.47, 99.0% (10-100% MeCN H₂O with 0.1% TFA, 10 min)

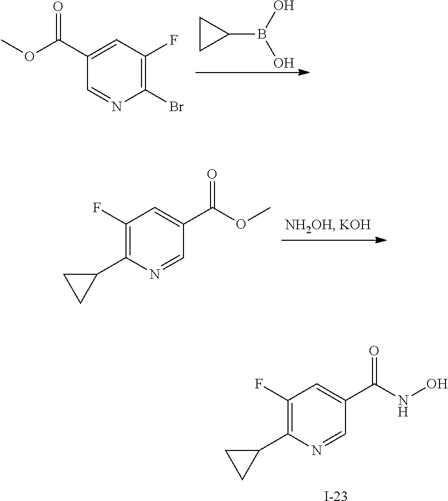

A mixture of methyl 6-bromo-5-fluoropyridine-3-carboxylate (70 mg, 0.3 mmol), cyclopropylboronic acid (128 mg, 1.5 mmol), potassium carbonate (62.0 mg, 0.45 mmol) in 1,4-dioxane (1.5 ML) was flushed with nitrogen for 10 min. [1,1′-Bis(diphenylphosphino)ferrocene]dichloropalladium(4) (43.8 mg, 0.06 mmol) was added and the mixture was flushed with nitrogen again for 5 min and heated in microwave at 110° C. for 4 h. LCMS showed complete conversion. The mixture was partitioned in ethyl acetate and water. The layers were separated. The aqueous layer was extracted with ethyl acetate (2×) and the combined organic layers were filtered over a pad of celite. The filtrate was concentrated to half of the volume and washed with water (3×), brine and concentrated to give 169 mg crude volatile oil. This material was purified by column chromatography (4 g SiO2, 0-10% ethyl acetate in hexane). Fractions containing the desired product were combined and collected to 55.3 mg (95%) colorless oil.

### Methyl 6-cyclopropyl-5-fluoropyridine-3-carboxylate

LCMS m/z [M+H]⁺ C₉H9FN₂O₂ requires: 195.07, found 195.1

1H NMR (400 MHz, CDCl₃) δ=8.85 (s, 1H), 7.85 (dd, J=9.90, 1.59 Hz, 1 H), 3.94 (s, 3H), 2.35-2.45 (m, 1H), 1.19-1.26 (m, 2H), 1.09-1.15 (m, 2H) ppm.

A mixture of methyl 6-cyclopropyl-5-fluoropyiidine-3-carboxylate (55.3 mg, 0.283 mmol), methanol (1 mL), and THF (1 mL) was cooled to 0° C. Hydroxylamine (0.52 mL, 50 wt % in water, 8.5 mmol) was added in a dropwise fashion, potassium hydroxide (80 mg, 1.42 mmol) was added in one portion, then reaction was stirred warming to room temperature. After 10 min, reaction complete by LCMS. Reaction mixture was concentrated under reduced pressure to remove organic solvents. The crude mixture was then diluted with water and neutralized to pH 7 with 1 M (aq). The product was extracted with EtOAc (3×), dried over Na₂SO₄, then concentrated to yield 39.1 mg (70%) product colorless solid.

### 6-cyclopropyl-5-fluoro-N-hydroxypyridine-3-carboxamide

LCMS m/z [M+H]⁺ C₉H₉FN₂O₂ requires: 196.06, found 197.1

HPLC method 2 Rt (min) purity 3.5, 99%

### 1H NMR (400 MHz, CD₃OD) δ=8.57 (s, 1H), 7.77 (d, J=10.27 Hz, 1H), 2.31-2.46 (m, 1H), 1.08-1.16 (m, 4H) ppm.

### Example 38

### Preparation of 5-fluoro-N-hydroxy-6-{[3-(pyridin-2-yl)oxetan-3-yl]oxy}pyridine-3-carboxamide (III-2)

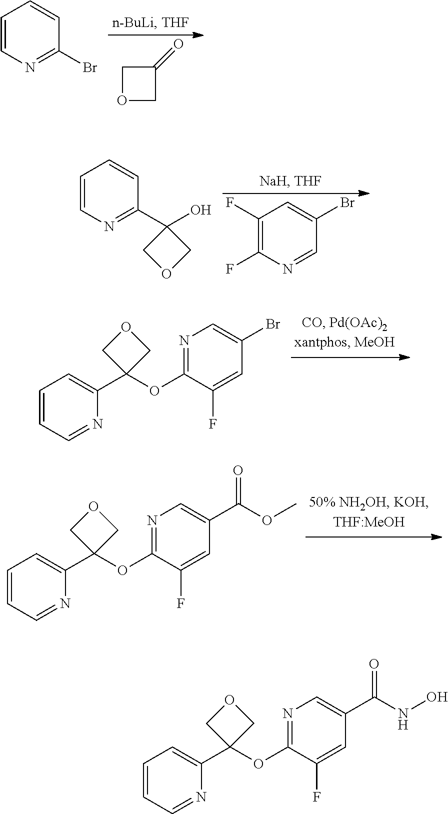

### Step 1: 3-(pyridin-2-yl)oxetan-3-ol

A solution of 2-bromopyridine (0.91 μL, 9.5 mmol) in 50 mL of THF was cooled to −78° C. and stirred for 30 min under nitrogen. 1 equiv of n-BuLi (2.5 M solution in hexanes, 4.5 mL, 11.4 mmol) was slowly added over a period of 5 min. The solution was stirred for 2 hrs at −78° C. after which oxetan-3-one (560 mL, 9.5 mmol) was added and the reaction solution was brought up to room temperature and stirred overnight under nitrogen. The reaction mixture was quenched with a saturated aqueous ammonium chloride solution and extracted with EtOAc. The organic layer was washed with brine and dried over MgSO₄. The organic layer was evaporated to an oil on a rotary evaporator that was dried onto silica. The product was purified by column chromatography using a Hex:EtOAc gradient 0-55%, to afford the title compound as a colorless oil, 517 mg (35%).

¹H NMR (400 MHz, CHLOROFORM-d) δ ppm 8.52 (br d, J=4.89 Hz, 1H) 8.00 (d, J=7.83 Hz, 1H) 7.89 (t, J=7.32 Hz, 1H) 7.33 (t. J=6.05 Hz. 1 H) 6.03 (s. 1 H) 5.11 (d, J=7.09 Hz, 2H) 4.74 (d, J=6.85 Hz, 2H)

### Step 2: 5-bromo-3-fluoro-2-{[3-(pyridin-2-yl)oxetan-3-yl]oxy}pyridine

3-(pyridin-2-yl)oxetan-3-ol (200 mg, 1.32 mmol) was dissolved in THF (13 mL) under nitrogen and cooled in an ice-bath. NaH (60? in mineral oil, 80 mg) was added in portions and the mixture stirred for 30-45 min. Then, 5-bromo-2,3-difluoropyridine (385 mg 1.98 mmol) was added dropwise and the mixture heated to 60° C. overnight. After completion, the reaction was cooled to room temperature and quenched with saturated ammonium chloride. The organic layer was separated and the aqueous layer was extracted with ethyl acetate (3×10 mL). The combined organic layers were dried over MgSO₄, filtered and concentrated. The product was purified by column chromatography using a Hex:EtOAc gradient 0-100% to afford the title compound as a colorless oil, 364 mg (85%).

1H NMR (400 MHz, CHLOROFORM-d) δ ppm 8.71 (d, J=5.09 Hz, 1H) 7.69-7.73 (m, 1H) 7.59-7.64 fin, 1H) 7.56 (dd, J=9.05, 1.96 HZ, 1H) 7.19-7.31 (m, 2H) 5.22 (d, J=7.58 Hz, 2H) 5.13 (d, J=7.34 Hz, 2H)

### Step 3: methyl 5-fluoro-6-{[3-(pyridin-2-yl)oxetan-3-yl]oxy}pyridine-3-carboxylate

A mixture of 5-bromo-3-fluoro-2-{[3-(pyridin-2-yl)oxetan-3-yl]oxy}pyridine (340 mg, 1.05 mmol), Palladium(II) acetate (9.47 mg, 0.0418 mmol) and Xantphos (48.4 mg, 0.0837 mmol) in MeOH (1.3 mL) and TFA (10 mL) was sparged with CO for 5-10 minutes and then heated to 72° C. overnight. After completion of reaction, the mixture was diluted with ethyl acetate and filtered through a pad of celite. The filtrate was washed with water followed by brine. The combined organic layers were dried over MgSO₄, filtered and concentrated. The product was purified by column chromatography using a Hex:EtOAc gradient 0-100% to afford the title compound as a white solid, 268 mg (85%).

¹H NMR (400 MHz, CHLOROFORM-d) δ ppm 8.71 (br d, J=4.65 Hz, 1H) 8.32 (s, 1H) 7.93-8.00 (m, 1H) 7.61 (t, J=7.83 HZ, 1H) 7.30 (d, J=8.07 HZ, 1H) 7.18-7.24 (m, 1H) 5.23-5.28 (m, 2H) 5.17 (d, J=7.34 Hz, 2H) 3.88 (s, 3H)

### Step 4: 5-fluoro-N-hydroxy-6-{[3-(pyridin-2-yl)oxetan-3-yl]oxy}pyridine-3-carboxamide

Methyl 5-fluoro-6-{[3-(pyridin-2-yl)oxetan-3-yl]oxy}pyridine-3-carboxylate (100 mg, 0.33 mmol) was dissolved in THF:MeOli (1:1, 3 mL) and cooled in an ice-bath with stirring. Hydroxylamine (50% solution in water, 0.3 mL, 9.89 mmol)) was added dropwise followed by KOH (94.2 mg, 1.65 mmol). The ice-bath was removed and the reaction stirred until complete consumption of starting material. After completion, the reaction mixture was concentrated, diluted with water and neutralized (pH=7) with 2N HCl. The aqueous layer was extracted with EtOAc (3×10 mL). The combined organic layers were dried over MgSO4, filtered and concentrated to afford the title compound as a white solid, 99 mg (99%).

### ¹H NMR (400 MHz, METHANOL-d4) δ ppm 8.60 (br d, J=4.16 Hz, 1H) 8.02 (s, 1H) 7.90 (br d, J=11.00 Hz, 1H) 7.71-7.81 (m, 1H) 7.45 (d, J=7.83 Hz, 1H) 7.27-7.35 (m, 1H) 5.18-5.23 (m, 2 H) 5.13 (d, J=7.34 Hz, 2H)

LC-MS: tR, (min) 4.20 (20-100% ACN with 0.1% TFA 6 min), m/z [M+H]⁺ C14H12FN3O4 requires: 305.27; found 306.0 HPLC tR (min) 4.04, 100% (20-100% ACN with 0.1% TFA 10 min.)

### Example 39

### Synthesis of 5-fluoro-N-hydroxy-6-4-(pyrimidin-2-yl)cyclopropyl)amino)nicotinamide (I-46)

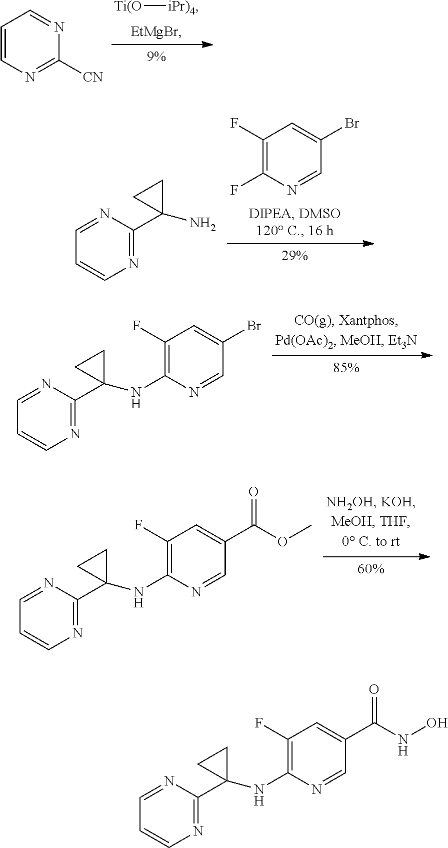

### 1-(pyrimidin-2-yl)cyclopropan-1-amine

To a cooled (15° C.) light suspension of pyrimidine-2-carbonitrile (5.00 g, 47.6 mmol) in dry THF (120 mL, 0.2 M) was added Titanium isopropoxide (14.9 g, 42.3 mmol, 15.9 mL), followed by ethyl magnesium bromide (20.8 g, 97.5 mmol, 32.5 mL) dropwise within 15 min. Towards the end, the reaction was slightly exothermic, addition rate was decreased accordingly. (inner temp ca. 50° C.). After complete addition the mixture was stirred for 45 min until TLC indicated complete conversion of the starting material. The mixture was quenched with water (20 mL), stirred for 10 min, then added with 2N NaOH (75 mL) and stirred for 20 min. The fine suspension was filtered through a pad of celite (slow filtration). The pad of celite was washed with ethyl acetate. More water was added to the filtrate. The layers were separated. The aqueous layer was extracted with ethyl acetate (2×). The combined organics were washed with brine, dried (Na₂SO₄) and concentrated to give 615 mg (10%) of 1-(pyrimidin-2-yl)cyclopropan-1-amine. ¹H NMR (400 MHz, CDCl₃) δ=8.62 (d, J=4.65 Hz, 2H), 7.05 (t, J=4.89 Hz, 1H), 2.05 (s, 2H), 1.42-1.48 (m, 2H), 1.20-1.24 (m, 2H) ppm

5-bromo-3-fluoro-N-(1-(pyrimidin-2-yl)cyclopropyl)pyridin-2-amine A mixture of 1-(pyrimidin-2-yl)cyclopropan-1-amine (202 mg, 1.49 mmol), 5-bromo-2,3-difluoropyridine (522 mg, 2.69 mmol) and DIPEA (7.47 mmol, 966 mg, 1.3 mL) in DMSO (6 mL) was stirred at 110° C. for 40 hours. After completion of the reaction as indicated by TLC the mixture was poured into water (60 mL) and extracted with ethyl acetate (3×). The combined organics were washed with water (3×), brine and dried (Na₂SO₄) and concentrated to give 551 mg brown oil. The crude material was purified by column chromatography (24 g SiO2, 0-30% EA in hexane). Fractions containing the desired product were combined and concentrated to give 132 mg (29%) 5-bromo-3-fluoro-N-(1-(pyrimidin-2-yl)cyclopropyl)pyridin-2-amine as yellow solid.

¹H NMR (400 MHz, CDCl₃) δ=8.56 (d, J=4.89 Hz, 2H), 7.88 (s, 1H), 7.28-7.37 (m, 1H), 7.02 (t, J=4.89 Hz, 1H), 5.63 (br s, 1H), 1.77-1.86 (m, 2H), 1.38-1.45 (in, 2H) ppm.

### Methyl 5-fluoro-6-((1-(pyrimidin-2-yl)cyclopropyl)amino)nicotinate

A mixture of 5-bromo-3-fluoro-N-[1-(pyrimidin-2-yl)cyclopropyl]pyridin-2-amine (112 mg, 0.362 mmol), Palladium(II) acetate (3.25 mg, 0.0145 mmol), Xantphos (16.8 mg, 0.029 mmol) in anhydrous triethylamine (3.62 mL, 0.1 M based on starting material) and anhydrous methanol (10.9 mmol, 1.07 mL, 30 equiv.) was flushed with CO (balloon) for 5 min with the needle being in the solution. After 5 min needle was taken out of the solution such that it hovered above the mixture and stirred at 70° C., After 16 hours LCMS indicated complete conversion. The mixture was partitioned in water and ethyl acetate. The layers were separated and the aqueous was extracted with ethyl acetate (2×). The combined organics were washed with water (2×), brine, dried (Na₂SO₄) and concentrated to give 113 mg crude material. It was purified by column chromatography (4 g SiO2, 0-60% EA in hexane). Fractions containing the desired product were combined and concentrated to give 89.1 mg (85%) methyl 5-fluoro-6-((1-(pyrimidin-2-yl)cyclopropyl)amino)nicotinate as off-white solid.

¹H NMR (400 MHz, CDCl₃) δ=8.55 (d, J=4.89 Hz, 2H), 8.52 (s, 1H), 7.68-7.76 (m, 1H), 7.03 (t, J=4.89 Hz, 1H), 5.99 (br s, 1H), 3.85 (s, 3H), 1.84-1.88 (m, 2H), 1.42-1.51 (m, 2H) ppm.

5-fluoro-N-hydroxy-6-((1-(pyrimidin-2-yl)cyclopropyl)amino)nicotinamide To a cooled solution (0° C.) of methyl 5-fluoro-6-{[1-(pyrimidin-2-yl)cyclopropyl]amino}pyridine-3-carboxylate (86 mg, 0.298 mmol) in methanol (2 mL) and THF (2 mL) was added dropwise NH₂OH (591 mg, 50% w/w, 8.95 mmol, 547 uL) followed by KOH in one portion (124 trig, 2.21 mmol), 5 min after complete addition the bath was removed, stirred at rt. After 15 min LCMS indicated complete conversion. The reaction mixture was concentrated under reduced pressure to remove organic solvents. The crude mixture was then diluted with water and neutralized to pH 7 by addition of 1 M HCl (aq). The product was extracted with EtOAc (3×), dried over Na₂SO₄ and concentrated to yield 52.1 mg (60%) 5-fluoro-N-hydroxy-6-((1-(pyrimidin-2-yl)cyclopropyl)amino)nicotinamide as pinkish solid.

¹H NMR (400 MHz, MeOD) δ=8.60 (d, J=4.89 Hz, 2H), 8.14 (s, 1 H), 7.58-7.69 (m, 1H), 7.21 (t, J=4.89 Hz, 1H), 1.78-1.86 (m, 2H), 1.40-1.47 (m, 2 H) ppm. LC-MS: tR (min) 1.10, m/z [M+H]+ C₁₃H₁₂FN₅O₂ require: 290.3, found 291.1 (20-100% ACN with 0.1% TFA 6 min.)

HPLC tR (min) 1.88, 99% (20-100% ACN with 0.1% TFA 10 min.)

### Example 40

### Preparation of N-cyclopropyl-1-((3-fluoro-5-(hydroxycarbamoyl)pyridin-2-yl)methyl)-1H-pyrrolo[2,3-b]pyridine-5-carboxamide (I-55)

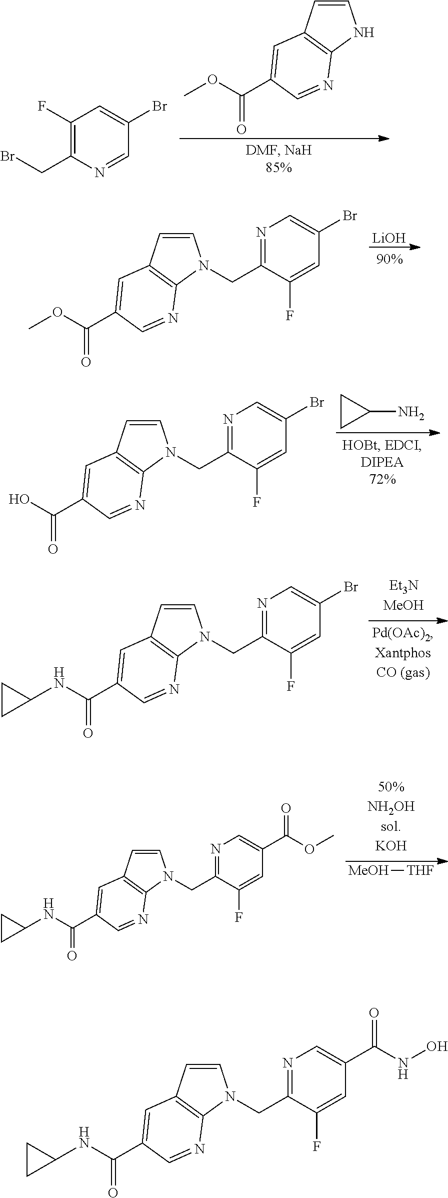

### Step 1: methyl 1-((5-bromo-3-fluoropyridin-2-yl)methyl)-1H-pyrrolo[2,3-b]pyridine-5-carboxylate

To a solution of methyl 1H-pyrrolo[2,3-b]pyridine-5-carboxylate (170 mg, 0.97 mmol) in dry DMF (3 mL) was added 60% NaH (38 mg, 0.97 mmol) at 0° C., then the mixture was stirred at 0° C. for 20 min. A solution of 5-bromo-2-(bromomethyl)-3-fluoropyridine (200 mg, 0.74 mmol) in dry DMF (2 mL) was dropwise added. The resulting mixture was stirred at 0° C. for 20 min and then rt overnight. The mixture was carefully quenched with water at 0° C., diluted and worked up with EtOAc-water. The combined organic layers were dried over MgSO₄, then residual DMF was removed under lyophilization. The crude was then subjected to ISCO purification using MeOH-DCM (0-10%) to provide the product as a white solid (230 mg, 85%). LC-MS: m/z [M+H]⁺ 364.0, 366.0.

### Step 2: 1-((5-bromo-3-fluoropyridin-2-yl)methyl)-1H-pyrrolo[2,3-b]pyridine-5-carboxylic acid

To a 20 mL of vial with methyl 1-((5-bromo-3-fluoropyridin-2-yl)methyl)-1H-pyrrolo[2,3-b]pyridine-5-carboxylate (230 mg, 0.63 mmol) were added LiOH.H₂O (53 mg, 2.1 mmol) and THF-MeOH—H₂O (v/v/v 1:1:1, 6 mL). The mixture was then stirred at 50° C. overnight. LC-MS showed completed conversion. The volatiles were evaporated. Then water (˜10 mL) was added, and 1 N HCl(˜2 mL) was added to adjust pH to 6. The white solid was filtered and washed with water (˜20 mL) and EtOAc (˜10 mL). (200 mg product obtained as a colorless solid after dried over lyophilization overnight). LC-MS: 350.1, 352.1.

### Step 3: 1-(((5-bromo-3-fluoropyridin-2-yl)methyl-N-cyclopropyl-1H-pyrrolo[2,3-b]pyridine-5-carboxamide

The crude acid from previous step (110 mg, 0.31 mmol) was dissolved in DMF (4 mL), then cyclopropanamine (21 mg, 0.37 mmol), EDCI (72 mg, 0.37 mmol), HOBt (51 mg, 0.37 mmol) and DIPEA (102 mg, 0.78 mmol) were added. The mixture was then allowed to stir at rt for 3 h, then poured into water (15 mL) and the product extracted with ethyl acetate (2×50 mL). The combined organic layers were dried over anhydrous sodium sulfate and concentrated in vacuo to provide the crude, which was purified by ISCO using 10% MeOH in DCM: DCM (0-60%) as eluent to afford the product as a white solid. LC-MS: 389.1, 391.1. ¹H NMR (400 MHz, CDCl₃) δ 8.04 (s, 1H), 7.38 (m, 1H), 6.59 (m, 1H), 4.99 (br s, 1H), 2.27 (m, 1H), 1.70 (m, 2H), 0.96 (m, 2H), 0.74 (m, 2H), 0.45 (in, 2H).

The last two steps followed the same experimental procedure as described in 5-fluoro-N-hydroxy-6-((2-methyl-1H-benzo[d]imidaziol-1-yl)methyl)nicotinamide employing 1-((5-bromo-3-fluoropyridin-2-yl)methyl)-N-cyclopropyl-1H-pyrrolo[2,3-b]pyridine-5-carboxamide instead.

### Analysis of N-cyclopropyl-1-((3-fluoro-5-(hydroxycarbamoyl)pyridin-2-yl)methyl)-1H-pyrrolo[2,3-b]pyri di ne-5-carboxamide

¹H NMR (400 MHz, DMSO-d6) δ 11.41 (br s, 1H), 9.33 (s, 1H), 8.64 (s, 1H), 8.56 (m, 1H), 8.46 (m, 1H), 8.41 (m, 1H), 8.00 (m, 1H), 7.67 (m, 1H), 6.63 (m, 1H), 5.75 (s, 2 H), 0.71 (m, 2 H), 0.58 (m, 2 H).

LC-MS: m/z [M+H]⁺ C18H16FN5O3 requires: 369.3, found: 370.1

HPLC tR (min) 4.73, 100% (10-100% ACN with 0.1% TFA 10 min.)

### Example 41

### Preparation of 5-fluoro-6-{[1-(3-fluoropyridin-2-yl)cyclopropyl]amino}-N-hydroxypyridine-3-carboxamide (I-45)

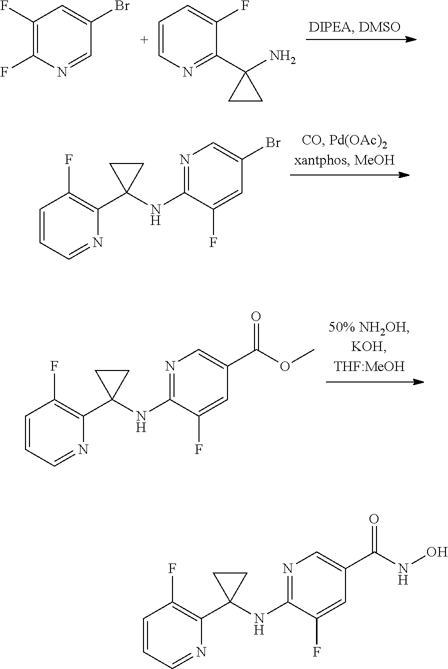

### Step 1: 5-bromo-3-fluoro-N-[1-(3-fluoropyridin-2-yl)cyclopropyl]pyridin-2-amine

N-Ethyldiisopropylamine (0.42 mL, 2.46) was added to a mixture of 5-bromo-2,3-difluoropyridine (191 mg, 0.986 mmol) and 1-(3-fluoropyridin-2-yl)cyclopropan-1-amine (75 mg, 0.493 mmol) in anhydrous DMSO (2.5 mL) under nitrogen. The reaction mixture was stirred at 110° C. overnight. After completion of reaction, the mixture was cooled, diluted with water and extracted with ethyl acetate (3×10 mL). The combined organic layers were dried over MgSO₄, filtered and concentrated. The product was purified by column chromatography using a Hex:EtOAc gradient 0-100% to afford the title compound as a yellow solid, 71 mg (44%).

¹H NMR (400 MHz, CHLOROFORM-d) δ ppm 8.30 (br d, J=4.65 Hz, 1 H) 7.92 (s, 1H) 7.21-7.32 (m, 3H) 7.12 (dt, J=8.31, 4.16 Hz, 1H) 5.66 (br s, 1H) 1.68-1.74 (m, 2H) 1.27-1.33 (m, 2H)

### Step 2: methyl 5-fluoro-6-{[1-(3-fluoropyridin-2-yl)cyclopropyl]amino}pyridine-3-carboxylate

A mixture of 5-bromo-3-fluoro-N-[1-(3-fluropropyridin-2-yl)cyclopropyl]pyridin-2-amine (71 mg, 0.218 mmol), Palladium(II) acetate (1.97 mg, 0.0087 mmol) and Xantphos (10.1 mg, 0.0174 mmol) in MeOH (0.5 mL) and TEA (2 mL) was sparged with CO for 5-10 minutes and then heated to 72° C. overnight. After completion of reaction, the mixture was diluted with ethyl acetate and filtered through a pad of celite. The filtrate was washed with water followed by brine. The combined organic layers were dried over MgSO₄, filtered and concentrated. The product was purified by column chromatography using a Hex:EtOAc gradient 0-100% to afford the title compound as a white solid, 49.2 mg (74%).

¹H NMR (400 MHz, CHLOROFORM-d) δ ppm 8.55 (s, 1H) 8.28-8.33 (m, 1H) 7.68 (dd, J=11.49, 0.73 Hz, 1H) 7.28 (br d, J=2.45 Hz, 1H) 7.12 (dt, J=8.25, 4.07 Hz, 1H) 6.06 (br s, 1H) 3.84 (s, 3H) 1.71-1.82 (m, 2 H) 1.24-1.40 (m, 3H)

### Step 3: 5-fluoro-6-{[1-(3-fluropropyridin-2-yl)cyclopropyl]amino}-N-hydroxypyridine-3-carboxamide

methyl 5-fluoro-6-{[1-(3-fluoropyridin-2-yl)cyclopropyl]amino}pyridine-3-carboxylate (48 mg, 0.157 mmol) was dissolved in THF:MeOH (1:1, 1 mL) and cooled in an ice-bath with stirring. Hydroxylamine (50% solution in water, 0.15 mL, 4.72 mmol)) was added dropwise followed by KOH (45 mg, 0.786 mmol). The ice-bath was removed and the reaction stirred until complete consumption of starting material. After completion, the reaction mixture was concentrated, diluted with water and neutralized (pH=7) with 2N HCl. The aqueous layer was extracted with EtOAc (3×10 mL), The combined organic layers were dried over MgSO₄, filtered and concentrated to afford the title compound as a white solid, 48 mg (99%).

¹H NMR (400 MHz, METHANOL-d4) δ ppm 8.21-8.27 (m, 1H) 8.14 (s, 1H) 7.58 (br d, J=12.96 Hz) 7.34-7.44 (m, 1 H) 7.17-7.24 (m, 1H) 1.69-1.75 (m, 2H) 1.27-1.32 (m, 3H)

LC-MS: tR (min) 4.20 (20-100% ACN with 0.1% TFA 6 min), m/z [M+H]⁺ C14H12F2N4O2 requires: 306.27; found 307.0

HPLC tR (min) 3.29, 98.3% (20-100% ACN with 0.1% TFA 10 min.)

In the following example compound I-37 was synthesized by following the same experimental procedure as described in Example 12 with the listed aldehyde starting material for step 1, and the listed organometallic reagent precursors for step 2.

| | Aldehyde/ | | |
|---|---|---|---|
| Ex. | Organometallic | Compound | Characterization Data |
| 42 | cyclobutanone/ 2-bromopyridine, and n-BuLi | 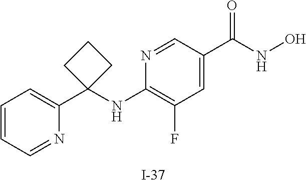 | ¹H NMR (400 MHz, MeOD-d4) δ 8.52 (d, J = 6.03 Hz, 1 H), 8.03 (s, 1 H), 7.70 (t, J = 7.70 Hz, 1 H), 7.58 (d, J = 12.2 Hz, 1 H), 7.51 (d, J = 8.07 Hz, 1 H), 7.22 (t, J = 6.03 Hz, 1 H), 2.89-2.80 (m, 2 H), 2.62- 2.52 (m, 2 H), 2.20-2.15 (m, 1 H), 2.12-2.06 (m, 1H). LC-MS t_{R} (min) 1.24 (20-100% ACN with 0.1% TFA 6 min), m/z [M + H]⁺ C₁₅H₁₅FN₄O₂ requires: 302.3, found: 303.1 HPLC t_{R} (min) 3.68, 95% (20-100% ACN with 0.1% TFA 10 min. |

### Examples 43-61

In the following examples the compounds were synthesized by following the same experimental procedure as described in Example 7 with the listed amine and methyl 5-fluoro-6-bromonicotinate starting material for step 1

| Ex. | Amine | Compound | Characterization Data |
|---|---|---|---|
| 43 | 2-amino- N, 2- dimethyl propanamide | 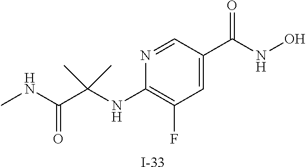 | ¹H NMR (400 MHz, MeOH-d4) δ 8.24 (s, 1 H), 7.62 (s, 1 H), 7.59 (s, 1 H), 2.69-2.65 (m, 3 H), 1.59 (s, 6 H). LC-MS: t_{R} (min) 1.32 (20-100% ACN with 0.1% TFA 6 min), m/z [M + H]⁺ C₁₁H₁₅FN₄O₂ requires: 270.3, found: 271.1 HPLC t_{R} (min) 1.57, 95% (20-100% ACN with 0.1% TFA 10 min.) |
| 44 | 3- amino-1- methyl- piperidin- 2-one | 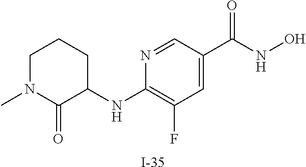 | ¹H NMR (400 MHz, DMSO-d6) δ 11.1-10.9 (br s, 1 H), 8.98 (s, 1 H), 8.25 (s, 1 H), 7.73-7.53 (m, 1 H), 7.29-7.04 (m, 1 H), 4.80-4.50 (m, 1 H), 3.33-3.28 (m, 2 H), 2.83 (s, 3 H), 2.16-2.00 (m, 1 H), 1.90 (s, 3 H). LC-MS: tR (min) 1.28 (20-100% ACN with 0.1% TFA 6 min), m/z [M + H]⁺ C₁₂H₁₅FN₄O₂ requires: 282.3, found: 283.1 HPLC tR (min) 3.66, 95% (20-100% ACN with 0.1% TFA 10 min.) |
| 45 | (S)-1- cyclo- propylethan- 1-amine | 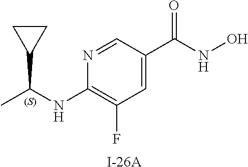 | ¹H NMR (400 MHz, MeOH-d4) δ 7.98 (s, 1 H), 7.32 (d, J = 1.71 Hz, 1 H), 7.29 (d, J = 1.71 Hz, 1 H), 3.45-3.35 (m, 1 H), 1.07 (d, J = 6.06 Hz, 3 H), 0.86-0.74 (m, 1 H), 0.33- 0.01 (m, 4 H). LC-MS: t_{R} (min) 2.03 (20-100% ACN with 0.1% TFA 6 min), m/z [M + H]⁺ C₁₁H₁₂FN₃O₂ requires: 239.3, found: 240.1 HPLC t_{R} (min) 4.37, 95% (20-100% ACN with 0.1% TFA 10 min.) |
| 46 | (R)-1- cyclo- propylethan- 1-amine | 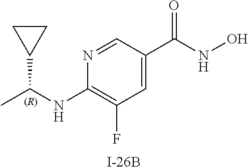 | ¹H NMR (400 MHz, MeOH-d4) δ 7.98 (s, 1 H), 7.32 (d, J = 1.71 Hz, 1 H), 7.29 (d, J = 1.47 Hz, 1 H), 3.10-3.07 (m, 1 H), 1.07 (d, J = 6.60 Hz, 3 H), 0.90-0.70 (m, 1 H), 0.33- 0.00 (m, 4 H). LC-MS: t_{R} (min) 2.03 (20-100% ACN with 0.1% TFA 6 min), m/z [M + H]⁺ C₁₁H₁₂FN₃O₂ requires: 239.3, found: 240.1 HPLC t_{R} (min) 3.74, 95% (20-100% ACN with 0.1% TFA 10 min.) |
| 47 | (R)-1- phenyl- propan-1- amine | 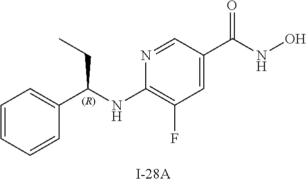 | ¹H NMR (400 MHz, MeOH-d4) δ 8.20 (s, 1 H), 7.56 (d, J = 11.7 Hz, 1 H), 7.39 (d, J = 7.58 Hz, 2 H), 7.29 (t, J = 7.46 Hz, 2 H), 7.24-7.11 (m, 1 H), 5.15-5.02 (m, 1 H), 2.00- 1.75 (m, 2 H), 1.02-0.90 (m, 3 H). LC-MS: t_{R} (min) 3.70 (20-100% ACN with 0.1% TFA 6 min), m/z [M + H]⁺ C₁₃H₁₅FN₃O₂ requires: 289.3, found: 290.1 HPLC t_{R} (min) 5.23, 98% (20-100% ACN with 0.1% TFA 10 min.) |
| 48 | (R)-1- cyclo- hexylethyl amine | 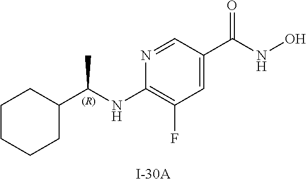 | ¹H NMR (400 MHz, MeOD-d4) δ 8.24 (s, 1H), 7.56 (s, 1 H), 7.53 (s, 1 H), 4.63 (s, 2 H), 4.09 (t, J = 6.72 Hz, 1 H), 1.88-1.65 (m, 5 H), 1.55-1.45 (m, 1 H), 1.31-1.22 (m, 3 H), 1.22-1.15 (m, 3 H), 1.10-0.90 (m, 2 H). LC-MS: t_{R} (min) 3.37 (20-100% ACN with 0.1% TFA 6 min), m/z [M + H]⁺ C₁₄H₂₀FN₃O₂ requires: 281.3, found: 282.2 HPLC t_{R} (min) 4.84, 97% (20-100% ACN with 0.1% TFA 10 min.) |
| 49 | (S)-1- cyclo- hexylethyl amine | 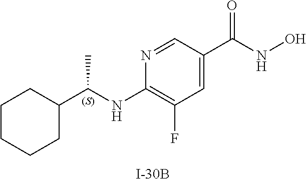 | ¹H NMR (400 MHz, MeOD-d4) δ 8.24 (s, 1H), 7.56 (s, 1 H), 7.53 (s, 1 H), 4.63 (s, 2 H), 4.09 (t, J = 6.97 Hz, 1 H), 1.88-1.65 (m, 5 H), 1.51 (s, 1 H), 1.31-1.20 (m, 3 H), 1.22-1.18 (m, 3 H), 1.10-0.90 (m, 2 H). LC-MS: t_{R} (min) 3.50 (20-100% ACN with 0.1% TFA 6 min), m/z [M + H]⁺ C₁₄H₂₀FN₃O₃ requires: 281.3, found: 282.1 HPLC t_{R} (min) 5.10, 98% (20-100% ACN with 0.1% TFA 10 min.) |
| 50 | (1S,2S)- 2-methoxy cyclo- pentylamine | 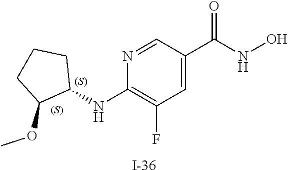 | ¹H NMR (400 MHz, CDCl3-d) δ 8.27 (s, 1 H), 7.58-7.39 (m, 1 H), 5.06 (br, s, 1 H), 4.31 (br, s, 1 H), 3.68 (br, s, 1 H), 3.34 (s, 3 H), 2.25-2.15 (m, 1 H), 1.93-1.88 (m, 1 H), 1.85-1.75 (m, 3 H), 1.50-1.40 (m, 1 H). LC-MS: t_{R} (min) 1.93 (20-100% ACN with 0.1% TFA 6 min), m/z [M + H]⁺ C₁₂H₁₆FN₃O₂ requires: 269.3, found: 270.1 HPLC t_{R} (min) 3.71, 97% (20-100% ACN with 0.1% TFA 10 min.) |
| 51 | (3S,4R)- 4-methoxy tetra- hydrofuran- 3- yl)amine | 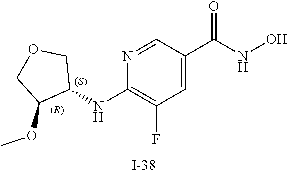 | ¹H NMR (400 MHz, MeOH-d4) δ 8.33 (s, 1 H), 7.60 (d, J = 11.98 Hz, 1 H), 4.60 (br, s, 1 H), 4.20-4.05 (m, 2 H), 4.00-3.90 (m, 1 H), 3.85-3.75 (m, 2 H), 3.48 (s, 3 H). LC-MS: t_{R} (min) 1.39 (20-100% ACN with 0.1% TFA 6 min), m/z [M + H]⁺ C₁₁H₁₄FN₃O₂ requires: 271.3, found: 272.1 HPLC t_{R} (min) 2.97, 97% (20-100% ACN with 0.1% TFA 10 min.) |
| 52 | 3,3- difluoro- 1- (methoxy- methyl) cyclobutan- 1-amine | 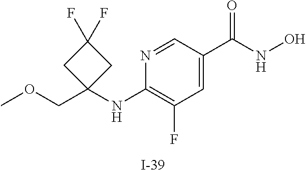 | ¹H NMR (400 MHz, MeOH-d4) δ 8.16 (s, 1 H), 7.48 (d, J = 12.2 Hz 1 H), 4.52 (s, 2 H), 3.63 (s, 2 H), 3.26-3.23 (m, 3 H), 2.82-2.72 (m, 4H). LC-MS: t_{R} (min) 3.00 (20-100% ACN with 0.1% TFA 6 min), m/z [M + H]⁺ C₁₂H₁₄F₃N₃O₃ requires: 305.3, found: 306.1 HPLC t_{R} (min) 5.02, 93% (20-100% ACN with 0.1% TFA 10 min.) |
| 53 | 3- ethyl- oxetan-3- amine | 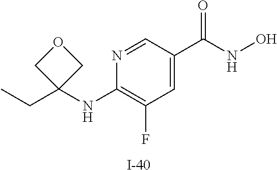 | ¹H NMR (400 MHz, MeOH-d4) δ 8.22 (s, 1 H), 7.59 (d, J = 11.98 Hz, 1 H), 4.80 (br, s, 2 H), 4.60 (br, s, 2 H), 2.22 (q, J = 6.93 Hz, 2 H), 0.92 (t, J = 6.95 Hz, 3 H). LC-MS: t_{R} (min) 1.10 (20-100% ACN with 0.1% TFA 6 min), m/z [M + H]⁺ C₁₁H₁₃FN₃O₃ requires: 255.2, found: 256.1 HPLC t_{R} (min) 3.80, 99% (20-100% ACN with 0.1% TFA 10 min.) |
| 54 | 1,1′- bi(cyclo- propan)- 1-amine | 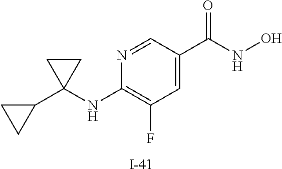 | ¹H NMR (400 MHz, MeOD-d4) δ 8.07 (s, 1 H), 7.32 (d, J = 11.98 Hz, 1 H), 4.40 (br, s, 1 H), 3.08 (s, 1 H), 1.35- 1.25 (m, 1 H), 0.48 (d, J = 5.38 Hz, 4 H), 0.38-0.42 (m, 2 H), 0.01 (s, 2 H). LC-MS: t_{R} (min) 1.20 (20-100% ACN with 0.1% TFA 6 min), m/z [M + H]⁺ C₁₁H₁₃FN₃O₃ requires: 251.3, found: 252.1 HPLC t_{R} (min) 3.17, 96% (20-100% ACN with 0.1% TFA 10 min.) |
| 55 | (S)-1- methyl- 2- oxo- pyrrolidin- 3-amine | 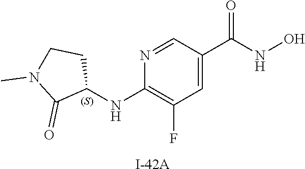 | ¹H NMR (400 MHz, DMSO-d6) δ 11.05 (br, s, 1 H), 9.00 (br, s, 1 H), 8.50 (br, s, 1 H), 7.65 (d, J = 12.0 Hz, 1 H), 7.45 (d, J = 8.80 Hz, 1 H), 4.90-4.80 (m, 1 H), 3.45- 3.40 (m, 2 H), 2.76 (s, 3 H), 2.30-2.20 (m, 1 H), 1.90- 2.00 (m, 1 H). LC-MS: t_{R} (min) 1.10 (20-100% ACN with 0.1% TFA 6 min), m/z [M + H]⁺ C₁₁H₁₃FN₃O₃ requires: 268.3, found: 269.1 HPLC t_{R} (min) 1.54, 96% (20-100% ACN with 0.1% TFA 10 min.) |
| 56 | (S)-2- oxo- pyrrolidin- 3-amine | 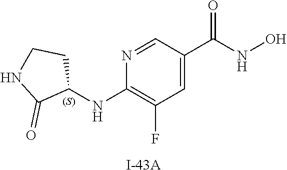 | ¹H NMR (400 MHz, DMSO-d6) δ 11.05 (br, s, 1 H), 8.95 (br s, 1 H), 8.25 (s, 1 H), 7.83 (s, 1 H), 7.65 (d, J = 12.0 Hz, 1 H), 7.45 (d, J = 8.80 Hz, 1 H), 4.70-4.85 (m, 1 H), 3.23 (d, J = 6.34 Hz, 2 H), 2.40-2.30 (m, 1 H), 2.15- 2.05 (m, 1 H). LC-MS: t_{R} (min) 1.10 (20-100% ACN with 0.1% TFA 6 min), m/z [M + H]⁺ C₁₂H₁₆FN₃O₃ requires: 254.2, found: 255.1 HPLC t_{R} (min) 1.71, 96% (20-100% ACN with 0.1% TFA 10 min.) |
| 57 | (R)-1- methyl- 2- oxo- pyrrolidin- 3-amine | 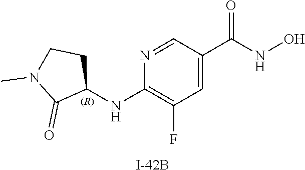 | ¹H NMR (400 MHz, DMSO-d6) δ 11.05 (br, s, 1 H), 8.99 (br, s, 1 H), 8.25 (s, 1 H), 7.65 (d, J = 12.2 Hz, 1 H), 7.42 (d, d, J = 8.07 Hz, 1 H), 4.82 (d, J = 9.05 Hz, 1 H), 3.34-3.27 (m, 2 H), 2.76 (s, 3 H), 2.40-2.26 (m, 1 H), 2.03-1.92 (m, 1 H). LC-MS: t_{R} (min) 1.20 (20-100% ACN with 0.1% TFA 6 min), m/z [M + H]⁺ C₁₂H₁₆FN₃O3 requires: 268.3, found: 269.1 HPLC t_{R} (min) 1.75, 99% (20-100% ACN with 0.1% TFA 10 min.) |
| 58 | (R)-2- oxo- pyrrolidin- 3-amine | 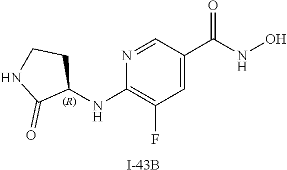 | ¹H NMR (400 MHz, DMSO-d6) δ 11.0 (br, s, 1 H), 9.05 (br, s, 1 H), 8.45 (s, 1 H), 7.84 (s, 1 H), 7.65 (d, J = 12.0 Hz, 1 H), 7.34 (d, J = 8.80 Hz, 1 H), 4.77 (d, J = 9.29 Hz, 1 H), 3.18-3.28 (m, 2 H), 2.30-2.40 (m, 1 H), 2.15-2.05 (m, 1 H). LC-MS: tR (min) 1.15 (20-100% ACN with 0.1% TFA 6 min), m/z [M + H]⁺ C₁₂H₁₆FN₃O₃ requires: 254.2, found: 255.1 HPLC tR (min) 1.55, 93% (20-100% ACN with 0.1% TFA 10 min.) |
| 59 | 1-(2- methoxy ethyl) cyclopropyl) amine | 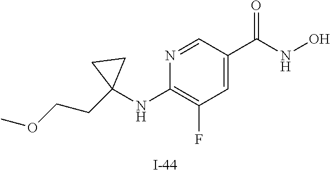 | ¹H NMR (400 MHz, MeOD-d4) δ 8.32 (s, 1 H), 7.57 (d, J = 11.98 Hz, 1 H), 3.55 (t, J = 6.85, 2 H), 3.30 (s, 3 H), 1.96 (t, J = 6.85 Hz, 2 H), 0.80 (s, 4 H). LC-MS: t_{R} (min) 1.20 (20-100% ACN with 0.1% TFA 6 min), m/z [M + H]⁺ C₁₂H₁₆FN₃O₃ requires: 269.3, found: 270.1 HPLC t_{R} (min), 2.07, 99% (20-100% ACN with 0.1% TFA 10 min.) |
| 60 | 3- phenyl- oxetan-3- amine | 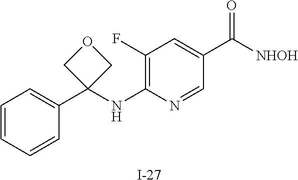 | ¹H NMR (400 MHz, DMSO-d6) δ 10.95 (br s, 1 H), 8.97 (s, 1 H), 8.38 (m, 1 H), 8.02 (m, 1 H), 7.69 (m, 1 H), 7.53 (m, 2 H), 7.34 (m, 2 H), 7.24 (m, 1 H), 4.99 (m, 2 H), 4.78 (m, 2 H). LC-MS: m/z [M + H]⁺ C₁₅H₁₄FN₃O₃ requires: 303.2, found: 304.1 HPLC t_{R} (min), 4.81, 95% (10-100% ACN with 0.1% TFA 10 min.) |
| 61 | 3- (pyridin- 2-yl) oxetan-3- amine | 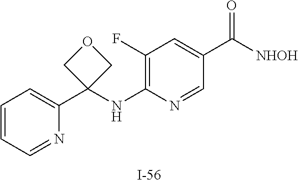 | ¹H NMR (400 MHz, DMSO-d6) δ 10.97 (br s, 1 H), 8.97 (s, 1 H), 8.63 (m, 1 H), 8.46 (m, 1 H), 7.99 (m, 1 H), 7.71-7.66 (m, 2 H), 7.31-7.15 (m, 2 H), 5.01 (m, 2 H), 4.87 (m, 2 H). LC-MS: m/z [M + H]⁺ C₁₄H₁₃FN₄O₃ requires: 304.2, found: 305.1 HPLC t_{R} (min) 1.27, 93% (10-100% ACN with 0.1% TFA 10 min.) |

### Examples 62-74

In the following examples (62-74), the compounds were synthesized by following the same experimental procedure as described in Example 30 with the listed amine and 5-bromo-2-(bromomethyl)-3-fluoropyridine as the starting materials for step 4.

| Ex. | Amine | Compound | Characterization Data |
|---|---|---|---|
| 62 | 1H- pyrrolo[2,3- b]pyridine | 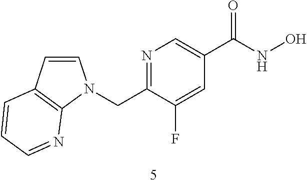 | ¹H NMR (400 MHz, DMSO-d6) δ 11.41 (br s, 1 H), 9.31 (s, 1 H), 8.57 (m, 1 H), 8.18 (m, 1 H), 7.98 (m, 2 H), 7.60 (m, 1 H), 7.08 (m, 1 H), 6.52 (m, 1 H), 5.71 (s, 2 H). LC-MS: m/z [M + H]⁺ C₁₄H₁₁FN₄O₂ requires: 286.2, found: 287.1 HPLC t_{R} (min) 3.21, 98% (20-100% ACN with 0.1% TFA 10 min.) |
| 63 | 2- difluoro methyl)- 1H- benzo[d] imidazole | 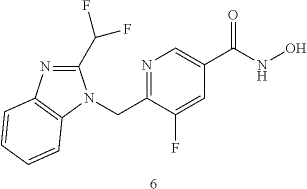 | ¹H NMR (400 MHz, DMSO-d6) δ 11.41 (br s, 1 H), 9.32 (s, 1 H), 8.51 (s, 1 H), 8.01 (m, 1 H), 7.63 (m, 1 H), 7.62 (m, 1 H), 7.53-7.27 (m, 3 H), 5.92 (s, 2 H). LC-MS: m/z [M + H]⁺ C₁₅H₁₁F₃N₄O₂ requires: 336.21, found: 337.1 HPLC t_{R} (min) 4.87, 97% (10-100% ACN with 0.1% TFA 10 min.) |
| 64 | 2,2- dimethyl- 2H- pyrido[3,2- b][1,4] oxazin- 3(4H)- one | 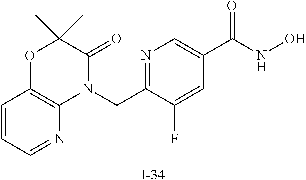 | ¹H NMR (400 MHz, DMSO-d6) δ 11.37 (br s, 1 H), 9.30 (s, 1 H), 8.52 (s, 1 H), 7.97 (m, 1 H), 7.88 (m, 1 H), 7.44 (m, 1 H), 7.03 (m, 1 H), 5.42 (s, 2 H), 1.51 (s, 6 H). LC-MS: m/z [M + H]⁺ C₁₆H₁₅FN₄O₄ requires: 346.3, found: 347.1 HPLC t_{R} (min) 5.04, 100% (10-100% ACN with 0.1% TFA 10 min.) |
| 65 | 3- methyl- 3,4- dihydro quinazolin- 2(1H)- one | 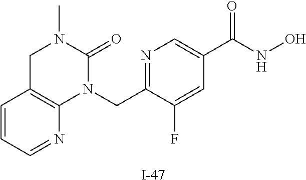 | ¹H NMR (400 MHz, DMSO-d6) δ 11.40 (br s, 1 H), 9.30 (s, 1 H), 8.55 (s, 1 H), 7.94 (m, 1 H), 7.15-7.11 (m, 2 H), 6.93 (m, 1 H), 6.74 (m, 1 H), 5.22 (s, 2 H), 4.40 (s, 2 H), 2.90 (s, 3 H). LC-MS: m/z [M + H]⁺ C₁₆H₁₅FN₄O₃ requires: 330.3, found: 331.1 HPLC t_{R} (min) 4.93, 100% (10-100% ACN with 0.1% TFA 10 min.) |
| 66 | 3,4- dihydro- 2H- thieno[3, 2-b] indole 1,1- dioxide | 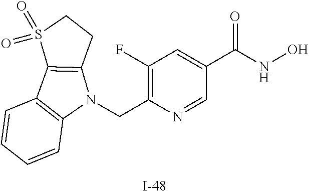 | ¹H NMR (400 MHz, DMSO-d6) δ 8.62 (s, 1 H), 8.02 (m, 1 H), 7.56 (m, 2 H), 7.27 (m, 2 H), 5.68 (s, 2 H), 3.94 (m, 2 H), 3.46 (m, 2 H). LC-MS: m/z [M + H]⁺ C₁₇H₁₄FN₃O₄S requires: 375.3, found: 376.1 HPLC t_{R} (min) 4.85, 96% (10-100% ACN with 0.1% TFA 10 min.) |
| 67 | 2- (trifluoro- methyl)- 1H- benzo[d] imidazole | 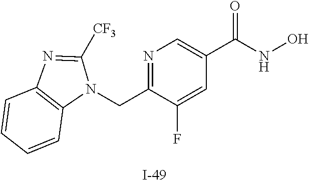 | ¹H NMR (400 MHz, DMSO-d6) δ 11.41 (br s, 1 H), 9.33 (s, 1 H), 8.49 (s, 1 H), 8.04 (m, 1 H), 7.84 (m, 1 H), 7.72 (m, 1 H), 7.44 (m, 2 H), 5.95 (s, 2 H). LC-MS: m/z [M + H]⁺ C₁₅H₁₀F₄N₄O₂ requires: 354.2, found: 355.1 HPLC t_{R} (min) 4.94, 98% (10-100% ACN with 0.1% TFA 10 min.) |
| 68 | 2- methyl- 1H- pyrrolo 2,3- b]pyridine | 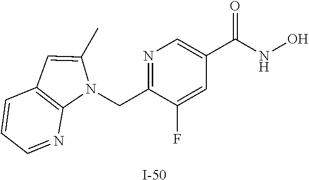 | ¹H NMR (400 MHz, DMSO-d6) δ 11.39 (br s, 1 H), 9.31 (s, 1 H), 8.51 (s, 1 H), 8.06 (m, 1 H), 7.97 (m, 1 H), 7.84 (m, 1 H), 7.03 (m, 1 H), 6.29 (s, 1 H), 5.69 (s, 2 H), 2.40 (s, 3 H). LC-MS: m/z [M + H]⁺ C₁₅H₁₃FN₄O₂ requires: 300.2, found: 301.1 HPLC t_{R} (min) 3.83, 100% (20-100% ACN with 0.1% TFA 10 min.) |
| 69 | 2,3- dimethyl- 1H- pyrrolo[2,3- b]pyridine | 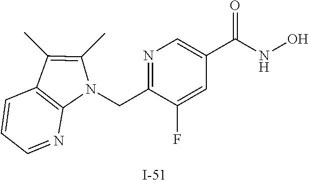 | ¹H NMR (400 MHz, DMSO-d6) δ 11.39 (br s, 1 H), 9.31 (s, 1 H), 8.51 (s, 1 H), 8.05 (m, 1 H), 7.95 (m, 1 H), 7.81 (m, 1 H), 7.01 (m, 1 H), 5.67 (s, 2 H), 2.31 (s, 3 H), 2.21 (s, 3 H). LC-MS: m/z [M + H]⁺ C₁₆H₁₅FN₄O₂ requires: 314.3, found: 315.1 HPLC t_{R} (min) 4.17, 100% (20-100% ACN with 0.1% TFA 10 min.) |
| 70 | oxazolo [4,5- b] pyridin- 2(3H)- one | 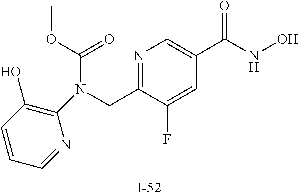 | ¹H NMR (400 MHz, CD₃OD-d₄) δ 8.70 (s, 1 H), 7.98 (m, 2 H), 7.47 (m, 1 H), 7.38 (m, 1 H), 5.06 (s, 2 H), 3.64 (s, 3 H). LC-MS: m/z [M + H]⁺ C₁₄H₁₃FN₄O₅ requires: 336.2, found: 337.1 HPLC t_{R} (min) 4.18, 96% (10-100% ACN with 0.1% TFA 10 min.) |
| 71 | 2- methyl- 3H- imidazo [4,5-b] pyridine | 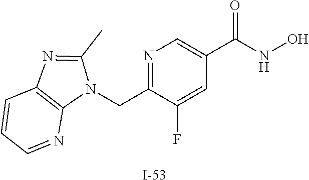 | ¹H NMR (400 MHz, DMSO-d6) δ 11.30 (br s, 1 H), 9.27 (s, 1 H), 8.47 (s, 1 H), 8.12 (m, 1 H), 7.95 (m, 1 H), 7.88 (m, 1 H), 7.15 (m, 1 H), 5.68 (s, 2 H), 2.44 (s, 3 H). LC-MS: m/z [M + H]⁺ C₁₄H₁₂FN₅O₂ requires: 301.2, found: 302.1 HPLC t_{R} (min) 3.60, 98% (10-100% ACN with 0.1% TFA 10 min.) |
| 72 | 4-(1H- benzo[d] imidazol- 2- yl) morpholine | 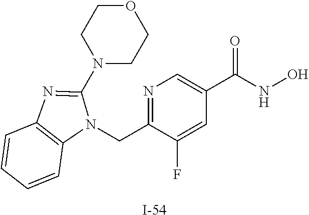 | ¹H NMR (400 MHz, DMSO-d6) δ 11.45 (br s, 1 H), 9.34 (s, 1 H), 8.59 (s, 1 H), 8.03 (m, 1 H), 7.45 (m, 1 H), 7.15- 7.03 (m, 3 H), 5.55 (s, 2 H), 3.67 (m, 4 H), 3.14 (m, 4 H). LC-MS: m/z [M + H]⁺ C₁₈H₁₈FN₅O₃ requires: 371.3, found: 372.1 HPLC t_{R} (min) 3.66, 95% (10-100% ACN with 0.1% TFA 10 min.) |
| 73 | 2- methyl- 2,3,4,5- tetra- hydro- 1H- pyrido[4,3-b] indole | 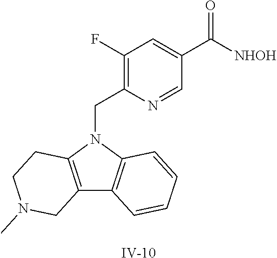 | ¹H NMR (400 MHz, DMSO-d6) δ 11.40 (br s, 1 H), 9.36 (s, 1 H), 8.61 (s, 1 H), 7.95 (m, 1 H), 7.35 (m, 2 H), 7.03- 6.94 (m, 2 H), 5.50 (s, 2 H), 3.52 (s, 2 H), 2.83 (m, 2 H), 2.72 (m, 2 H), 2.42 (s, 3 H). LC-MS: m/z [M + H]⁺ C₁₉H₁₉FN₄O₂ requires: 354.3, found: 355.2 HPLC t_{R} (min) 4.58, 99% (10-100% ACN with 0.1% TFA 10 min.) |
| 74 | 1,3,4,5- tetra- hydro- pyrano [4,3-b] indole | 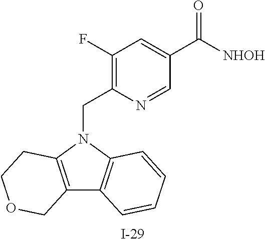 | ¹H NMR (400 MHz, DMSO-d6) δ 11.41 (br s, 1 H), 9.35 (s, 1 H), 8.61 (s, 1 H), 8.01 (m, 1 H), 7.40-7.32 (m, 2 H), 7.06-6.95 (m, 2 H), 5.54 (s, 2 H), 4.78 (s, 2 H), 3.95 (m, 2 H), 2.84 (m, 2 H). LC-MS: m/z [M + H]⁺ C₁₈H₁₆FN₃O₃ requires: 341.3, found: 342.1 HPLC t_{R} (min) 4.99, 94% (10-100% ACN with 0.1% TFA 10 min.) |

### Examples 75-80: Characterization of Compounds

| Ex. | Compound | Characterization |
|---|---|---|
| 75 | 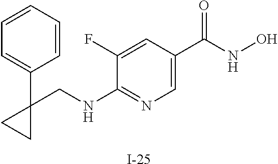 | ¹H NMR (400 MHz, Solvent) δ ppm 8.18 (s, 1 H) 7.50 (br d, J = 11.98 Hz, 1 H) 7.31-7.38 (m, 2 H) 7.23 (br t, J = 7.46 Hz, 2 H) 7.10-7.17 (m, 1 H) 3.74 (s, 2 H) 0.93-0.99 (m, 2 H) 0.80-0.86 (m, 2 H) |
| 76 | 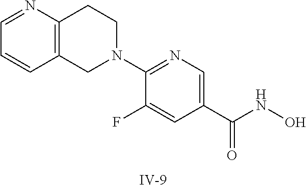 [SMILES: O=C(NO)c1cnc(N2CCc3ncccc3C2)c(F)c1 ; "IV-9"] | ¹H NMR (400 MHz, Solvent) δ ppm 8.39 (s, 1 H) 8.35 (d, J = 4.65 Hz, 1 H) 7.64-7.76 (m, 2 H) 7.27 (dd, J = 7.58, 5.14 Hz, 1 H) 4.86 (s, 2 H) 4.03 (t, J = 5.87 Hz, 2 H) 3.10 (t, J = 5.87 Hz, 2 H) |
| 77 | 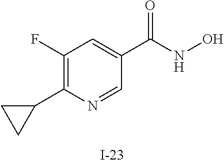 | ¹H NMR (400 MHz, METHANOL-d4) δ ppm 8.57 (s, 1 H) 7.77 (d, J = 10.27 Hz, 1 H) 2.31-2.46 (m, 1 H) 1.08-1.16 (m, 4 H) LCMS R_{T} = 2.79 min, m/z = 197.1 [M + H]⁺. |
| 78 | 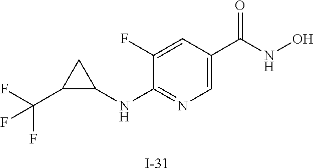 | ¹H NMR (400 MHz, METHANOL-d4) δ ppm 8.34 (s, 1 H), 7.60 (br d, J = 11.49 Hz, 1 H) 3.22-3.28 (m, 1 H) 1.82-1.95 (m, 1 H) 1.21-1.31 (m, 1 H) 1.16 (br dd, J = 10.03, 4.89 Hz, 1 H) |
| 79 | 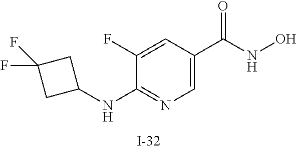 | ¹H NMR (400 MHz, METHANOL-d4) δ ppm 8.30 (s, 1 H) 7.61 (br d, J = 11.4 Hz, 1 H) 4.32-4.47 (m, 1 H) 2.94-3.12 (m, 2 H) 2.54-2.76 (m, 2 H) |
| 80 | 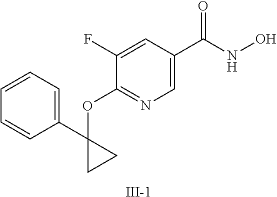 | ¹H NMR (400 MHz, METHANOL-d4) δ ppm 8.21 (s, 1 H) 7.82 (br d, J = 10.27 Hz, 1 H) 7.25-7.32 (m, 4 H) 7.15-7.23 (m, 1 H) 1.44-1.50 (m, 2 H) 1.36-1.43 (m, 2 H) |

### Example 81

Biochemical Assay The compounds disclosed herein were tested for potency, against HDAC6 and selectivity against HDAC1 in a biochemical assay. A biochemical assay was adopted using a luminescent HDAC-Glo I/II assay (Promega) and measured the relative activity of HDAC6 and HDAC1 recombinant proteins. Compounds were first incubated in the presence of HDAC6 or HDAC1 separately, followed by addition of the luminescent substrate. The data was acquired using a plate reader and the biochemical IC₅₀ were calculated from the data accordingly. Data is tabulated in Table 2. From these studies, it was determined that the compounds of the present disclosure are selective inhibitors of HDAC6 over HDAC1, providing selectivity ratios from about 5 to about 30,0000.

TABLE 2
Evaluation of HDAC6 Activity and Selectivity for Disclosed Compounds.

| Compound | HDAC6 | Compound | HDAC6 |
|---|---|---|---|
| ID | IC50 (nM) | ID | IC50 (nM) |
| 1 | 136 | I-7A | 1.5 |
| I-1 | 12.5 | I-7B | 4.13 |
| I-6 | 1.61 | I-2 | 104 |
| I-4 | 16.7 | I-10 | 76.6 |
| I-5 | 6.68 | I-3 | 73 |
| IV-1 | 8.26 | I-8A | 1.65 |
| IV-4 | 1.8 | 5 | 1.52 |
| I-9A | 0.351 | I-26A | 4.96 |
| IV-2 | 0.677 | I-26B | 2.33 |
| IV-3 | 3.35 | 6 | 2.54 |
| I-9B | 0.791 | I-27 | 0.992 |
| I-11 | 0.639 | I-28A | 1.77 |
| I-19 | 0.425 | I-29 | 13.3 |
| I-18 | 1.68 | I-30A | 1.31 |
| I-16 | 1.61 | I-30B | 7.93 |
| I-8B | 0.275 | I-31 | 13.8 |
| I-13 | 73 | I-32 | 13 |
| I- 14 | 28.3 | I-33 | 29.4 |
| I-17 | 1.12 | I-34 | 2.28 |
| 4 | 1.2 | I-35 | 23 |
| I-25 | 0.669 | I-36 | 5.31 |
| IV-9 | 0.595 | I-37 | 2.18 |
| I-21 | 0.601 | I-38 | 5.67 |
| I-22 | 3.36 | I-39 | 1.12 |
| I-23 | 1.59 | I-40 | 3.36 |
| III-1 | 1.79 | I-41 | 3.97 |
| IV-5 | 2.04 | I-42A | 34.3 |
| I-12B | 0.809 | I-42B | 41.7 |
| IV-10 | 2.4 | I-43A | 51.1 |
| IV-7 | 1.1 | I-43B | 25.7 |
| IV-6 | 4.06 | I-44 | 4.71 |
| IV-8 | 10.3 | I-45 | 6.51 |
| I-15 | 2.64 | III-2 | 14.6 |
| I-20 | 3.78 | I-46 | 29.2 |
| I-47 | 4.36 | I-52 | 4.29 |
| I-48 | 2.27 | I-53 | 8.69 |
| I-49 | 5.11 | I-54 | 80.3 |
| I-50 | 2.98 | I-55 | 3.88 |
| I-51 | 1.94 | I-56 | 7.67 |

### EMBODIMENTS

1. A compound of Formula (I), or pharmaceutically acceptable salt thereof:

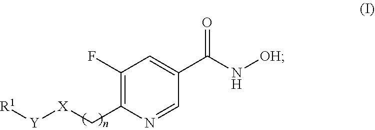

wherein

n is 0 or 1;

X is O, NR⁴, or CR⁴R⁴′;

Y is a bond, CR²R³ or S(O)₂;

R¹ is selected from the group consisting of H, amido, carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R¹ and R² taken together with the carbon atom to which they are attached form a carbocyclyl or heterocyclyl; or

R² and R³ taken together with the carbon atom to which they are attached form a carbocyclyl or heterocyclyl; and

R⁴ and R⁴′ are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂) aryl, and —(CH₂)-heteroaryl, or

R⁴ and R⁴′ taken together with the carbon atom to which they are attached form a carbocyclyl or heterocyclyl;

wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃ morpholine, and —C(O)N-cyclopropyl.

2. The compound of embodiment 1, wherein n is 1.
3. The compound of embodiment 1, wherein n is 0.
4. The compound of any one of embodiments 1-3, wherein X is NR⁴ or CR⁴R⁴′.
5. The compound of any one of embodiments 1-4, wherein Y is CR²R³.
6. The compound of any one of embodiments 1-4, wherein X is NR⁴ and Y is S(O)₂.
7. The compound of any one of embodiments 1-6, wherein R¹ is a heteroaryl selected from the group consisting of pyrimidinyl, pyridinyl, pyridazine, and pyrazine.
8. The compound of any one of embodiments 1-7, wherein R¹ is pyridinyl.
9. The compound of any one of embodiments 1-6, wherein R¹ is phenyl.
10. The compound of any one of embodiments 1-5, wherein X is CR⁴R⁴′, Y is a bond, and R¹ is H.
11. The compound of any one of embodiments 1-6, wherein R¹ and R² taken together with the carbon atom to which they are attached form a C₃₋₁₂ carbocyclyl.
12. The compound of any one of embodiments 1-10, wherein R² and R³ are independently selected from the group consisting of H, F, C₁₋₆ alkyl, C₃₋₆ cycloalkyl, —(CH₂)—C₃₋₆ cycloalkyl, 4- to 6-membered heterocyclyl, and —(CH₂) (4- to 6-membered heterocyclyl).
13. The compound of any one of embodiments 1-10, wherein R² and R³ taken together with the carbon atom to which they are attached form a C₃₋₆ cycloalkyl.
14. The compound of any one of embodiments 1-10, wherein R² and R³ taken together with the carbon atom to which they are attached form a cyclopropyl.
15. The compound of any one of embodiments 1-10, wherein and R³ taken together the carbon atom to which they are attached form a 4- to 6-membered heterocyclyl.
16. The compound of any one of embodiments 1-10, wherein R² is and R³ is C₃₋₆ cycloalkyl.
17. The compound of any one of embodiments 1-10, wherein R² is H and R³ is cyclopropyl.
18. The compound of any one of embodiments 1-10, wherein R² and R³ are C₁₋₆ alkyl.
19. The compound of any one of embodiments 1-10, wherein R² and R³ are methyl.
20. The compound of any one of embodiments 1-10, wherein R¹ is C₃₋₆ cycloalkyl or aryl, R² is H, and R³ is C₃₋₆ cycloalkyl or aryl.
21. The compound of any one of embodiments 1-20, wherein R⁴ is selected from the group consisting of H, alkyl, carbocyclyl, heterocyclyl, —(CH₂)-carbocyclyl, and —(CH₂)-heterocyclyl.
22. The compound of any one of embodiments 1-21, wherein R⁴ is H.
23. The compound of any one of embodiments 1-21, wherein R⁴ is —(CH₂)-heterocyclyl.
24. The compound of any one of embodiments 1-21, wherein R⁴ is —(CH₂)-oxetane.
25. The compound of any one of embodiments 1-21, wherein R⁴ is alkyl.
26. The compound of any one of embodiments 1-21, wherein R⁴ is methyl.
27, The compound of any one of embodiments 1-21, wherein R⁴ and R⁴′ are each H.
28, The compound of any one of embodiments 1-21, wherein R⁴ and R⁴′ are each alkyl.
29. The compound of any one of embodiments 1-21, wherein R⁴ and R⁴′ are each methyl.
30. The compound of any one of embodiments 1-21, wherein R⁴ and R⁴′ taken together with the carbon atom to which they are attached form a C₃₋₆ cycloalkyl.
31. The compound of any one of embodiments 1-21, wherein R⁴ and R⁴′ taken together with the carbon atom to which they are attached form a cyclopropyl.
32. The compound of embodiment 1, wherein the compound is a compound of Formula (Iasi

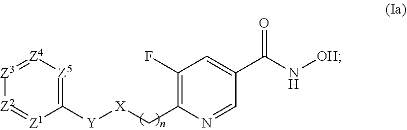

wherein
Z¹, Z², Z³, Z⁴, Z⁵ are independently selected from N and CR⁵;
wherein R⁵ is independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.
33. The compound of embodiment 32, wherein X is NR⁴ or CR⁴R^{4′.}
34. The compound of embodiment 32 or 33, wherein X is NR¹ and Y is CR²R³.
35. The compound of any one of embodiments 32-33, wherein X is —NR⁴ and Y is S(O)₂.
36. The compound of embodiment 32, wherein Z¹, Z², Z³, Z⁴ and Z⁵ are CR⁵.
37. The compound of embodiment 32, wherein Z¹ is N and Z², Z³, Z⁴ and Z⁵ are CR⁵.
38. The compound of embodiment 32, wherein Z² is N and Z¹, Z³, Z⁴ and Z⁵ are CR⁵.
39. The compound of embodiment 32, wherein Z³ is N and Z¹, Z², Z⁴ and Z⁵ are CR⁵.
40. The compound of any one of embodiments 32-39, wherein n is 1.
41. The compound of any one of embodiments 32-39, wherein n is 0.
42. The compound of any one of embodiments 32-41, wherein R² and R³ are independently selected from the group consisting of H, F, C₁₋₆ alkyl, C₃₋₆ cycloalkyl, —(CH₂)—C₃₋₆ cycloalkyl, 4- to 6-membered heterocyclyl, and —(CH₂)-(4- to 6-membered heterocyclyl).
43. The compound of any one of embodiments 32-41, wherein R² and R³ taken together with the carbon atom to which they are attached form a C₃₋₆ cycloalkyl.
44. The compound of any one of embodiments 32-41, wherein R² and R³ taken together with the carbon atom to which they are attached form a cyclopropyl.
45. The compound of any one of embodiments 32-41, wherein R² and R³ taken together with the carbon atom to which they are attached form a 4- to 6-membered heterocyclyl.
46. The compound of any one of embodiments 32-41, wherein R² is H and R³ is C₃₋₆ cycloalkyl.
47. The compound of any one of embodiments 32-41, wherein R² is H and R³ is cyclopropyl.
48. The compound of any one of embodiments 32-41, wherein R² and R³ are C₁₋₆ alkyl.
49. The compound of any one of embodiments 32-41, wherein R² and R³ are methyl.
50. The compound of any one of embodiments 32-49, wherein R⁴ is selected from the group consisting of H, alkyl, carbocyclyl, heterocyclyl, —(CH₂)-carbocyclyl, and —(CH₂)-heterocyclyl.
51. The compound of any one of embodiments 32-50, wherein R⁴ is H.
52. The compound of any one of embodiments 32-50, wherein R⁴ is —CH₂)-heterocyclyl.
53. The compound of any one of embodiments 32-50, wherein R⁴ is —(CH₂)-oxetane.
54. The compound of any one of embodiments 32-50, wherein —R⁴ is alkyl.
55. The compound of any one of embodiments 32-50, wherein R⁴ is methyl.
56. The compound of any one of embodiments 32-55, wherein R⁵ is independently selected from H and halogen.
57. The compound of any one of embodiments 32-55, wherein R⁵ is independently selected from H and fluoro.
58. The compound of any one of embodiments 32-35 and 40-57, wherein the compound is a compound of Formula (Ib)

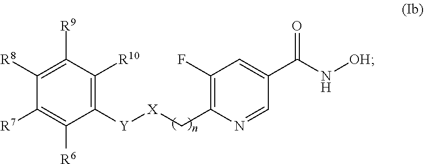

wherein
R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.
59. The compound of embodiment 58, wherein X is NR¹ or CR⁴R^{4′.}
60. The compound of embodiment 58 or 59, wherein X is NR⁴.
61. The compound of any one of embodiments 58-60, wherein Y is CR²R³.
62. The compound of any one of embodiments 58-60, wherein Y is S(O)₂.
63. The compound of any one of embodiments 58-6:2, wherein R⁶, R⁷, R⁸, R⁹, and R¹⁰, are independently selected from the group consisting of H and halogen.
64. The compound of any one of embodiments 58-62, wherein R⁶ and R¹⁰ are halogen and R⁷ R⁸, and R⁹ are H.
65. The compound of any one of embodiments 58-62, wherein R⁶ and R¹⁰ are fluoro and R⁷ R⁸, and R⁹ are H.

66. The compound of any one of embodiments 32-35 and 40-57, wherein the compound of Formula (I) is a compound of Formula (Ic):

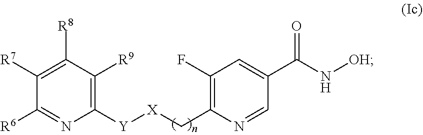

wherein
R⁶, R⁷, R⁸, and R⁹ are independently selected from the group consisting of H, halogen, alkyl, haloalkyl, carbocyclyl, heterocyclyl, aryl, heteroaryl, —CO₂H, CO₂-alkyl, —O-alkyl, —O-haloalkyl, —O-aryl, —O-heteroaryl, —SO₂-alkyl, and —CN.
67. The compound of embodiment 66, wherein X is NR⁴ or CR⁴R⁴′.
68. The compound of embodiment 66 or 67, wherein X is NR⁴.
69. The compound of any one of embodiments 66-68, wherein Y is CR²R³.
70. The compound of any one of embodiments 66-68, wherein Y is S(O)₂.
71. The compound of embodiment 1, wherein the compound is selected from the group consisting of:

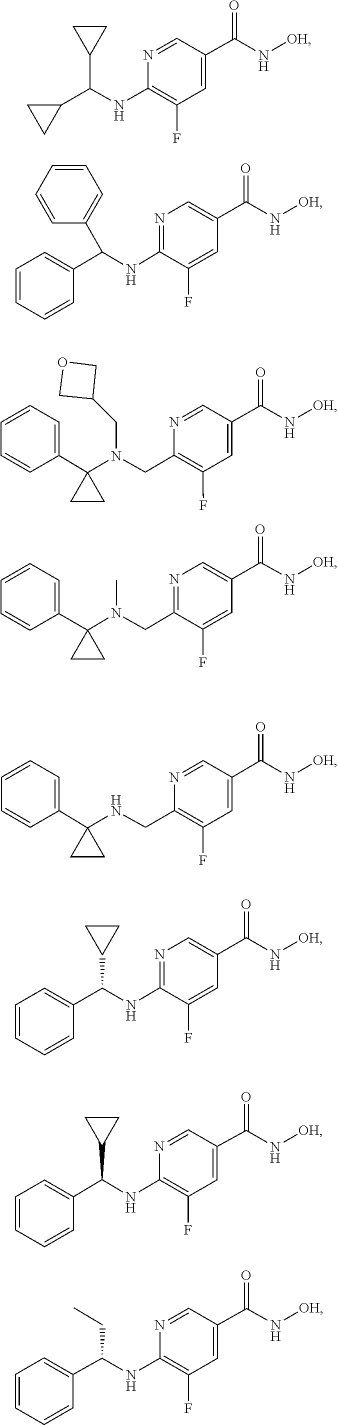

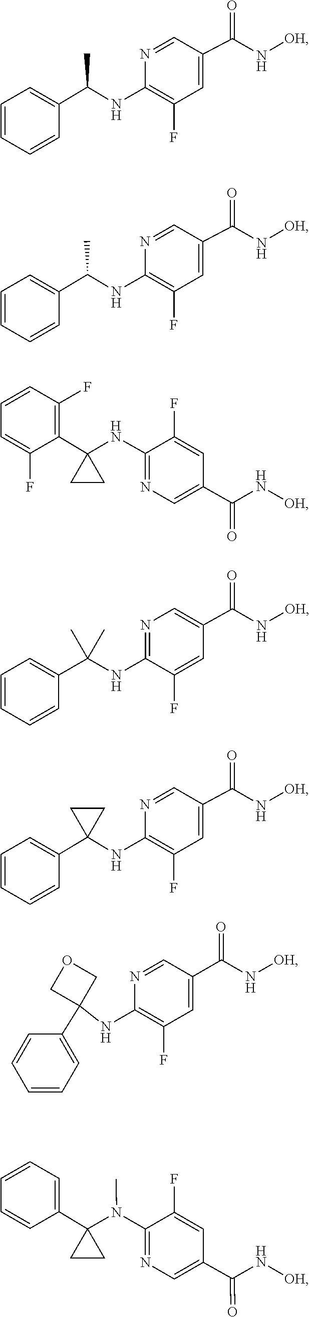

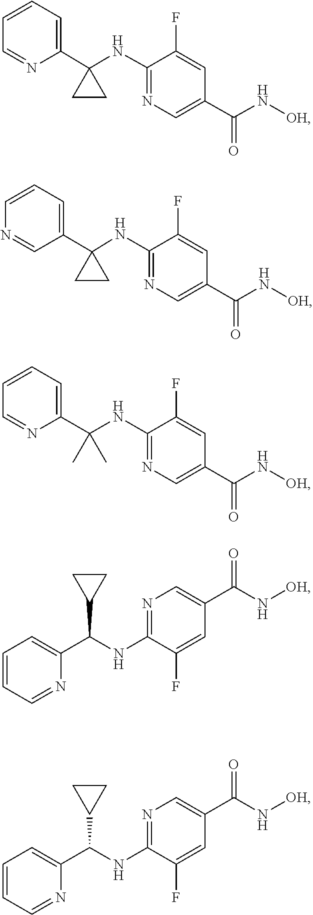

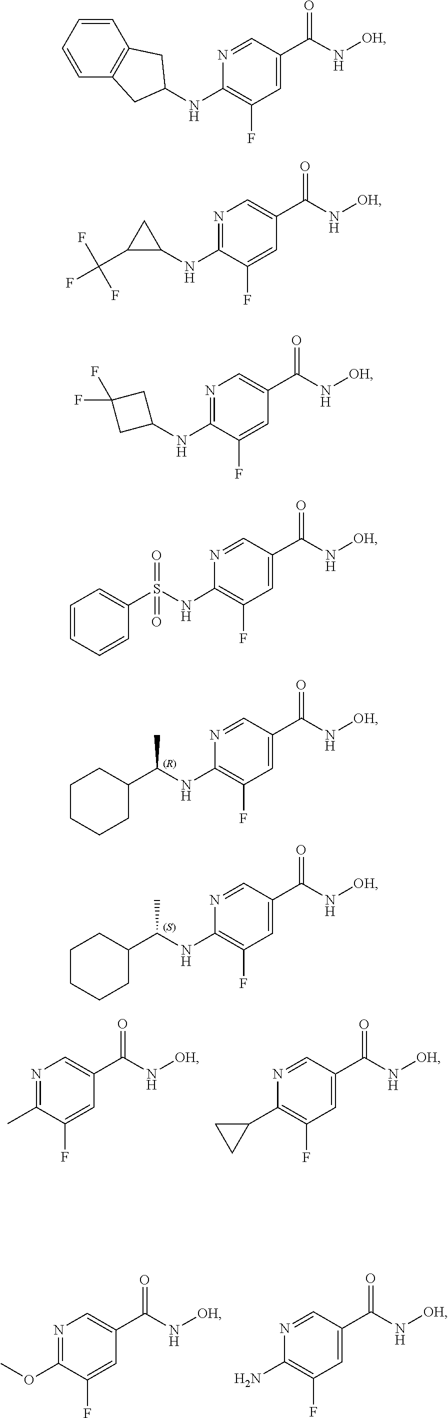

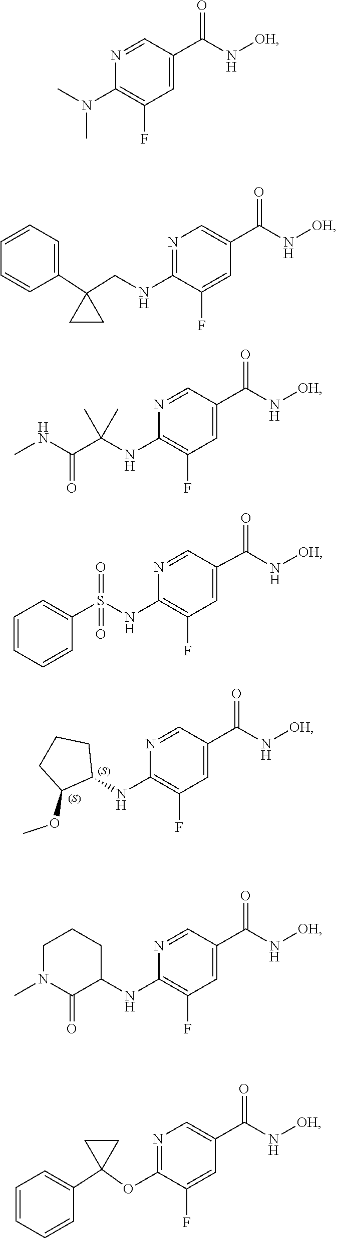

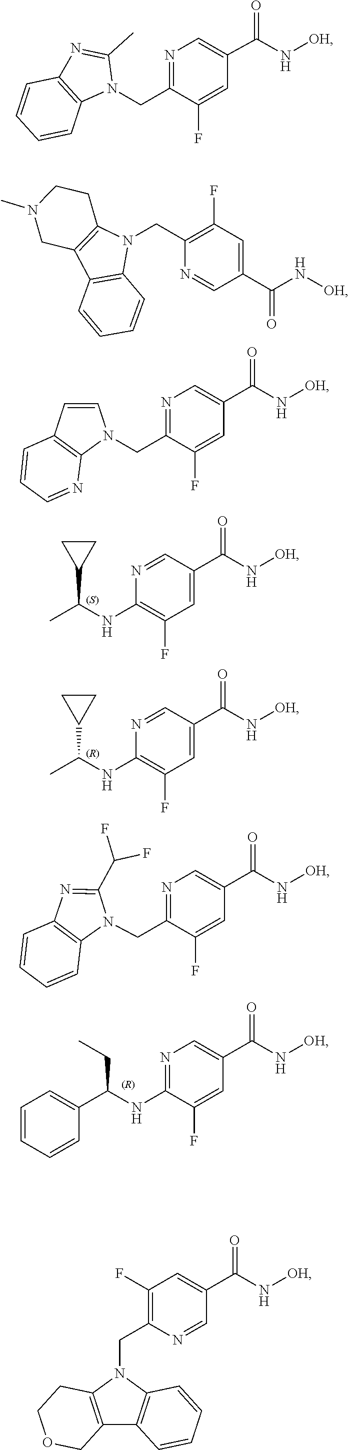

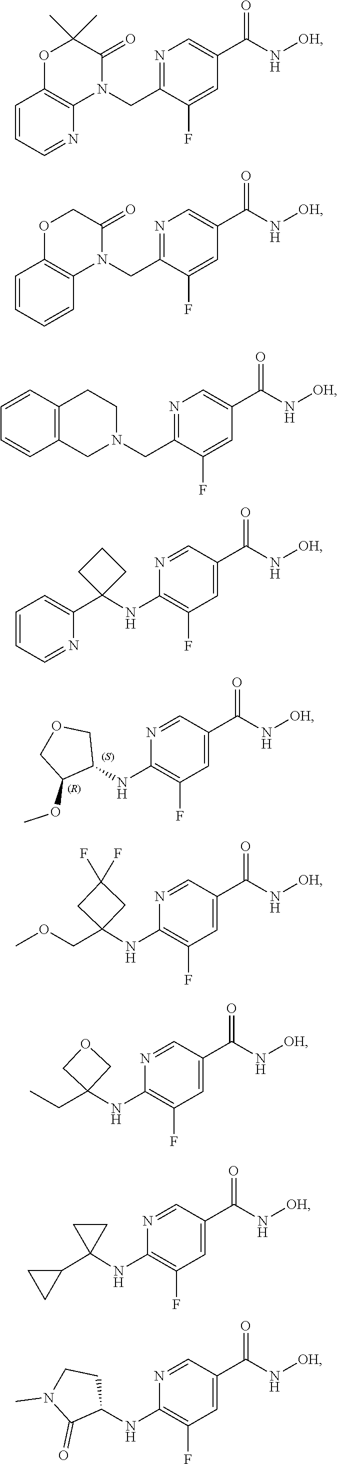

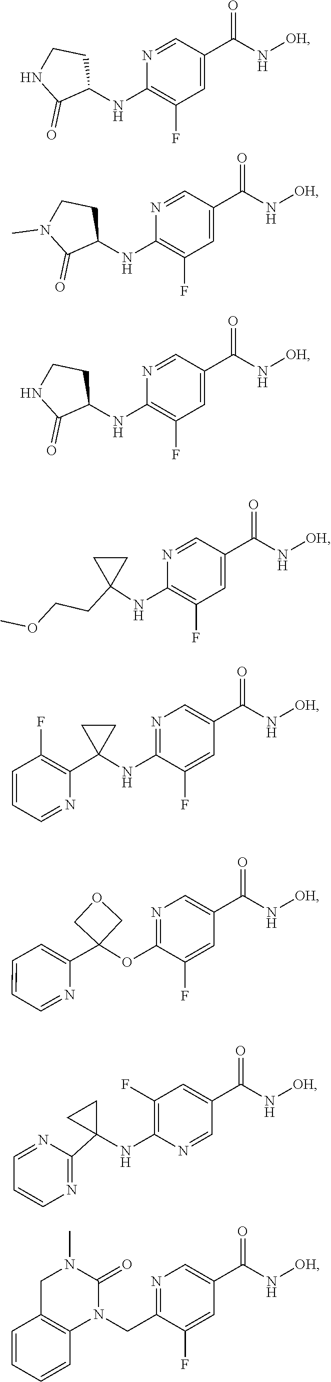

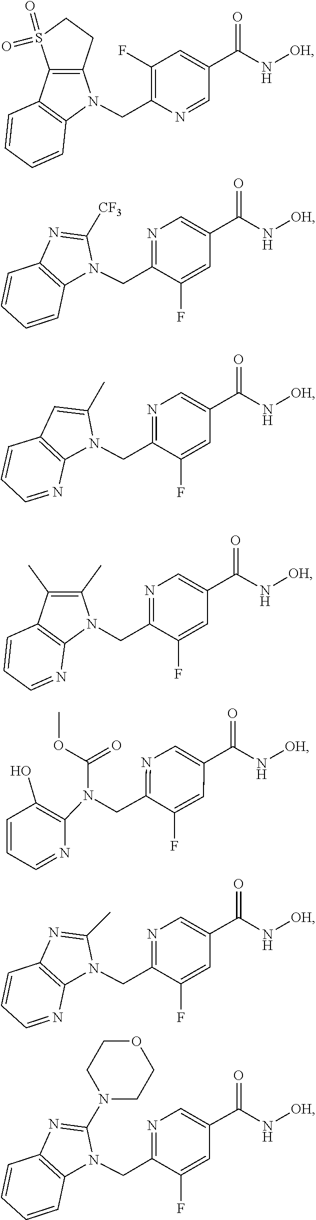

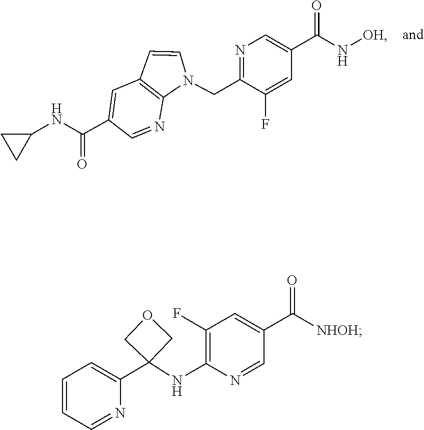

or a pharmaceutically acceptable salt thereof.
72. The compound of embodiment 1, wherein

n is 0 or 1;

X is NR⁴ or CR⁴R⁴′;

Y is CR²R³ or S(O)₂;

R¹ is selected from the group consisting of carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂) carbocyclyl, —(CH₂)-heterocyclyl, —(CH₂)-aryl, and —(CH₂)-heteroaryl, or

R² and R³ taken together with the carbon atom to which they are attached form a carbocyclyl or heterocyclyl, and

R⁴ and R⁴′ are independently selected from the group consisting of H, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or

R⁴ and R⁴′ taken together with the carbon atom to which they are attached form a carbocyclyl or heterocyclyl; and

wherein each alkyl, carbocyclyl, heterocyclyl, aryl, and heteroaryl is independently, optionally substituted with one or more substituents selected from the group consistinc, of halogen, oxo, hydroxy, alkoxy, —OCH₃, —CO₂CH₃, and —CH₃.

73. The compound of embodiment 72, wherein n is 1.
74. The compound of embodiment 72, wherein n is 0.
75. The compound of any one of embodiments 72-74, wherein X is NR⁴.
76. The compound of any one of embodiments 72-75, wherein Y is CR⁴R′.
77. The compound of any one of embodiments 72-76, wherein R¹ is a heteroaryl selected from the group consisting of pyrimidinyi, pyridinyi, pyridazine, and pyrazine.
78. The compound of any one of embodiments 72-77, wherein R¹ is pyridinyl.
79. The compound of any one of embodiments 72-76, wherein R¹ is phenyl.
80. The compound of any one of embodiments 72-79, wherein R² and R³ are independently selected from the group consisting of H, F, C₁₋₆ alkyl, C₃₋₆ cycloalkyl, —(CH₂)—C₃₋₆ cycloalkyl, 4- to 6-membered heterocyclyl, and —(CH₂) (4- to 6-membered heterocyclyl).
81. The compound of any one of embodiments 72-79, wherein R² and R³ taken together with the carbon atom to which they are attached form a C₃₋₆ cycloalkyl.
82. The compound of any one of embodiments 72-79 wherein R² and R³ taken together with the carbon atom to which they are attached form a cyclopropyl.
83. The compound of any one of embodiments 72-79, wherein R² and R³ taken together with the carbon atom to which they are attached form a 4- to 6-membered heterocyclyl.
84. The compound of any one of embodiments 72-80, wherein R² is H and R³ is C₃₋₆ cycloalkyl.
85. The compound of any one of embodiments 72-80, wherein R² is H and R³ is cyclopropyl.
86. The compound of any one of embodiments 72-80, wherein R² and R³ are C₁₋₆ alkyl.
87. The compound of any one of embodiments 72-80, wherein R² and R³ are methyl.
88. The compound of any one of embodiments 72-87, wherein R⁴ is selected from the group consisting of H, alkyl; carbocyclyl, heterocyclyl, —(CH₂)-carbocyclyl, and —(CH₂)-heterocyclyl.
89. The compound of any one of embodiments 72-88, wherein R⁴ is H.
90. The compound of any one of embodiments 72-88, wherein R⁴ is —(CH₂)-heterocyclyl.
91. The compound of any one of embodiments 72-88, wherein R⁴ is —(CH₂)-oxetane.
92. The compound of any one of embodiments 72-88, wherein R⁴ is alkyl.
93. The compound of any one of embodiments 72-88, wherein R⁴ is methyl.
94. The compound of embodiment 1, wherein the compound is a compound of Formula (IIa):

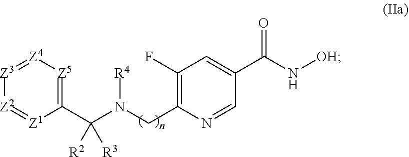

wherein
Z¹, Z², Z³, Z⁴ and Z⁵ are independently selected from N and CR⁵;
wherein R⁵ is independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-heteroaryl, —SO₂-alkyl, and —CN.
95. The compound of embodiment 94, wherein Z¹, Z², Z³, Z⁴ and Z⁵ are CR⁵.
96. The compound of embodiment 94, wherein Z¹ is N and Z², Z³, Z⁴ and Z⁵ are CR⁵.
97. The compound of embodiment 94, wherein Z² is N and Z¹, Z³, Z⁴ and Z⁵ are CR⁵.
98. The compound of embodiment 94, wherein Z³ is N and Z¹, Z², Z⁴ and Z⁵ are CR⁵.
99. The compound of any one of embodiments 94-98, wherein n is 1.
100. The compound of any one of embodiments 94-98, wherein n is 0.
101. The compound of any one of embodiments 94-100, wherein R² and R³ are independently selected from the group consisting of H, F, C₁₋₆ alkyl, C₃₋₆ cycloalkyl, cycloalkyl, 4- to 6-membered heterocyclyl, and —(CH₂)-(4- to 6-membered heterocyclyl).
102. The compound of any one of embodiments 94-100, wherein R² and R³ taken together with the carbon atom to which they are attached form a C₃₋₆ cycloalkyl.
103. The compound of any one of embodiments 94-100, wherein R² and R³ taken together with the carbon atom to which they are attached form a cyclopropyl.
104. The compound of any one of embodiments 94-100, wherein R² and R³ taken together with the carbon atom to which they are attached form a 4- to 6-membered heterocyclyl.
105. The compound of any one of embodiments 94-100, wherein R² is H and R³ is C₃₋₆ cycloalkyl.
106. The compound of any one of embodiments 94-100, wherein R² is and R³ is cyclopropyl.
107. The compound of any one of embodiments 94-100, wherein R² and R³ are C₁₋₆ alkyl.
108. The compound of any one of embodiments 94-100, wherein R² and R³ are methyl.
109. The compound of any one of embodiments 94-108, wherein R⁴ is selected from the group consisting of H, alkyl, carbocyclyl, heterocyclyl, —(CH₂)-carbocyclyl, and —(CH₂)-heterocyclyl.
110. The compound of any one of embodiments 94-109, wherein R⁴ is H.
111. The compound of any one of embodiments 94-109, wherein R⁴ is —(CH₂)-heterocyclyl.
112. The compound of any one of embodiments 94-109, wherein R⁴ is —(CH₂)-oxetane.
113. The compound of any one of embodiments 94-109, wherein R⁴ is alkyl.
114. The compound of any one of embodiments 94-109, wherein R⁴ is methyl.
115. The compound of any one of embodiments 94-114, wherein R⁵ is independently selected from H and halogen.
116. The compound of any one of embodiments 94-114, wherein R⁵ is independently selected from H and fluoro.
117. The compound of any one of embodiments 94 and 99-114, wherein the compound is a compound of Formula (IIb):

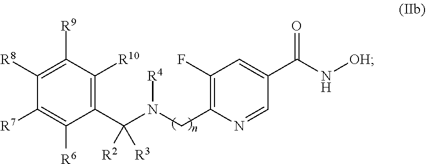

wherein
R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.
118. The compound of embodiment 117, wherein R⁶, R⁷, R⁸, R⁹, and R¹⁰ are independently selected from the group consisting of H and halogen.
119. The compound of embodiment 117, wherein R⁶ and R¹⁰ are halogen and R⁷, R⁸, R⁹, and R¹⁰ are H.
120. The compound of embodiment 117, wherein R⁶ and R¹⁰ are fluoro and R⁷, R⁸, and R⁹ are H.
121. The compound of any one of embodiments 94 and 99-114, wherein the compound is a compound of Formula (IIc):

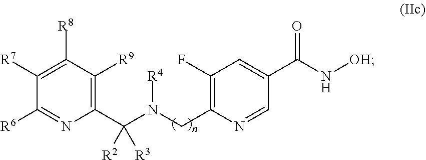

wherein
R⁶, R⁷, R⁸, and R⁹ are independently selected from the group consisting of H, halogen, alkyl, haloalkyl, carbocyclyl, heterocyclyl, aryl, heteroary, —CO₂H, —CO₂-alkyl, —O-alkyl, —O-haloalkyl, —O-aryl, —O-heteroaryl, —SO₂-alkyl, and —CN.
122. The compound of embodiment 72, wherein the compound is selected from the group consisting of:

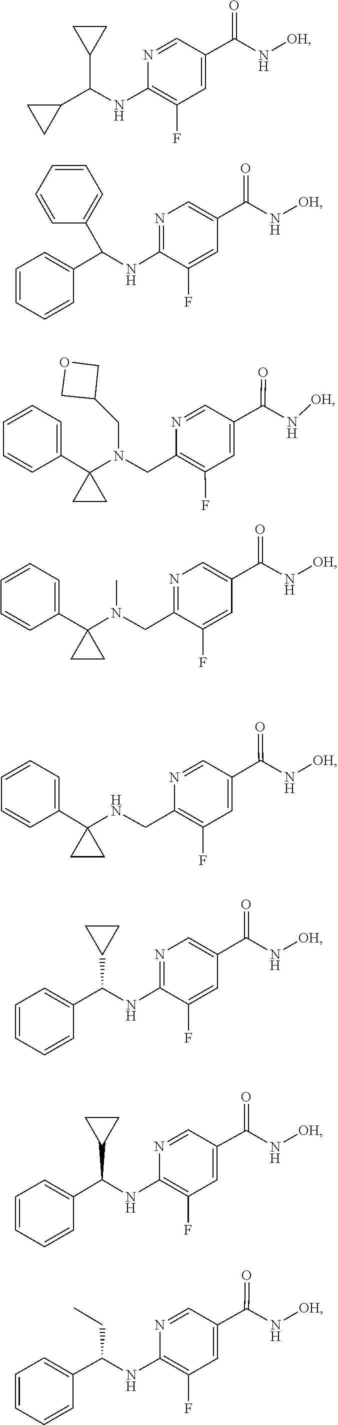

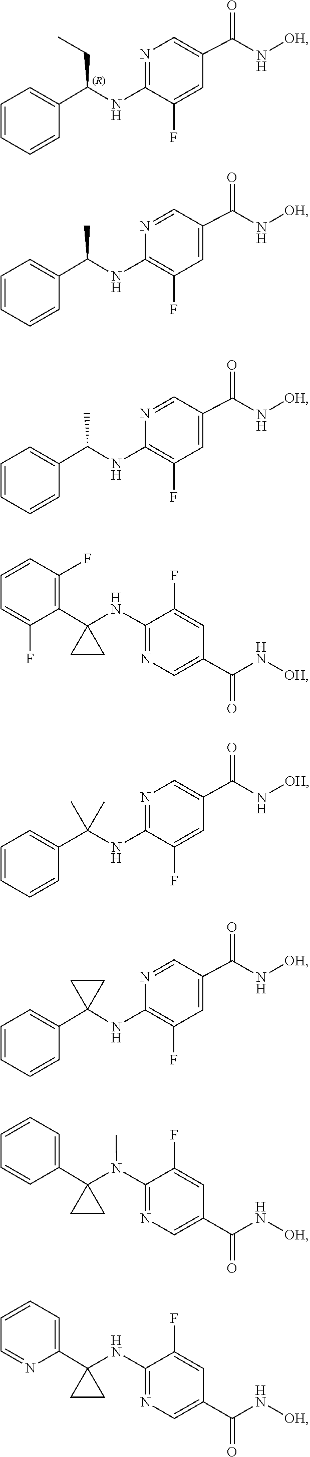

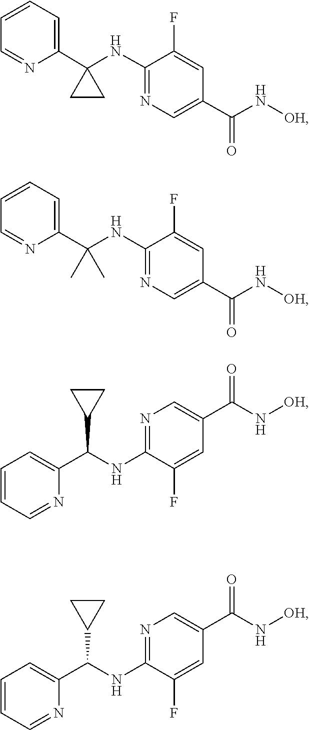

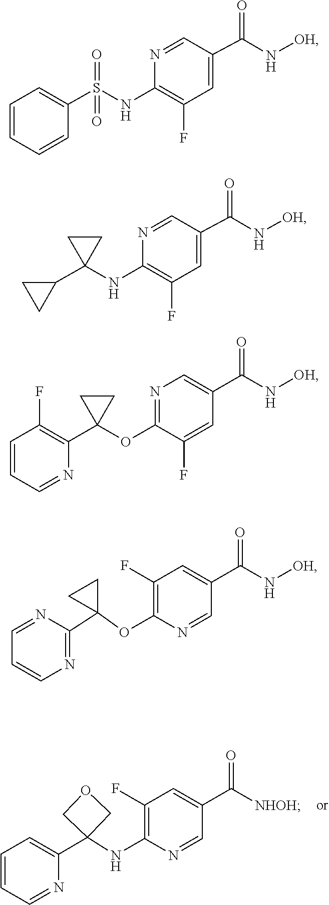

a pharmaceutically acceptable salt thereof.
123. The compound of embodiment 1, which is a compound of Formula (III), or pharmaceutically acceptable salt thereof:

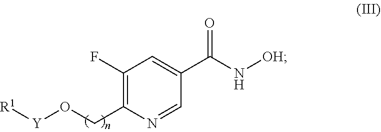

wherein

n is 0 or 1;

Y is a bond or CR³R³;

R¹ is selected from the group consisting of carbocyclyl, heterocyclyl, aryl, and heteroaryl;

R² and R³ are independently selected from the group consisting of H, halogen, alkyl, carbocyclyl, heterocyclyl, aryl, heteroaryl, —(CH₂)-carbocyclyl, —(CH₂)-heterocyclyl, (CH₂)-aryl, and —(CH₂)-heteroaryl; or

R¹ and R² when present taken together with the carbon atom to which they are attached form a carbocyclyl or heterocyclyl; or

R² and R³ taken together with the carbon atom to which they are attached form a carbocyclyl or heterocyclyl; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

124. The compound of embodiment 123, wherein Y is a bond.
125. The compound of embodiment 123, wherein Y is CR²CR³.
126. The compound of any one of embodiments 123-125, wherein n is 1.
127. The compound of any one of embodiments 123-125, wherein n is 0.
128. The compound of any one of embodiments 123-127, wherein R¹ is selected from the group consisting of carbocyclyl, heterocyclyl, aryl, and heteroaryl.
129. The compound of any one of embodiments 123-128, wherein R¹ is a heteroaryl selected from the group consisting of pyrimidinyl, pyridinyl, pyridazine, and pyrazine.
130. The compound of any one of embodiments 123-128, wherein R¹ is pyridinyl.
131. The compound of any one of embodiments 123-128, wherein R¹ is phenyl.
132. The compound of any one of embodiments 123-127, wherein Y is a bond, and R¹ is H.
133. The compound of any one of embodiments 123-124 or 126-127, wherein R¹ and R² taken together with the carbon atom to which they are attached form a C₃₋₁₂ carbocyclyl.
134. The compound of any one of embodiments 123-132, wherein R² and R³ are independently selected from the group consisting of H, C₁₋₆ alkyl, C₃₋₆ cycloalkyl, —(CH₂) C₃₋₆ cycloalkyl, 4- to 6-membered heterocyclyl, and —(CH₂)—(4- to 6-membered heterocyclyl).
135. The compound of any one of embodiments 123-132, wherein R² and R³ taken together with the carbon atom to which they are attached form a C₃₋₆ cycloalkyl.
136. The compound of any one of embodiments 123-132, wherein R² and R³ taken together with the carbon atom to which they are attached form a cyclopropyl.
137. The compound of any one of embodiments 123-132, wherein R² and R³ taken together with the carbon atom to which they are attached form a 4- to 6-membered heterocyclyl.
138. The compound of any one of embodiments 123-132, wherein R² is H and R³ is C₃₋₆ cycloalkyl.
139. The compound of any one of embodiments 123-132, wherein R² is H and R³ is cyclopropyl.
140. The compound of any one of embodiments 123-132, wherein R² and R³ are C₁₋₆ alkyl.
141. The compound of any one of embodiments 123-132, wherein R² and R³ are methyl.
142. The compound of embodiment 123, wherein the compound is selected from the group consisting of:

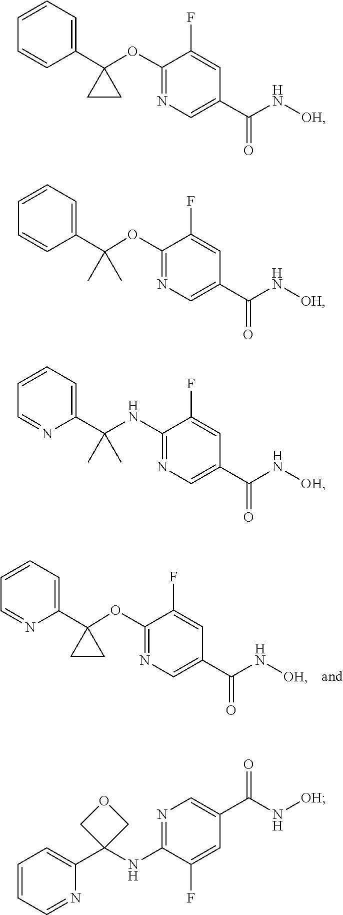 [SMILES: O=C(NO)c1cnc(OC2(c3ccccc3)CC2)c(F)c1 ; CC(C)(Oc1ncc(C(=O)NO)cc1F)c1ccccc1 ; CC(C)(Nc1ncc(C(=O)NO)cc1F)c1ccccn1 ; O=C(NO)c1cnc(OC2(c3ccccn3)CC2)c(F)c1 ; O=C(NO)c1cnc(NC2(c3ccccn3)COC2)c(F)c1]

or a pharmaceutically acceptable salt thereof.
143. A compound of Formula (IV), or pharmaceutically acceptable salt thereof:

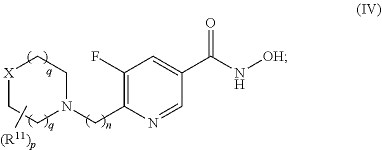

wherein:

n is 0 or 1;

p is 0, 1, or 4;

q is each independently 0, 1, or 2;

X is O, S(O)₂, NR¹², or CHR¹²;

R¹¹ is each independently H, F, alkyl, or oxo; or

two adjacent R¹¹ taken together with the carbon atoms to which they are attached form an aryl, heteroaryl, or heterocyclyl ring; or

two non-adjacent R¹¹ taken together with the atoms to which they are attached form a carbocyclyl or heterocyclyl ring;

R¹² is selected from the group consisting of alkyl, carbocyclyl, heterocyclyl, aryl, heteroaryl, —(CH₂)-carbocycyl, —(CH₂)-heterocyclyl, —(CH₂)-aryl, and —(CH₂)-heteroaryl; or

R¹¹ and R¹² taken together with the carbon and/or nitrogen atoms to which they are attached form an aryl, heteroaryl ring, or heterocyclyl ring; and

wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

144. The compound of embodiment 143, wherein n is 1.
145. The compound of embodiment 143, wherein n is 0.
146. The compound of any one of embodiments 143-145, wherein q is 1.
147. The compound of any one of embodiments 143-145, wherein q is 0.
148. The compound of any one of embodiments 143-146, wherein X is O.
149. The compound of any one of embodiments 143-146, wherein X is S(O)₂.
150. The compound of any one of embodiments 143-146, wherein X is NR¹².
151. The compound of any of embodiments 143-146, wherein X is CHR¹².
152. The compound of embodiment 150 or 151, wherein R¹² is H, Me, or Ph.
153. The compound of any one of embodiments 143-152, wherein p is 4.
154. The compound of any one of embodiments 143-152, wherein p is 3.
155. The compound of any one of embodiments 143-152, wherein p is 2.
156. The compound of any one of embodiments 143-152, wherein p is 1.
157. The compound of any one of embodiments 143-152, wherein p is 0.
158. The compound of any one of embodiments 143-156, R¹¹ is oxo.
159. The compound of any one of embodiments 143-155, wherein two adjacent R¹¹ taken together with the carbon atoms to which they are attached form an aryl ring.
160. The compound of embodiment 159, wherein the aryl ring is a phenyl ring.
161. The compound of any one of embodiments 143-155, wherein two adjacent R¹¹ taken together with the carbon atoms to which they are attached form a heteroaryl ring or a heterocyclyl ring.
162. The compound of embodiment 161, wherein the heteroaryl ring is a pyridinyl ring.
163. The compound of embodiment 161, wherein the heterocyclyl ring is:

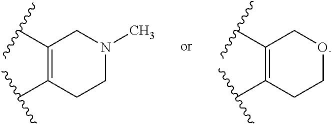

164. The compound of any one of embodiments 143-147 or 153-156, wherein when X is CHR¹², an R¹¹ and an R¹² taken together with the carbon atoms to which they are attached form an aryl ring.
165. The compound of embodiment 164, wherein the aryl ring is a phenyl ring.
166. The compound of any one of embodiments 143-147 or 153-156, wherein when X is NR¹², an R¹¹ and an R¹² taken together with the carbon and nitrogen atoms to which they are attached form a heteroaryl ring or a heterocyclyl ring.
167 The compound of embodiment 166, wherein the heteroaryl ring is a pyridinyl ring.
168. The compound of any one of embodiments 143-152, wherein when p is 4, two adjacent R¹¹ taken together with the carbon atoms to which they are attached form an aryl ring and two adjacent R¹¹ taken together with the carbon atoms to which they are attached form a heterocyclyl ring.
169. The compound of embodiments 168, the aryl ring is a phenyl ring.
170. The compound of embodiments 168 or 169, wherein the heterocyclyl ring is

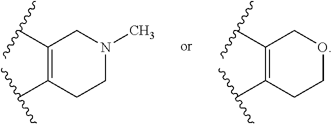

171. The compound of embodiment 143, wherein the compound of Formula (IV) is selected from the group consisting of:

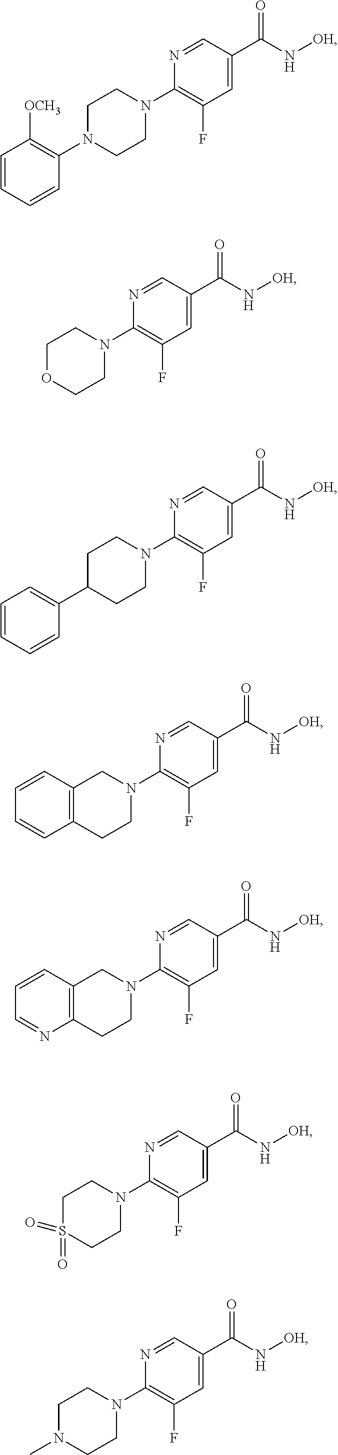

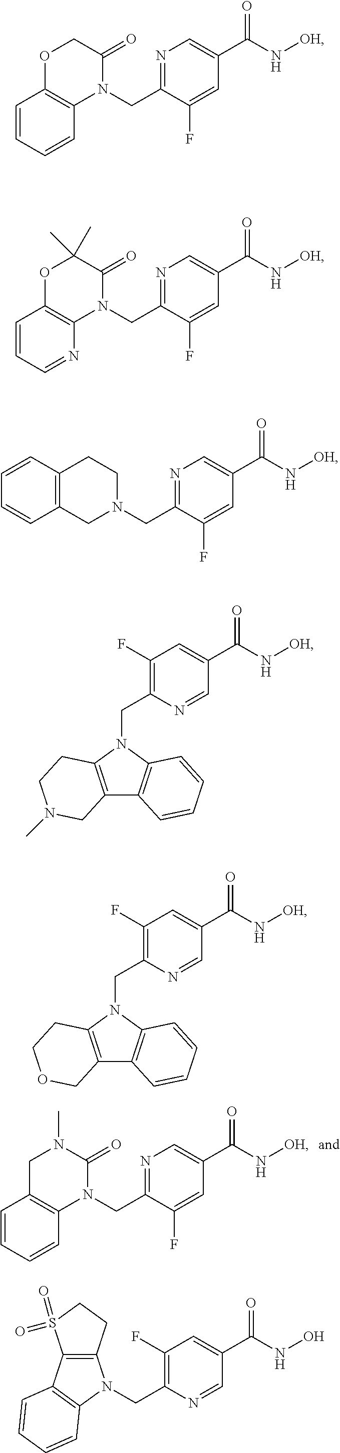

or a pharmaceutically acceptable salt thereof.
172. A composition comprising a compound of any one of embodiments 1-171 and a pharmaceutically acceptable excipient.
173. A method of improving sarcomere quality or preventing sarcomere damage in cardiomyocytes, comprising contacting a cardiomyocyte with an effective amount of the compound of any one of embodiments 1-171 or the composition of embodiment 172, wherein the method induces an improvement in sarcomere quality.
174. The method of embodiment 173, wherein the improvement in sarcomere quality is measured using an artificial intelligence algorithm.
175. The method of embodiment 174, wherein the algorithm is trained to build neuronal net models to segregate classes of cells based on cardiomyocytes with sarcomere damage against cardiomyocytes with limited sarcomere damage.
176. A method of increasing tubulin acetylation in cardiomyocytes with an effective amount of the compound of any one of embodiments 1-171 or the composition of embodiment 172, wherein the method increases levels of tubulin acetylation in cardiomyocytes.
177. The method of any one of embodiments 173-176, wherein the cardiomyocyte is an in vivo cardiomyocyte.
178. A method of treating heart disease in a subject in need thereof, comprising administering a therapeutically effective amount of the compound of any one of embodiments 1-171 or the composition of embodiment 172 to the subject.
179. The method of embodiment 178, wherein the heart disease is dilated cardiomyopathy (DCM), hypertrophic cardiomyopathy (HCM), restrictive cardiomyopathy (RCM), left ventricular non-compaction (LVNC), arrhythmogenic right ventricular cardiomyopathy (ARVC), or arrhythmogenic right ventricular dysplasia (ARVD).
180. The method of embodiment 178, wherein the heart disease is myocardial infarction.
181. The method of any one of embodiments 178-180, wherein the method causes at least one of the following effects in cardiomyocytes: increased tubulin acetylation, increased contractility, reduced sarcomere damage, increase in autophagy.

1. A compound of Formula (I), or pharmaceutically acceptable salt thereof: 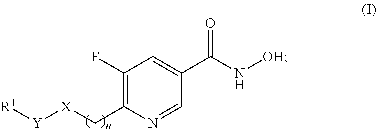
wherein
n is 0 or 1;
X is O, NR⁴, or CR⁴R⁴;
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R⁴′ are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R⁴′ taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

2. The compound of claim 1, wherein n is 1.

3. The compound of claim 1, wherein n is 0.

4. The compound of claim 1, wherein X is NR⁴ or CR⁴R^{4′.}

5. The compound of claim 1, wherein Y is CR²R³.

6. The compound of claim 1, wherein R¹ is a heteroaryl selected from the group consisting of pyrimidinyl, pyridinyl, pyridazine, and pyrazine.

7. The compound of claim 1, wherein R¹ is phenyl.

8. The compound of claim 1, wherein R² and R³ are independently selected from the group consisting of H, F, C₁₋₆ alkyl, C₃₋₆ cycloalkyl, —(CH₂)—C₃₋₆ cycloalkyl, 4- to 6-membered heterocyclyl, and —(CH₂)-(4- to 6-membered heterocyclyl).

9. The compound of claim 1, wherein R² and R³ taken together with the carbon atom to which they are attached form a cyclopropyl.

10. The compound of claim 1, wherein R⁴ is selected from the group consisting of H, alkyl, carbocyclyl, heterocyclyl, —(CH₂)-carbocyclyl, and —(CH₂)-heterocyclyl.

11. The compound of claim 1, wherein R⁴ and R⁴′ are each H.

12. The compound of claim 1, wherein the compound is selected from the group consisting of: 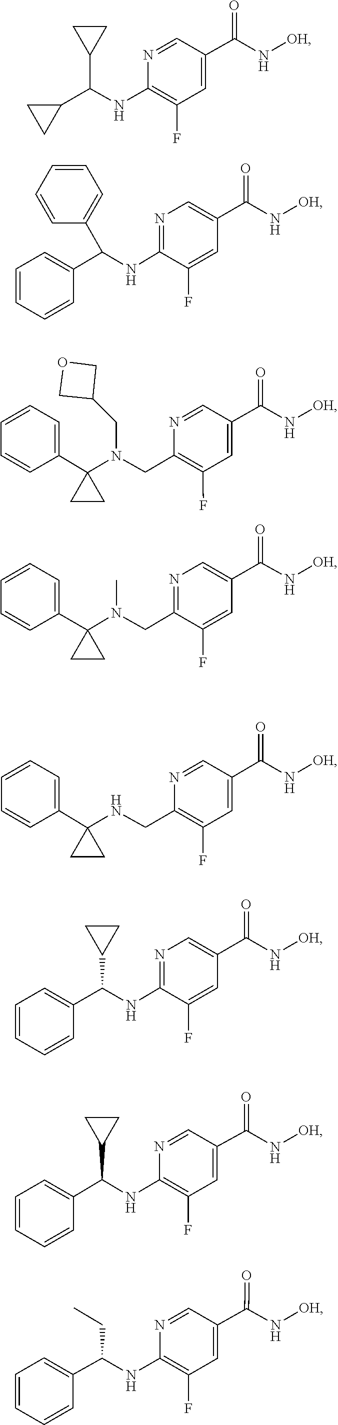 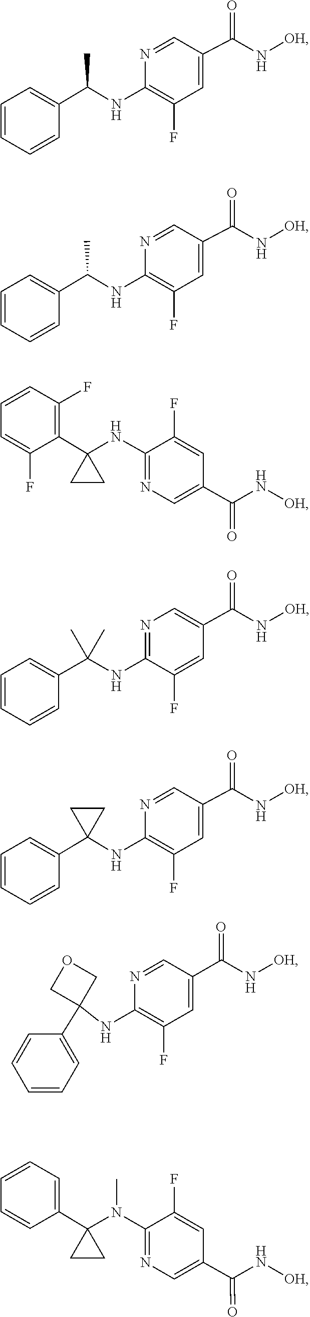 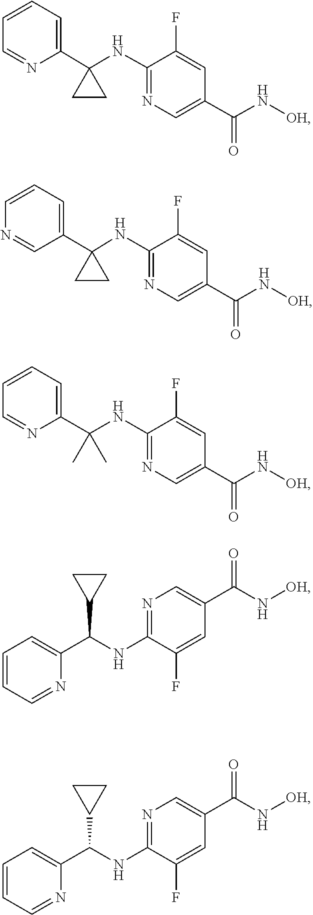 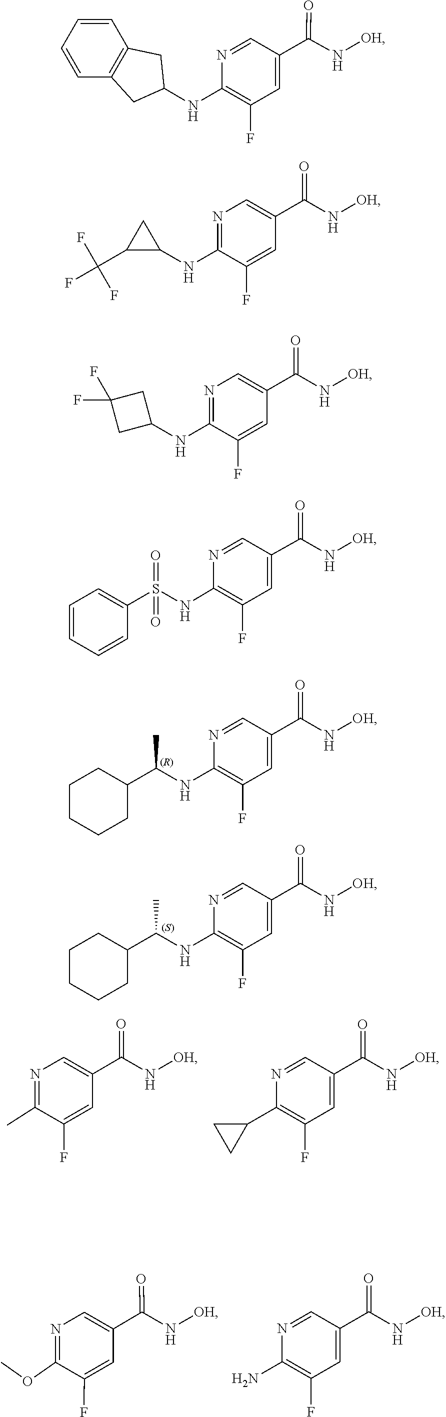 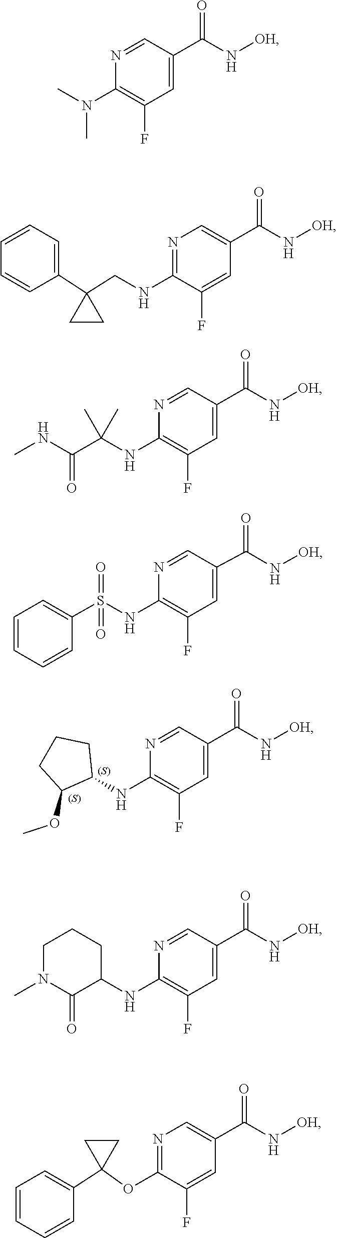 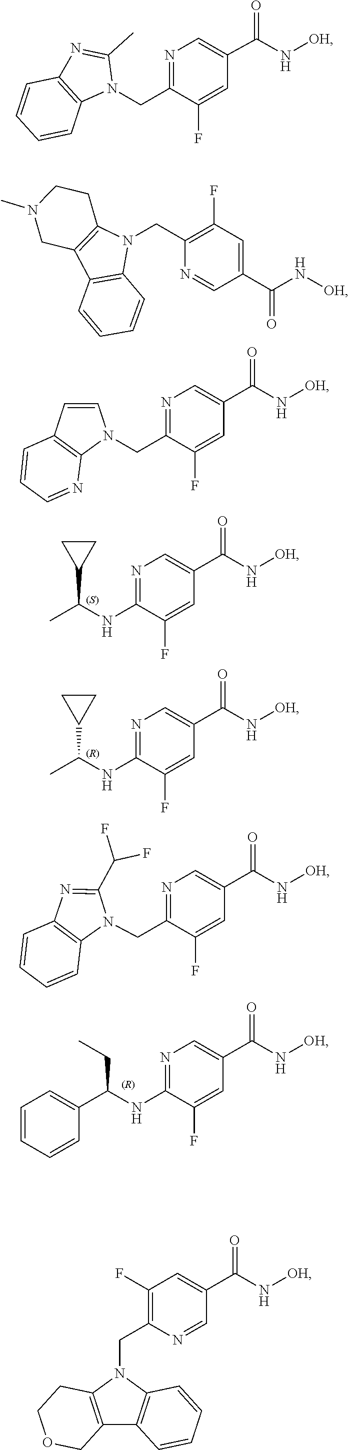 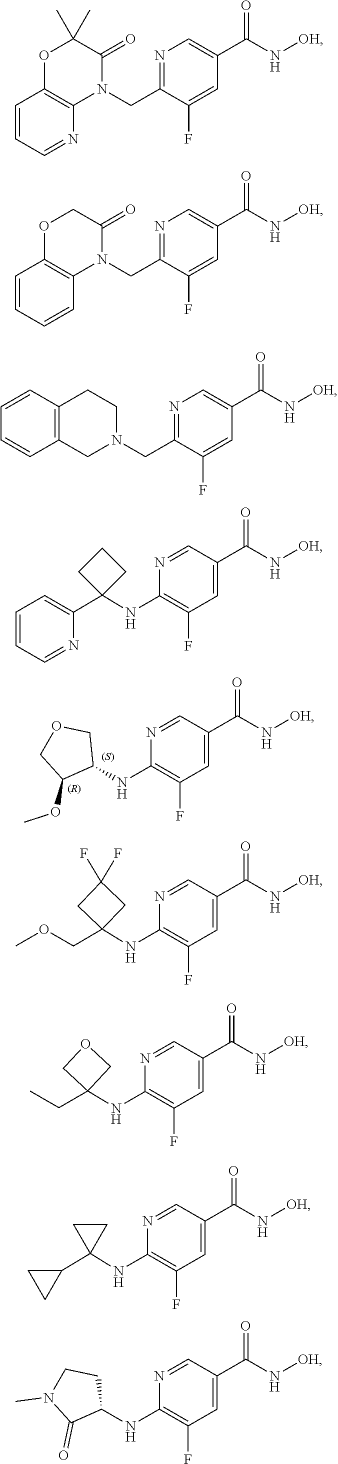 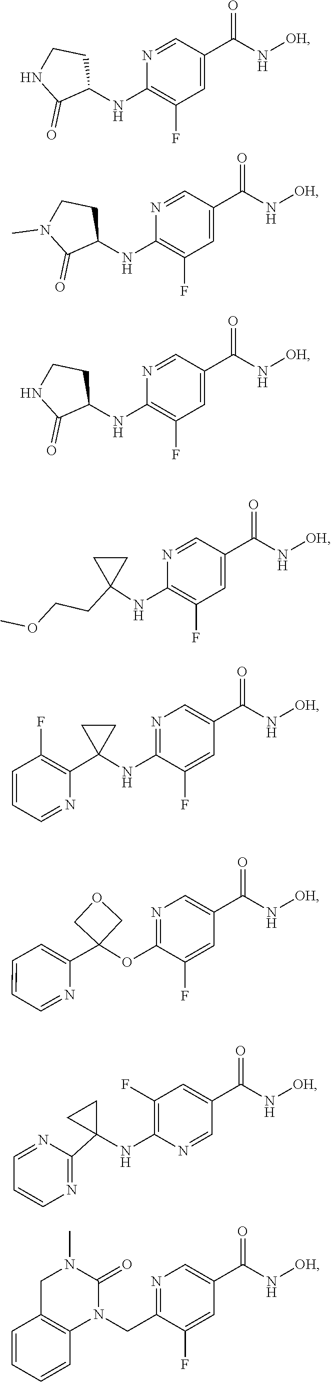 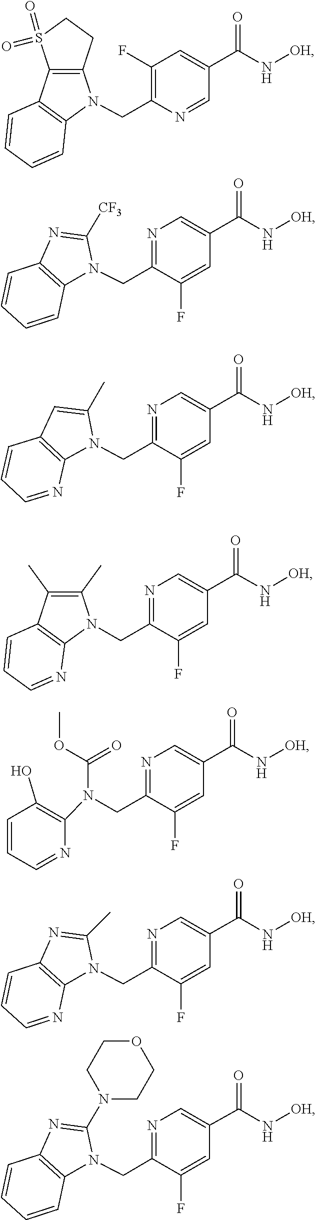 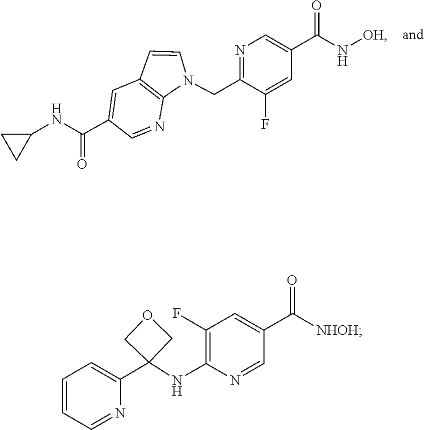
or a pharmaceutically acceptable salt thereof.

13. The compound of claim 1, wherein
n is 0 or 1;
X is NR⁴ or CR⁴R⁴;
Y is CR²R³ or S(O)₂;
R is selected from the group consisting of carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl, or
R² and R³ taken together with the carbon atom to which they are attached form a carbocyclyl or heterocyclyl; and
R⁴ and R⁴′ are independently selected from the group consisting of H, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R⁴′ taken together with the carbon atom to which they are attached form a carbocyclyl or heterocyclyl; and
wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

14. The compound of claim 13, wherein Y is CR⁴R′.

15. The compound of claim 1, wherein the compound is a compound of Formula (IIa): 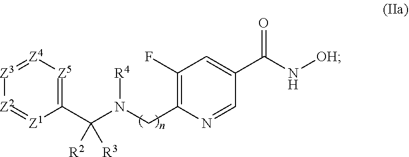
wherein
Z¹, Z², Z³, Z⁴ and Z⁵ are independently selected from N and CR⁵;
wherein R⁵ is independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.

16. The compound of claim 15, wherein the compound is a compound of Formula (IIb): 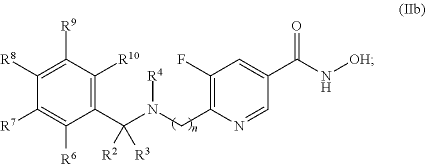
wherein
R⁶, R⁷, R⁸, R⁹, and R¹⁰ are independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.

17. The compound of claim 16, wherein R⁶, R⁷, R⁸, R⁹, and R₁₀ are independently selected from the group consisting of H and halogen.

18. The compound of claim 15, wherein the compound is a compound of Formula (IIc): 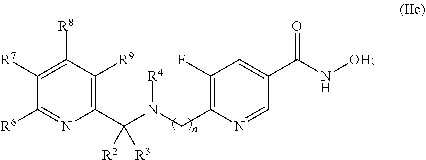
wherein
R⁶, R⁷, R⁸, and R⁹ are independently selected from the group consisting of H, halogen, alkyl, haloalkyl, carbocyclyl, heterocyclyl, aryl, heteroaryl, —CO₂H, —CO₂-alkyl, —O-alkyl, —O-haloalkyl, —O-aryl, —O-heteroaryl, —SO₂-alkyl, and —CN.

19. The compound of claim 1, wherein the compound is selected from the group consisting of: 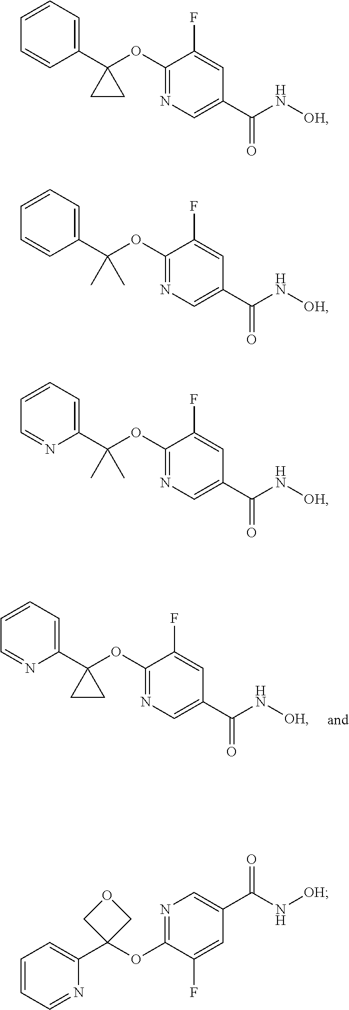 [SMILES: O=C(NO)c1cnc(OC2(c3ccccc3)CC2)c(F)c1 ; CC(C)(Oc1ncc(C(=O)NO)cc1F)c1ccccc1 ; CC(C)(Oc1ncc(C(=O)NO)cc1F)c1ccccn1 ; O=C(NO)c1cnc(OC2(c3ccccn3)CC2)c(F)c1 ; O=C(NO)c1cnc(OC2(c3ccccn3)COC2)c(F)c1]
or a pharmaceutically acceptable salt thereof.

20. A compound of Formula (IV), or pharmaceutically acceptable salt thereof: 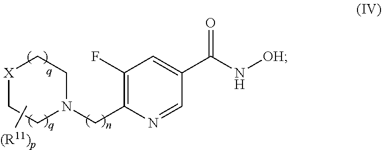
wherein:
n is 0 or 1;
p is 0, 1, 2, 3, or 4;
q is each independently 0, 1, or 2;
X is O, S(O)₂, NR¹², or CHR¹²;
R¹¹ is each independently H, F, alkyl, or oxo; or
two adjacent R¹¹ taken together with the carbon atoms to which they are attached form an aryl, heteroaryl, or heterocyclyl ring; or
two non-adjacent R¹¹ taken together with the atoms to which they are attached form a carbocyclyl or heterocyclyl ring;
R¹² is selected from the group consisting of alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹¹ and R¹² taken together with the carbon and/or nitrogen atoms to which they are attached form an aryl, heteroaryl ring, or heterocyclyl ring; and
wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

21. The compound of claim 20, wherein q is 1.

22. The compound of claim 20, wherein two adjacent taken together with the carbon atoms to which they are attached form an aryl ring.

23. The compound of claim 20, wherein the compound of Formula (IV) is selected from the group consisting of: 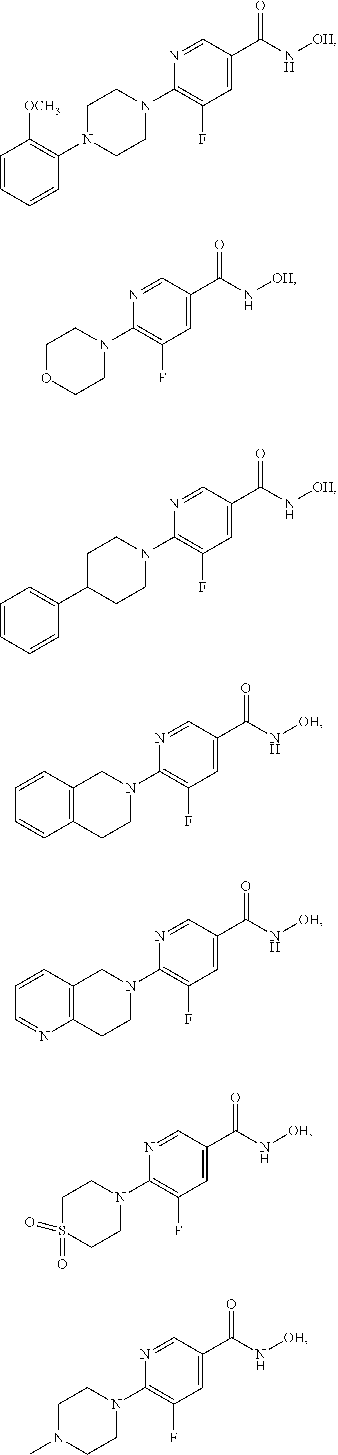 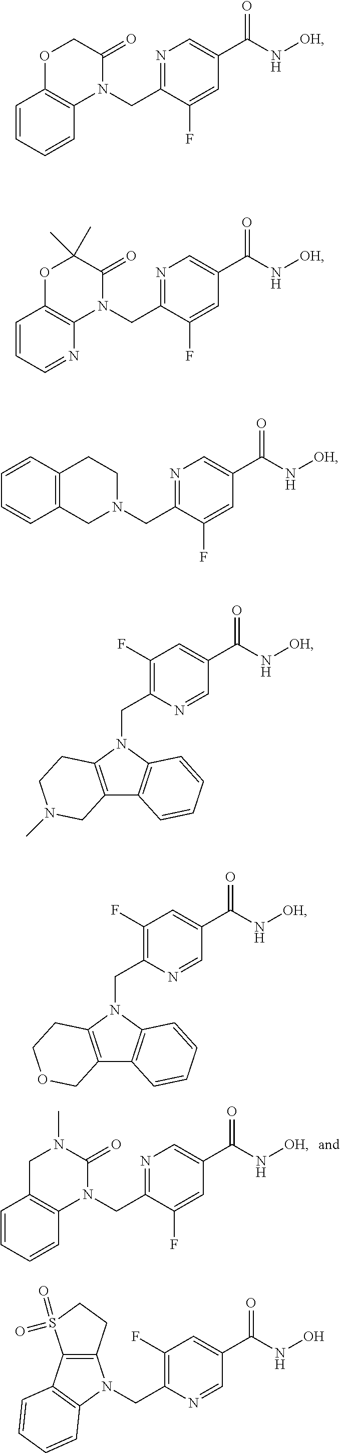
or a pharmaceutically acceptable salt thereof.

24. A composition comprising a compound of claim 1 and a pharmaceutically acceptable excipient.

25. A method of improving sarcomere quality or preventing sarcomere damage in cardiomyocytes, comprising contacting a cardiomyocyte with the composition of claim 24, wherein the method induces an improvement in sarcomere quality.

26. A method of increasing tubulin acetylation in cardiomyocytes with the composition of claim 24, wherein the method increases levels of tubulin acetylation in cardiomyocytes.

27. A method of treating heart disease in a subject in need thereof, comprising administering the composition of claim 24 to the subject.
